SEQUENCE LISTING (347 sequences)

SEQ ID NO: 1 - protein, 28 aa, synthetic construct
  source 1..28: note = Description of Artificial Sequence: Synthetic peptide
EVRLQQSGPDLIKPGASVKMSCKASGYT

SEQ ID NO: 2 - protein, 7 aa, synthetic construct
  source 1..7: note = Description of Artificial Sequence: Synthetic peptide
FTGYVMH

SEQ ID NO: 3 - protein, 14 aa, synthetic construct
  source 1..14: note = Description of Artificial Sequence: Synthetic peptide
WVKQRPGQGLEWIG

SEQ ID NO: 4 - protein, 17 aa, synthetic construct
  source 1..17: note = Description of Artificial Sequence: Synthetic peptide
FINPYNDDIQSNERFRG

SEQ ID NO: 5 - protein, 32 aa, synthetic construct
  source 1..32: note = Description of Artificial Sequence: Synthetic polypeptide
KATLTSDKSSTTAYMELSSLTSEDSAVYYCAR

SEQ ID NO: 6 - protein, 15 aa, synthetic construct
  source 1..15: note = Description of Artificial Sequence: Synthetic peptide
GAGYNFDGAYRFFDF

SEQ ID NO: 7 - protein, 11 aa, synthetic construct
  source 1..11: note = Description of Artificial Sequence: Synthetic peptide
WGQGTTLTVSS

SEQ ID NO: 8 - protein, 28 aa, synthetic construct
  source 1..28: note = Description of Artificial Sequence: Synthetic peptide
QVQLVQSGAEVKKPGASVKVSCKASGYT

SEQ ID NO: 9 - protein, 7 aa, synthetic construct
  source 1..7: note = Description of Artificial Sequence: Synthetic peptide
FTGYVMH

SEQ ID NO: 10 - protein, 14 aa, synthetic construct
  source 1..14: note = Description of Artificial Sequence: Synthetic peptide
WVRQAPGQGLEWMG

SEQ ID NO: 11 - protein, 17 aa, synthetic construct
  source 1..17: note = Description of Artificial Sequence: Synthetic peptide
FINPYNDDIQSNERFRG

SEQ ID NO: 12 - protein, 32 aa, synthetic construct
  source 1..32: note = Description of Artificial Sequence: Synthetic polypeptide
RVTMTSDKSTTTAYMELSSLRSEDTAVYYCAR

SEQ ID NO: 13 - protein, 15 aa, synthetic construct
  source 1..15: note = Description of Artificial Sequence: Synthetic peptide
GAGYNFDGAYRFFDF

SEQ ID NO: 14 - protein, 11 aa, synthetic construct
  source 1..11: note = Description of Artificial Sequence: Synthetic peptide
WGQGTLVTVSS

SEQ ID NO: 15 - protein, 28 aa, synthetic construct
  source 1..28: note = Description of Artificial Sequence: Synthetic peptide
QVQLVQSGAEVKKPGASVKVSCKASGYT

SEQ ID NO: 16 - protein, 7 aa, synthetic construct
  source 1..7: note = Description of Artificial Sequence: Synthetic peptide
FTGYVMH

SEQ ID NO: 17 - protein, 14 aa, synthetic construct
  source 1..14: note = Description of Artificial Sequence: Synthetic peptide
WVRQAPGQGLEWMG

SEQ ID NO: 18 - protein, 17 aa, synthetic construct
  source 1..17: note = Description of Artificial Sequence: Synthetic peptide
FINPYNDDIQSNERFRG

SEQ ID NO: 19 - protein, 32 aa, synthetic construct
  source 1..32: note = Description of Artificial Sequence: Synthetic polypeptide
WVTMTSDKSITTAYMELSRLRSDDTAVYYCAR

SEQ ID NO: 20 - protein, 15 aa, synthetic construct
  source 1..15: note = Description of Artificial Sequence: Synthetic peptide
GAGYNFDGAYRFFDF

SEQ ID NO: 21 - protein, 11 aa, synthetic construct
  source 1..11: note = Description of Artificial Sequence: Synthetic peptide
WGQGTLVTVSS

SEQ ID NO: 22 - protein, 28 aa, synthetic construct
  source 1..28: note = Description of Artificial Sequence: Synthetic peptide
QVQLVQSGAEVKKPGSSVKVSCKASGYT

SEQ ID NO: 23 - protein, 7 aa, synthetic construct
  source 1..7: note = Description of Artificial Sequence: Synthetic peptide
FTGYVMH

SEQ ID NO: 24 - protein, 14 aa, synthetic construct
  source 1..14: note = Description of Artificial Sequence: Synthetic peptide
WVRQAPGQGLEWMG

SEQ ID NO: 25 - protein, 17 aa, synthetic construct
  source 1..17: note = Description of Artificial Sequence: Synthetic peptide
FINPYNDDIQSNERFRG

SEQ ID NO: 26 - protein, 32 aa, synthetic construct
  source 1..32: note = Description of Artificial Sequence: Synthetic polypeptide
RVTITSDKSTTTAYMELSSLRSEDTAVYYCAR

SEQ ID NO: 27 - protein, 15 aa, synthetic construct
  source 1..15: note = Description of Artificial Sequence: Synthetic peptide
GAGYNFDGAYRFFDF

SEQ ID NO: 28 - protein, 11 aa, synthetic construct
  source 1..11: note = Description of Artificial Sequence: Synthetic peptide
WGQGTLVTVSS

SEQ ID NO: 29 - protein, 28 aa, synthetic construct
  source 1..28: note = Description of Artificial Sequence: Synthetic peptide
QVQLVQSGAEVKKPGASVKVSCKASGYT

SEQ ID NO: 30 - protein, 7 aa, synthetic construct
  source 1..7: note = Description of Artificial Sequence: Synthetic peptide
FTGYVMH

SEQ ID NO: 31 - protein, 14 aa, synthetic construct
  source 1..14: note = Description of Artificial Sequence: Synthetic peptide
WVRQAPGQRLEWMG

SEQ ID NO: 32 - protein, 17 aa, synthetic construct
  source 1..17: note = Description of Artificial Sequence: Synthetic peptide
FINPYNDDIQSNERFRG

SEQ ID NO: 33 - protein, 32 aa, synthetic construct
  source 1..32: note = Description of Artificial Sequence: Synthetic polypeptide
RVTITSDKSATTAYMELSSLRSEDTAVYYCAR

SEQ ID NO: 34 - protein, 15 aa, synthetic construct
  source 1..15: note = Description of Artificial Sequence: Synthetic peptide
GAGYNFDGAYRFFDF

SEQ ID NO: 35 - protein, 11 aa, synthetic construct
  source 1..11: note = Description of Artificial Sequence: Synthetic peptide
WGQGTLVTVSS

SEQ ID NO: 36 - protein, 30 aa, synthetic construct
  source 1..30: note = Description of Artificial Sequence: Synthetic polypeptide
EVRLQQSGPDLIKPGASVKMSCKASGYTFT

SEQ ID NO: 37 - protein, 5 aa, synthetic construct
  source 1..5: note = Description of Artificial Sequence: Synthetic peptide
GYVMH

SEQ ID NO: 38 - protein, 14 aa, synthetic construct
  source 1..14: note = Description of Artificial Sequence: Synthetic peptide
WVKQRPGQGLEWIG

SEQ ID NO: 39 - protein, 17 aa, synthetic construct
  source 1..17: note = Description of Artificial Sequence: Synthetic peptide
FINPYNDDIQSNERFRG

SEQ ID NO: 40 - protein, 32 aa, synthetic construct; identical to SEQ ID NO: 5
  source 1..32: note = Description of Artificial Sequence: Synthetic polypeptide

SEQ ID NO: 41 - protein, 15 aa, synthetic construct
  source 1..15: note = Description of Artificial Sequence: Synthetic peptide
GAGYNFDGAYRFFDF

SEQ ID NO: 42 - protein, 11 aa, synthetic construct
  source 1..11: note = Description of Artificial Sequence: Synthetic peptide
WGQGTTLTVSS

SEQ ID NO: 43 - protein, 30 aa, synthetic construct
  source 1..30: note = Description of Artificial Sequence: Synthetic polypeptide
QVQLVQSGAEVKKPGASVKVSCKASGYTFT

SEQ ID NO: 44 - protein, 5 aa, synthetic construct
  source 1..5: note = Description of Artificial Sequence: Synthetic peptide
GYVMH

SEQ ID NO: 45 - protein, 14 aa, synthetic construct
  source 1..14: note = Description of Artificial Sequence: Synthetic peptide
WVRQAPGQGLEWMG

SEQ ID NO: 46 - protein, 17 aa, synthetic construct
  source 1..17: note = Description of Artificial Sequence: Synthetic peptide
FINPYNDDIQSNERFRG

SEQ ID NO: 47 - protein, 32 aa, synthetic construct; identical to SEQ ID NO: 12
  source 1..32: note = Description of Artificial Sequence: Synthetic polypeptide

SEQ ID NO: 48 - protein, 15 aa, synthetic construct
  source 1..15: note = Description of Artificial Sequence: Synthetic peptide
GAGYNFDGAYRFFDF

SEQ ID NO: 49 - protein, 11 aa, synthetic construct
  source 1..11: note = Description of Artificial Sequence: Synthetic peptide
WGQGTLVTVSS

SEQ ID NO: 50 - protein, 30 aa, synthetic construct; identical to SEQ ID NO: 43
  source 1..30: note = Description of Artificial Sequence: Synthetic polypeptide

SEQ ID NO: 51 - protein, 5 aa, synthetic construct
  source 1..5: note = Description of Artificial Sequence: Synthetic peptide
GYVMH

SEQ ID NO: 52 - protein, 14 aa, synthetic construct
  source 1..14: note = Description of Artificial Sequence: Synthetic peptide
WVRQAPGQGLEWMG

SEQ ID NO: 53 - protein, 17 aa, synthetic construct
  source 1..17: note = Description of Artificial Sequence: Synthetic peptide
FINPYNDDIQSNERFRG

SEQ ID NO: 54 - protein, 32 aa, synthetic construct; identical to SEQ ID NO: 19
  source 1..32: note = Description of Artificial Sequence: Synthetic polypeptide

SEQ ID NO: 55 - protein, 15 aa, synthetic construct
  source 1..15: note = Description of Artificial Sequence: Synthetic peptide
GAGYNFDGAYRFFDF

SEQ ID NO: 56 - protein, 11 aa, synthetic construct
  source 1..11: note = Description of Artificial Sequence: Synthetic peptide
WGQGTLVTVSS

SEQ ID NO: 57 - protein, 30 aa, synthetic construct
  source 1..30: note = Description of Artificial Sequence: Synthetic polypeptide
QVQLVQSGAEVKKPGSSVKVSCKASGYTFT

SEQ ID NO: 58 - protein, 5 aa, synthetic construct
  source 1..5: note = Description of Artificial Sequence: Synthetic peptide
GYVMH

SEQ ID NO: 59 - protein, 14 aa, synthetic construct
  source 1..14: note = Description of Artificial Sequence: Synthetic peptide
WVRQAPGQGLEWMG

SEQ ID NO: 60 - protein, 17 aa, synthetic construct
  source 1..17: note = Description of Artificial Sequence: Synthetic peptide
FINPYNDDIQSNERFRG

SEQ ID NO: 61 - protein, 32 aa, synthetic construct; identical to SEQ ID NO: 26
  source 1..32: note = Description of Artificial Sequence: Synthetic polypeptide

SEQ ID NO: 62 - protein, 15 aa, synthetic construct
  source 1..15: note = Description of Artificial Sequence: Synthetic peptide
GAGYNFDGAYRFFDF

SEQ ID NO: 63 - protein, 11 aa, synthetic construct
  source 1..11: note = Description of Artificial Sequence: Synthetic peptide
WGQGTLVTVSS

SEQ ID NO: 64 - protein, 30 aa, synthetic construct; identical to SEQ ID NO: 43
  source 1..30: note = Description of Artificial Sequence: Synthetic polypeptide

SEQ ID NO: 65 - protein, 5 aa, synthetic construct
  source 1..5: note = Description of Artificial Sequence: Synthetic peptide
GYVMH

SEQ ID NO: 66 - protein, 14 aa, synthetic construct
  source 1..14: note = Description of Artificial Sequence: Synthetic peptide
WVRQAPGQRLEWMG

SEQ ID NO: 67 - protein, 17 aa, synthetic construct
  source 1..17: note = Description of Artificial Sequence: Synthetic peptide
FINPYNDDIQSNERFRG

SEQ ID NO: 68 - protein, 32 aa, synthetic construct; identical to SEQ ID NO: 33
  source 1..32: note = Description of Artificial Sequence: Synthetic polypeptide

SEQ ID NO: 69 - protein, 15 aa, synthetic construct
  source 1..15: note = Description of Artificial Sequence: Synthetic peptide
GAGYNFDGAYRFFDF

SEQ ID NO: 70 - protein, 11 aa, synthetic construct
  source 1..11: note = Description of Artificial Sequence: Synthetic peptide
WGQGTLVTVSS

SEQ ID NO: 71 - protein, 30 aa, synthetic construct; identical to SEQ ID NO: 57
  source 1..30: note = Description of Artificial Sequence: Synthetic polypeptide

SEQ ID NO: 72 - protein, 5 aa, synthetic construct
  source 1..5: note = Description of Artificial Sequence: Synthetic peptide
GYVMH

SEQ ID NO: 73 - protein, 14 aa, synthetic construct
  source 1..14: note = Description of Artificial Sequence: Synthetic peptide
WVRQAPGQGLEWMG

SEQ ID NO: 74 - protein, 17 aa, synthetic construct
  source 1..17: note = Description of Artificial Sequence: Synthetic peptide
FINPYNDDIQSNERFRG

SEQ ID NO: 75 - protein, 32 aa, synthetic construct; identical to SEQ ID NO: 26
  source 1..32: note = Description of Artificial Sequence: Synthetic polypeptide

SEQ ID NO: 76 - protein, 15 aa, synthetic construct
  source 1..15: note = Description of Artificial Sequence: Synthetic peptide
GAGYNFDGAYRFFDF

SEQ ID NO: 77 - protein, 11 aa, synthetic construct
  source 1..11: note = Description of Artificial Sequence: Synthetic peptide
WGQGTTVTVSS

SEQ ID NO: 78 - protein, 30 aa, synthetic construct; identical to SEQ ID NO: 43
  source 1..30: note = Description of Artificial Sequence: Synthetic polypeptide

SEQ ID NO: 79 - protein, 5 aa, synthetic construct
  source 1..5: note = Description of Artificial Sequence: Synthetic peptide
GYVMH

SEQ ID NO: 80 - protein, 14 aa, synthetic construct
  source 1..14: note = Description of Artificial Sequence: Synthetic peptide
WVRQAPGQGLEWMG

SEQ ID NO: 81 - protein, 17 aa, synthetic construct
  source 1..17: note = Description of Artificial Sequence: Synthetic peptide
FINPYNDDIQSNERFRG

SEQ ID NO: 82 - protein, 32 aa, synthetic construct
  source 1..32: note = Description of Artificial Sequence: Synthetic polypeptide
RVTMTSDKSITTAYMELSRLRSDDTAVYYCAR

SEQ ID NO: 83 - protein, 15 aa, synthetic construct
  source 1..15: note = Description of Artificial Sequence: Synthetic peptide
GAGYNFDGAYRFFDF

SEQ ID NO: 84 - protein, 11 aa, synthetic construct
  source 1..11: note = Description of Artificial Sequence: Synthetic peptide
WGQGTTVTVSS

SEQ ID NO: 85 - protein, 30 aa, synthetic construct
  source 1..30: note = Description of Artificial Sequence: Synthetic polypeptide
QVQLVQSGAEVKKPGSSVKVSCKASGYTFS

SEQ ID NO: 86 - protein, 5 aa, synthetic construct
  source 1..5: note = Description of Artificial Sequence: Synthetic peptide
GYAIS

SEQ ID NO: 87 - protein, 14 aa, synthetic construct
  source 1..14: note = Description of Artificial Sequence: Synthetic peptide
WVRQAPGQGLEWMG

SEQ ID NO: 88 - protein, 17 aa, synthetic construct
  source 1..17: note = Description of Artificial Sequence: Synthetic peptide
FINPYNDDIQSNERFRG

SEQ ID NO: 89 - protein, 32 aa, synthetic construct; identical to SEQ ID NO: 26
  source 1..32: note = Description of Artificial Sequence: Synthetic polypeptide

SEQ ID NO: 90 - protein, 15 aa, synthetic construct
  source 1..15: note = Description of Artificial Sequence: Synthetic peptide
GAGYNFDGAYRFFDF

SEQ ID NO: 91 - protein, 11 aa, synthetic construct
  source 1..11: note = Description of Artificial Sequence: Synthetic peptide
WGQGTLVTVSS

SEQ ID NO: 92 - protein, 30 aa, synthetic construct; identical to SEQ ID NO: 57
  source 1..30: note = Description of Artificial Sequence: Synthetic polypeptide

SEQ ID NO: 93 - protein, 5 aa, synthetic construct
  source 1..5: note = Description of Artificial Sequence: Synthetic peptide
GYVMH

SEQ ID NO: 94 - protein, 14 aa, synthetic construct
  source 1..14: note = Description of Artificial Sequence: Synthetic peptide
WVRQAPGQGLEWMG

SEQ ID NO: 95 - protein, 17 aa, synthetic construct
  source 1..17: note = Description of Artificial Sequence: Synthetic peptide
FIIPIFGTANYAQKFQG

SEQ ID NO: 96 - protein, 32 aa, synthetic construct; identical to SEQ ID NO: 26
  source 1..32: note = Description of Artificial Sequence: Synthetic polypeptide

SEQ ID NO: 97 - protein, 15 aa, synthetic construct
  source 1..15: note = Description of Artificial Sequence: Synthetic peptide
GAGYNFDGAYRFFDF

SEQ ID NO: 98 - protein, 11 aa, synthetic construct
  source 1..11: note = Description of Artificial Sequence: Synthetic peptide
WGQGTLVTVSS

SEQ ID NO: 99 - protein, 30 aa, synthetic construct; identical to SEQ ID NO: 85
  source 1..30: note = Description of Artificial Sequence: Synthetic polypeptide

SEQ ID NO: 100 - protein, 5 aa, synthetic construct
  source 1..5: note = Description of Artificial Sequence: Synthetic peptide
GYAIS

SEQ ID NO: 101 - protein, 14 aa, synthetic construct
  source 1..14: note = Description of Artificial Sequence: Synthetic peptide
WVRQAPGQGLEWMG

SEQ ID NO: 102 - protein, 17 aa, synthetic construct
  source 1..17: note = Description of Artificial Sequence: Synthetic peptide
FIIPIFGTANYAQKFQG

SEQ ID NO: 103 - protein, 32 aa, synthetic construct; identical to SEQ ID NO: 26
  source 1..32: note = Description of Artificial Sequence: Synthetic polypeptide

SEQ ID NO: 104 - protein, 15 aa, synthetic construct
  source 1..15: note = Description of Artificial Sequence: Synthetic peptide
GAGYNFDGAYRFFDF

SEQ ID NO: 105 - protein, 11 aa, synthetic construct
  source 1..11: note = Description of Artificial Sequence: Synthetic peptide
WGQGTLVTVSS

SEQ ID NO: 106 - protein, 23 aa, synthetic construct
  source 1..23: note = Description of Artificial Sequence: Synthetic peptide
DVVMTQSPLSLPVSLGDQASISC

SEQ ID NO: 107 - protein, 16 aa, synthetic construct
  source 1..16: note = Description of Artificial Sequence: Synthetic peptide
RSSQRLVHSNGNTYLH

SEQ ID NO: 108 - protein, 15 aa, synthetic construct
  source 1..15: note = Description of Artificial Sequence: Synthetic peptide
WYLQKPGQSPKLLIY

SEQ ID NO: 109 - protein, 7 aa, synthetic construct
  source 1..7: note = Description of Artificial Sequence: Synthetic peptide
RVSNRFP

SEQ ID NO: 110 - protein, 32 aa, synthetic construct
  source 1..32: note = Description of Artificial Sequence: Synthetic polypeptide
GVPDRFSGSGSGTDFTLKISRVEAEDLGIYFC

SEQ ID NO: 111 - protein, 9 aa, synthetic construct
  source 1..9: note = Description of Artificial Sequence: Synthetic peptide
SQSTHVPYT

SEQ ID NO: 112 - protein, 10 aa, synthetic construct
  source 1..10: note = Description of Artificial Sequence: Synthetic peptide
FGGGTKLEIK

SEQ ID NO: 113 - protein, 23 aa, synthetic construct
  source 1..23: note = Description of Artificial Sequence: Synthetic peptide
DVVMTQSPLSLPVTPGEPASISC

SEQ ID NO: 114 - protein, 16 aa, synthetic construct
  source 1..16: note = Description of Artificial Sequence: Synthetic peptide
RSSQRLVHSNGNTYLH

SEQ ID NO: 115 - protein, 15 aa, synthetic construct
  source 1..15: note = Description of Artificial Sequence: Synthetic peptide
WYLQKPGQSPQLLIY

SEQ ID NO: 116 - protein, 7 aa, synthetic construct
  source 1..7: note = Description of Artificial Sequence: Synthetic peptide
RVSNRFP

SEQ ID NO: 117 - protein, 32 aa, synthetic construct
  source 1..32: note = Description of Artificial Sequence: Synthetic polypeptide
GVPDRFSGSGSGTDFTLKISRVEAEDVGVYFC

SEQ ID NO: 118 - protein, 9 aa, synthetic construct
  source 1..9: note = Description of Artificial Sequence: Synthetic peptide
SQSTHVPYT

SEQ ID NO: 119 - protein, 10 aa, synthetic construct
  source 1..10: note = Description of Artificial Sequence: Synthetic peptide
FGGGTKVEIK

SEQ ID NO: 120 - protein, 23 aa, synthetic construct
  source 1..23: note = Description of Artificial Sequence: Synthetic peptide
EVVMTQSPGTLSLSPGERATLSC

SEQ ID NO: 121 - protein, 16 aa, synthetic construct
  source 1..16: note = Description of Artificial Sequence: Synthetic peptide
RSSQRLVHSNGNTYLH

SEQ ID NO: 122 - protein, 15 aa, synthetic construct
  source 1..15: note = Description of Artificial Sequence: Synthetic peptide
WYQQKPGQAPRLLIY

SEQ ID NO: 123 - protein, 7 aa, synthetic construct
  source 1..7: note = Description of Artificial Sequence: Synthetic peptide
RVSNRFP

SEQ ID NO: 124 - protein, 32 aa, synthetic construct
  source 1..32: note = Description of Artificial Sequence: Synthetic polypeptide
GIPDRFSGSGSGTDFTLTISRLEPEDFAVYFC

SEQ ID NO: 125 - protein, 9 aa, synthetic construct
  source 1..9: note = Description of Artificial Sequence: Synthetic peptide
SQSTHVPYT

SEQ ID NO: 126 - protein, 10 aa, synthetic construct
  source 1..10: note = Description of Artificial Sequence: Synthetic peptide
FGGGTKVEIK

SEQ ID NO: 127 - protein, 23 aa, synthetic construct
  source 1..23: note = Description of Artificial Sequence: Synthetic peptide
DVVMTQSPLSLPVTLGQPASISC

SEQ ID NO: 128 - protein, 16 aa, synthetic construct
  source 1..16: note = Description of Artificial Sequence: Synthetic peptide
RSSQRLVHSNGNTYLH

SEQ ID NO: 129 - protein, 15 aa, synthetic construct
  source 1..15: note = Description of Artificial Sequence: Synthetic peptide
WYQQRPGQSPRLLIY

SEQ ID NO: 130 - protein, 7 aa, synthetic construct
  source 1..7: note = Description of Artificial Sequence: Synthetic peptide
RVSNRFP

SEQ ID NO: 131 - protein, 32 aa, synthetic construct; identical to SEQ ID NO: 117
  source 1..32: note = Description of Artificial Sequence: Synthetic polypeptide

SEQ ID NO: 132 - protein, 9 aa, synthetic construct
  source 1..9: note = Description of Artificial Sequence: Synthetic peptide
SQSTHVPYT

SEQ ID NO: 133 - protein, 10 aa, synthetic construct
  source 1..10: note = Description of Artificial Sequence: Synthetic peptide
FGGGTKVEIK

SEQ ID NO: 134 - protein, 23 aa, synthetic construct
  source 1..23: note = Description of Artificial Sequence: Synthetic peptide
DVVMTQTPLSLPVTPGEPASISC

SEQ ID NO: 135 - protein, 16 aa, synthetic construct
  source 1..16: note = Description of Artificial Sequence: Synthetic peptide
RSSQRLVHSNGNTYLH

SEQ ID NO: 136 - protein, 15 aa, synthetic construct
  source 1..15: note = Description of Artificial Sequence: Synthetic peptide
WYLQKPGQSPQLLIY

SEQ ID NO: 137 - protein, 7 aa, synthetic construct
  source 1..7: note = Description of Artificial Sequence: Synthetic peptide
RVSNRFP

SEQ ID NO: 138 - protein, 32 aa, synthetic construct; identical to SEQ ID NO: 117
  source 1..32: note = Description of Artificial Sequence: Synthetic polypeptide

SEQ ID NO: 139 - protein, 9 aa, synthetic construct
  source 1..9: note = Description of Artificial Sequence: Synthetic peptide
SQSTHVPYT

SEQ ID NO: 140 - protein, 10 aa, synthetic construct
  source 1..10: note = Description of Artificial Sequence: Synthetic peptide
FGGGTKVEIK

SEQ ID NO: 141 - protein, 23 aa, synthetic construct
  source 1..23: note = Description of Artificial Sequence: Synthetic peptide
DVVMTQTPLSLSVTPGQPASISC

SEQ ID NO: 142 - protein, 16 aa, synthetic construct
  source 1..16: note = Description of Artificial Sequence: Synthetic peptide
RSSQRLVHSNGNTYLH

SEQ ID NO: 143 - protein, 15 aa, synthetic construct
  source 1..15: note = Description of Artificial Sequence: Synthetic peptide
WYLQKPGQSPQLLIY

SEQ ID NO: 144 - protein, 7 aa, synthetic construct
  source 1..7: note = Description of Artificial Sequence: Synthetic peptide
RVSNRFP

SEQ ID NO: 145 - protein, 32 aa, synthetic construct; identical to SEQ ID NO: 117
  source 1..32: note = Description of Artificial Sequence: Synthetic polypeptide

SEQ ID NO: 146 - protein, 9 aa, synthetic construct
  source 1..9: note = Description of Artificial Sequence: Synthetic peptide
SQSTHVPYT

SEQ ID NO: 147 - protein, 10 aa, synthetic construct
  source 1..10: note = Description of Artificial Sequence: Synthetic peptide
FGGGTKVEIK

SEQ ID NO: 148 - protein, 23 aa, synthetic construct
  source 1..23: note = Description of Artificial Sequence: Synthetic peptide
DVVMTQSPLSLPVTLGQPASISC

SEQ ID NO: 149 - protein, 15 aa, synthetic construct
  source 1..15: note = Description of Artificial Sequence: Synthetic peptide
RSSQSLVYSDGNTYH

SEQ ID NO: 150 - protein, 15 aa, synthetic construct
  source 1..15: note = Description of Artificial Sequence: Synthetic peptide
WYQQRPGQSPRLLIY

SEQ ID NO: 151 - protein, 7 aa, synthetic construct
  source 1..7: note = Description of Artificial Sequence: Synthetic peptide
RVSNRFP

SEQ ID NO: 152 - protein, 32 aa, synthetic construct; identical to SEQ ID NO: 117
  source 1..32: note = Description of Artificial Sequence: Synthetic polypeptide

SEQ ID NO: 153 - protein, 9 aa, synthetic construct
  source 1..9: note = Description of Artificial Sequence: Synthetic peptide
SQSTHVPYT

SEQ ID NO: 154 - protein, 10 aa, synthetic construct
  source 1..10: note = Description of Artificial Sequence: Synthetic peptide
FGGGTKVEIK

SEQ ID NO: 155 - protein, 23 aa, synthetic construct
  source 1..23: note = Description of Artificial Sequence: Synthetic peptide
DVVMTQSPLSLPVTLGQPASISC

SEQ ID NO: 156 - protein, 16 aa, synthetic construct
  source 1..16: note = Description of Artificial Sequence: Synthetic peptide
RSSQRLVHSNGNTYLH

SEQ ID NO: 157 - protein, 15 aa, synthetic construct
  source 1..15: note = Description of Artificial Sequence: Synthetic peptide
WYQQRPGQSPRLLIY

SEQ ID NO: 158 - protein, 7 aa, synthetic construct
  source 1..7: note = Description of Artificial Sequence: Synthetic peptide
KVSNRDS

SEQ ID NO: 159 - protein, 32 aa, synthetic construct; identical to SEQ ID NO: 117
  source 1..32: note = Description of Artificial Sequence: Synthetic polypeptide

SEQ ID NO: 160 - protein, 9 aa, synthetic construct
  source 1..9: note = Description of Artificial Sequence: Synthetic peptide
SQSTHVPYT

SEQ ID NO: 161 - protein, 10 aa, synthetic construct
  source 1..10: note = Description of Artificial Sequence: Synthetic peptide
FGGGTKVEIK

SEQ ID NO: 162 - protein, 23 aa, synthetic construct
  source 1..23: note = Description of Artificial Sequence: Synthetic peptide
DVVMTQSPLSLPVTLGQPASISC

SEQ ID NO: 163 - protein, 16 aa, synthetic construct
  source 1..16: note = Description of Artificial Sequence: Synthetic peptide
RSSQRLVHSNGNTYLH

SEQ ID NO: 164 - protein, 15 aa, synthetic construct
  source 1..15: note = Description of Artificial Sequence: Synthetic peptide
WYQQRPGQSPRLLIY

SEQ ID NO: 165 - protein, 7 aa, synthetic construct
  source 1..7: note = Description of Artificial Sequence: Synthetic peptide
RVSNRFP

SEQ ID NO: 166 - protein, 32 aa, synthetic construct; identical to SEQ ID NO: 117
  source 1..32: note = Description of Artificial Sequence: Synthetic polypeptide

SEQ ID NO: 167 - protein, 9 aa, synthetic construct
  source 1..9: note = Description of Artificial Sequence: Synthetic peptide
MQSTHWPYT

SEQ ID NO: 168 - protein, 10 aa, synthetic construct
  source 1..10: note = Description of Artificial Sequence: Synthetic peptide
FGGGTKVEIK

SEQ ID NO: 169 - protein, 23 aa, synthetic construct
  source 1..23: note = Description of Artificial Sequence: Synthetic peptide
DVVMTQSPLSLPVTLGQPASISC

SEQ ID NO: 170 - protein, 15 aa, synthetic construct
  source 1..15: note = Description of Artificial Sequence: Synthetic peptide
RSSQSLVYSDGNTYH

SEQ ID NO: 171 - protein, 15 aa, synthetic construct
  source 1..15: note = Description of Artificial Sequence: Synthetic peptide
WYQQRPGQSPRLLIY

SEQ ID NO: 172 - protein, 7 aa, synthetic construct
  source 1..7: note = Description of Artificial Sequence: Synthetic peptide
KVSNRDS

SEQ ID NO: 173 - protein, 32 aa, synthetic construct; identical to SEQ ID NO: 117
  source 1..32: note = Description of Artificial Sequence: Synthetic polypeptide

SEQ ID NO: 174 - protein, 9 aa, synthetic construct
  source 1..9: note = Description of Artificial Sequence: Synthetic peptide
MQSTHWPYT

SEQ ID NO: 175 - protein, 10 aa, synthetic construct
  source 1..10: note = Description of Artificial Sequence: Synthetic peptide
FGGGTKVEIK

SEQ ID NO: 176 - protein, 124 aa, synthetic construct
  source 1..124: note = Description of Artificial Sequence: Synthetic polypeptide
EVRLQQSGPDLIKPGASVKMSCKASGYTFTGYVMHWVKQRPGQGLEWIGFINPYNDDIQSNERFRGKATLTSDKSSTTAYMELSSLTSEDSAVYYCARGAGYNFDGAYRFFDFWGQGTTLTVSS

SEQ ID NO: 177 - protein, 124 aa, synthetic construct
  source 1..124: note = Description of Artificial Sequence: Synthetic polypeptide
QVQLVQSGAEVKKPGASVKVSCKASGYTFTGYVMHWVRQAPGQGLEWMGFINPYNDDIQSNERFRGRVTMTSDKSTTTAYMELSSLRSEDTAVYYCARGAGYNFDGAYRFFDFWGQGTLVTVSS

SEQ ID NO: 178 - protein, 124 aa, synthetic construct
  source 1..124: note = Description of Artificial Sequence: Synthetic polypeptide
QVQLVQSGAEVKKPGASVKVSCKASGYTFTGYVMHWVRQAPGQGLEWMGFINPYNDDIQSNERFRGWVTMTSDKSITTAYMELSRLRSDDTAVYYCARGAGYNFDGAYRFFDFWGQGTLVTVSS

SEQ ID NO: 179 - protein, 124 aa, synthetic construct
  source 1..124: note = Description of Artificial Sequence: Synthetic polypeptide
QVQLVQSGAEVKKPGSSVKVSCKASGYTFTGYVMHWVRQAPGQGLEWMGFINPYNDDIQSNERFRGRVTITSDKSTTTAYMELSSLRSEDTAVYYCARGAGYNFDGAYRFFDFWGQGTLVTVSS

SEQ ID NO: 180 - protein, 124 aa, synthetic construct
  source 1..124: note = Description of Artificial Sequence: Synthetic polypeptide
QVQLVQSGAEVKKPGASVKVSCKASGYTFTGYVMHWVRQAPGQRLEWMGFINPYNDDIQSNERFRGRVTITSDKSATTAYMELSSLRSEDTAVYYCARGAGYNFDGAYRFFDFWGQGTLVTVSS

SEQ ID NO: 181 - protein, 124 aa, synthetic construct
  source 1..124: note = Description of Artificial Sequence: Synthetic polypeptide
QVQLVQSGAEVKKPGSSVKVSCKASGYTFTGYVMHWVRQAPGQGLEWMGFINPYNDDIQSNERFRGRVTITSDKSTTTAYMELSSLRSEDTAVYYCARGAGYNFDGAYRFFDFWGQGTTVTVSS

SEQ ID NO: 182 - protein, 124 aa, synthetic construct
  source 1..124: note = Description of Artificial Sequence: Synthetic polypeptide
QVQLVQSGAEVKKPGASVKVSCKASGYTFTGYVMHWVRQAPGQGLEWMGFINPYNDDIQSNERFRGRVTMTSDKSITTAYMELSRLRSDDTAVYYCARGAGYNFDGAYRFFDFWGQGTTVTVSS

SEQ ID NO: 183 - protein, 124 aa, synthetic construct
  source 1..124: note = Description of Artificial Sequence: Synthetic polypeptide
QVQLVQSGAEVKKPGSSVKVSCKASGYTFSGYAISWVRQAPGQGLEWMGFINPYNDDIQSNERFRGRVTITSDKSTTTAYMELSSLRSEDTAVYYCARGAGYNFDGAYRFFDFWGQGTLVTVSS

SEQ ID NO: 184 - protein, 124 aa, synthetic construct
  source 1..124: note = Description of Artificial Sequence: Synthetic polypeptide
QVQLVQSGAEVKKPGSSVKVSCKASGYTFTGYVMHWVRQAPGQGLEWMGFIIPIFGTANYAQKFQGRVTITSDKSTTTAYMELSSLRSEDTAVYYCARGAGYNFDGAYRFFDFWGQGTLVTVSS

SEQ ID NO: 185 - protein, 124 aa, synthetic construct
  source 1..124: note = Description of Artificial Sequence: Synthetic polypeptide
QVQLVQSGAEVKKPGSSVKVSCKASGYTFSGYAISWVRQAPGQGLEWMGFIIPIFGTANYAQKFQGRVTITSDKSTTTAYMELSSLRSEDTAVYYCARGAGYNFDGAYRFFDFWGQGTLVTVSS

SEQ ID NO: 186 - protein, 112 aa, synthetic construct
  source 1..112: note = Description of Artificial Sequence: Synthetic polypeptide
DVVMTQSPLSLPVSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIK

SEQ ID NO: 187 - protein, 112 aa, synthetic construct
  source 1..112: note = Description of Artificial Sequence: Synthetic polypeptide
DVVMTQSPLSLPVTPGEPASISCRSSQRLVHSNGNTYLHWYLQKPGQSPQLLIYRVSNRFPGVPDRFSGSGSGTDFTLKISRVEAEDVGVYFCSQSTHVPYTFGGGTKVEIK

SEQ ID NO: 188 - protein, 112 aa, synthetic construct
  source 1..112: note = Description of Artificial Sequence: Synthetic polypeptide
EVVMTQSPGTLSLSPGERATLSCRSSQRLVHSNGNTYLHWYQQKPGQAPRLLIYRVSNRFPGIPDRFSGSGSGTDFTLTISRLEPEDFAVYFCSQSTHVPYTFGGGTKVEIK

SEQ ID NO: 189 - protein, 112 aa, synthetic construct
  source 1..112: note = Description of Artificial Sequence: Synthetic polypeptide
DVVMTQSPLSLPVTLGQPASISCRSSQRLVHSNGNTYLHWYQQRPGQSPRLLIYRVSNRFPGVPDRFSGSGSGTDFTLKISRVEAEDVGVYFCSQSTHVPYTFGGGTKVEIK

SEQ ID NO: 190 - protein, 112 aa, synthetic construct
  source 1..112: note = Description of Artificial Sequence: Synthetic polypeptide
DVVMTQTPLSLPVTPGEPASISCRSSQRLVHSNGNTYLHWYLQKPGQSPQLLIYRVSNRFPGVPDRFSGSGSGTDFTLKISRVEAEDVGVYFCSQSTHVPYTFGGGTKVEIK

SEQ ID NO: 191 - protein, 112 aa, synthetic construct
  source 1..112: note = Description of Artificial Sequence: Synthetic polypeptide
DVVMTQTPLSLSVTPGQPASISCRSSQRLVHSNGNTYLHWYLQKPGQSPQLLIYRVSNRFPGVPDRFSGSGSGTDFTLKISRVEAEDVGVYFCSQSTHVPYTFGGGTKVEIK

SEQ ID NO: 192 - protein, 111 aa, synthetic construct
  source 1..111: note = Description of Artificial Sequence: Synthetic polypeptide
DVVMTQSPLSLPVTLGQPASISCRSSQSLVYSDGNTYHWYQQRPGQSPRLLIYRVSNRFPGVPDRFSGSGSGTDFTLKISRVEAEDVGVYFCSQSTHVPYTFGGGTKVEIK

SEQ ID NO: 193 - protein, 112 aa, synthetic construct
  source 1..112: note = Description of Artificial Sequence: Synthetic polypeptide
DVVMTQSPLSLPVTLGQPASISCRSSQRLVHSNGNTYLHWYQQRPGQSPRLLIYKVSNRDSGVPDRFSGSGSGTDFTLKISRVEAEDVGVYFCSQSTHVPYTFGGGTKVEIK

SEQ ID NO: 194 - protein, 112 aa, synthetic construct
  source 1..112: note = Description of Artificial Sequence: Synthetic polypeptide
DVVMTQSPLSLPVTLGQPASISCRSSQRLVHSNGNTYLHWYQQRPGQSPRLLIYRVSNRFPGVPDRFSGSGSGTDFTLKISRVEAEDVGVYFCMQSTHWPYTFGGGTKVEIK

SEQ ID NO: 195 - protein, 111 aa, synthetic construct
  source 1..111: note = Description of Artificial Sequence: Synthetic polypeptide
DVVMTQSPLSLPVTLGQPASISCRSSQSLVYSDGNTYHWYQQRPGQSPRLLIYKVSNRDSGVPDRFSGSGSGTDFTLKISRVEAEDVGVYFCMQSTHWPYTFGGGTKVEIK

SEQ ID NO: 196 - protein, 454 aa, synthetic construct
  source 1..454: note = Description of Artificial Sequence: Synthetic polypeptide
EVRLQQSGPDLIKPGASVKMSCKASGYTFTGYVMHWVKQRPGQGLEWIGFINPYNDDIQSNERFRGKATLTSDKSSTTAYMELSSLTSEDSAVYYCARGAGYNFDGAYRFFDFWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 197 - protein, 454 aa, synthetic construct
  source 1..454: note = Description of Artificial Sequence: Synthetic polypeptide
EVRLQQSGPDLIKPGASVKMSCKASGYTFTGYVMHWVKQRPGQGLEWIGFINPYNDDIQSNERFRGKATLTSDKSSTTAYMELSSLTSEDSAVYYCARGAGYNFDGAYRFFDFWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 198 - protein, 219 aa, synthetic construct
  source 1..219: note = Description of Artificial Sequence: Synthetic polypeptide
DVVMTQSPLSLPVSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 199 - protein, 227 aa, synthetic construct
  source 1..227: note = Description of Artificial Sequence: Synthetic polypeptide
EVRLQQSGPDLIKPGASVKMSCKASGYTFTGYVMHWVKQRPGQGLEWIGFINPYNDDIQSNERFRGKATLTSDKSSTTAYMELSSLTSEDSAVYYCARGAGYNFDGAYRFFDFWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 200 - protein, 474 aa, synthetic construct
  source 1..474: note = Description of Artificial Sequence: Synthetic polypeptide
METDTLLLWVLLLWVPGSTGQVQLVQSGAEVKKPGSSVKVSCKASGYTFTGYVMHWVRQAPGQGLEWMGFINPYNDDIQSNERFRGRVTITSDKSTTTAYMELSSLRSEDTAVYYCARGAGYNFDGAYRFFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 201 - protein, 474 aa, synthetic construct
  source 1..474: note = Description of Artificial Sequence: Synthetic polypeptide
METDTLLLWVLLLWVPGSTGQVQLVQSGAEVKKPGSSVKVSCKASGYTFTGYVMHWVRQAPGQGLEWMGFINPYNDDIQSNERFRGRVTITSDKSTTTAYMELSSLRSEDTAVYYCARGAGYNFDGAYRFFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 202 - protein, 239 aa, synthetic construct
  source 1..239: note = Description of Artificial Sequence: Synthetic polypeptide
METDTLLLWVLLLWVPGSTGDVVMTQSPLSLPVTLGQPASISCRSSQRLVHSNGNTYLHWYQQRPGQSPRLLIYRVSNRFPGVPDRFSGSGSGTDFTLKISRVEAEDVGVYFCSQSTHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 203 - protein, 454 aa, synthetic construct
  source 1..454: note = Description of Artificial Sequence: Synthetic polypeptide
QVQLVQSGAEVKKPGSSVKVSCKASGYTFTGYVMHWVRQAPGQGLEWMGFINPYNDDIQSNERFRGRVTITSDKSTTTAYMELSSLRSEDTAVYYCARGAGYNFDGAYRFFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 204 - protein, 219 aa, synthetic construct
  source 1..219: note = Description of Artificial Sequence: Synthetic polypeptide
DVVMTQSPLSLPVTLGQPASISCRSSQRLVHSNGNTYLHWYQQRPGQSPRLLIYRVSNRFPGVPDRFSGSGSGTDFTLKISRVEAEDVGVYFCSQSTHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 205 - protein, 454 aa, synthetic construct
  source 1..454: note = Description of Artificial Sequence: Synthetic polypeptide
QVQLVQSGAEVKKPGSSVKVSCKASGYTFTGYVMHWVRQAPGQGLEWMGFIIPIFGTANYAQKFQGRVTITSDKSTTTAYMELSSLRSEDTAVYYCARGAGYNFDGAYRFFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 206 - protein, 219 aa, synthetic construct; identical to SEQ ID NO: 204
  source 1..219: note = Description of Artificial Sequence: Synthetic polypeptide

SEQ ID NO: 207 - protein, 454 aa, synthetic construct; identical to SEQ ID NO: 203
  source 1..454: note = Description of Artificial Sequence: Synthetic polypeptide

SEQ ID NO: 208 - protein, 219 aa, synthetic construct; identical to SEQ ID NO: 204
  source 1..219: note = Description of Artificial Sequence: Synthetic polypeptide

SEQ ID NO: 209 - protein, 454 aa, synthetic construct; identical to SEQ ID NO: 205
  source 1..454: note = Description of Artificial Sequence: Synthetic polypeptide

SEQ ID NO: 210 - protein, 219 aa, synthetic construct; identical to SEQ ID NO: 204
  source 1..219: note = Description of Artificial Sequence: Synthetic polypeptide

SEQ ID NO: 211 - protein, 454 aa, synthetic construct; identical to SEQ ID NO: 203
  source 1..454: note = Description of Artificial Sequence: Synthetic polypeptide

SEQ ID NO: 212 - protein, 219 aa, synthetic construct; identical to SEQ ID NO: 204
  source 1..219: note = Description of Artificial Sequence: Synthetic polypeptide

SEQ ID NO: 213 - protein, 5 aa, synthetic construct
  source 1..5: note = Description of Artificial Sequence: Synthetic peptide
GGGGS

SEQ ID NO: 214 - protein, 7 aa, synthetic construct
  source 1..7: note = Description of Artificial Sequence: Synthetic peptide
GGGSSGS

SEQ ID NO: 215 - protein, 11 aa, synthetic construct
  source 1..11: note = Description of Artificial Sequence: Synthetic peptide
SGSSGGSSSGA

SEQ ID NO: 216 - protein, 45 aa, synthetic construct
  source 1..45: note = Description of Artificial Sequence: Synthetic polypeptide
TTTPAPRPPTPAPTIASQPLSLRPEACRPAAGGAVHTRGLDFACD

SEQ ID NO: 217 - protein, 24 aa, synthetic construct
  source 1..24: note = Description of Artificial Sequence: Synthetic peptide
IYIWAPLAGTCGVLLLSLVITLYC

SEQ ID NO: 218 - protein, 112 aa, synthetic construct
  source 1..112: note = Description of Artificial Sequence: Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 219 - protein, 42 aa, synthetic construct
  source 1..42: note = Description of Artificial Sequence: Synthetic polypeptide
KRGRKKLLYIFKQPFMRPVQTTQEEDGCSCRFPEEEEGGCEL

SEQ ID NO: 220 - protein, 41 aa, synthetic construct
  source 1..41: note = Description of Artificial Sequence: Synthetic polypeptide
RSKRSRLLHSDYMNMTPRRPGPTRKHYQPYAPPRDFAAYRS

SEQ ID NO: 221 - protein, 30 aa, synthetic construct
  source 1..30: note = Description of Artificial Sequence: Synthetic polypeptide
QVQLVQSGAEVKKPGASVKVSCKASGYTFP

SEQ ID NO: 222 - protein, 5 aa, synthetic construct
  source 1..5: note = Description of Artificial Sequence: Synthetic peptide
GFVMH

SEQ ID NO: 223 - protein, 14 aa, synthetic construct
  source 1..14: note = Description of Artificial Sequence: Synthetic peptide
WVRQAPGQGLEWMG

SEQ ID NO: 224 - protein, 17 aa, synthetic construct
  source 1..17: note = Description of Artificial Sequence: Synthetic peptide
FINPYNDDIQSNERFRG

SEQ ID NO: 225 - protein, 32 aa, synthetic construct; identical to SEQ ID NO: 12
  source 1..32: note = Description of Artificial Sequence: Synthetic polypeptide

SEQ ID NO: 226 - protein, 15 aa, synthetic construct
  source 1..15: note = Description of Artificial Sequence: Synthetic peptide
GNGMWFDGAYRFFDF

SEQ ID NO: 227 - protein, 11 aa, synthetic construct
  source 1..11: note = Description of Artificial Sequence: Synthetic peptide
WGQGTLVTVSS

SEQ ID NO: 228 - protein, 30 aa, synthetic construct
  source 1..30: note = Description of Artificial Sequence: Synthetic polypeptide
QVQLVQSGAEVKKPGASVKVSCKASGYPFH

SEQ ID NO: 229 - protein, 5 aa, synthetic construct
  source 1..5: note = Description of Artificial Sequence: Synthetic peptide
GYVMH

SEQ ID NO: 230 - protein, 14 aa, synthetic construct
  source 1..14: note = Description of Artificial Sequence: Synthetic peptide
WVRQAPGQGLEWMG

SEQ ID NO: 231 - protein, 17 aa, synthetic construct
  source 1..17: note = Description of Artificial Sequence: Synthetic peptide
FINPYNDDIQSNERFRG

SEQ ID NO: 232 - protein, 32 aa, synthetic construct; identical to SEQ ID NO: 12
  source 1..32: note = Description of Artificial Sequence: Synthetic polypeptide

SEQ ID NO: 233 - protein, 15 aa, synthetic construct
  source 1..15: note = Description of Artificial Sequence: Synthetic peptide
GNGKNFDGAYRFFDF

SEQ ID NO: 234 - protein, 11 aa, synthetic construct
  source 1..11: note = Description of Artificial Sequence: Synthetic peptide
WGQGTLVTVSS

SEQ ID NO: 235 - protein, 30 aa, synthetic construct
  source 1..30: note = Description of Artificial Sequence: Synthetic polypeptide
QVQLVQSGAEVKKPGASVKVSCKASGYTYP

SEQ ID NO: 236 - protein, 5 aa, synthetic construct
  source 1..5: note = Description of Artificial Sequence: Synthetic peptide
GYVMH

SEQ ID NO: 237 - protein, 14 aa, synthetic construct
  source 1..14: note = Description of Artificial Sequence: Synthetic peptide
WVRQAPGQGLEWMG

SEQ ID NO: 238 - protein, 17 aa, synthetic construct
  source 1..17: note = Description of Artificial Sequence: Synthetic peptide
FINPYNDDIQSNERFRG

SEQ ID NO: 239 - protein, 32 aa, synthetic construct; identical to SEQ ID NO: 12
  source 1..32: note = Description of Artificial Sequence: Synthetic polypeptide

SEQ ID NO: 240 - protein, 15 aa, synthetic construct
  source 1..15: note = Description of Artificial Sequence: Synthetic peptide
GNGKWFDGAYRFFDF

SEQ ID NO: 241 - protein, 11 aa, synthetic construct
  source 1..11: note = Description of Artificial Sequence: Synthetic peptide
WGQGTLVTVSS

SEQ ID NO: 242 - protein, 30 aa, synthetic construct
  source 1..30: note = Description of Artificial Sequence: Synthetic polypeptide
QVQLVQSGAEVKKPGASVKVSCKASNQNFH

SEQ ID NO: 243 - protein, 5 aa, synthetic construct
  source 1..5: note = Description of Artificial Sequence: Synthetic peptide
GYHMH

SEQ ID NO: 244 - protein, 14 aa, synthetic construct
  source 1..14: note = Description of Artificial Sequence: Synthetic peptide
WVRQAPGQGLEWMG

SEQ ID NO: 245 - protein, 17 aa, synthetic construct
  source 1..17: note = Description of Artificial Sequence: Synthetic peptide
FINPYNDDIQSNERFRG

SEQ ID NO: 246 - protein, 32 aa, synthetic construct; identical to SEQ ID NO: 12
  source 1..32: note = Description of Artificial Sequence: Synthetic polypeptide

SEQ ID NO: 247 - protein, 15 aa, synthetic construct
  source 1..15: note = Description of Artificial Sequence: Synthetic peptide
GNGKWGDGAYRFFDF

SEQ ID NO: 248 - protein, 11 aa, synthetic construct
  source 1..11: note = Description of Artificial Sequence: Synthetic peptide
WGQGTLVTVSS

SEQ ID NO: 249 - protein, 30 aa, synthetic construct
  source 1..30: note = Description of Artificial Sequence: Synthetic polypeptide
QVQLVQSGAEVKKPGASVKVSCKASSQNFH

SEQ ID NO: 250 - protein, 5 aa, synthetic construct
  source 1..5: note = Description of Artificial Sequence: Synthetic peptide
GFYMH

SEQ ID NO: 251 - protein, 14 aa, synthetic construct
  source 1..14: note = Description of Artificial Sequence: Synthetic peptide
WVRQAPGQGLEWMG

SEQ ID NO: 252 - protein, 17 aa, synthetic construct
  source 1..17: note = Description of Artificial Sequence: Synthetic peptide
FINPYNDDIQSNERFRG

SEQ ID NO: 253 - protein, 32 aa, synthetic construct; identical to SEQ ID NO: 12
  source 1..32: note = Description of Artificial Sequence: Synthetic polypeptide

SEQ ID NO: 254 - protein, 15 aa, synthetic construct
  source 1..15: note = Description of Artificial Sequence: Synthetic peptide
GNGKWGDGAYRFFDF

SEQ ID NO: 255 - protein, 11 aa, synthetic construct
  source 1..11: note = Description of Artificial Sequence: Synthetic peptide
WGQGTLVTVSS

SEQ ID NO: 256 - protein, 30 aa, synthetic construct
  source 1..30: note = Description of Artificial Sequence: Synthetic polypeptide
QVQLVQSGAEVKKPGASVKVSCKASYQDFH

SEQ ID NO: 257 - protein, 5 aa, synthetic construct
  source 1..5: note = Description of Artificial Sequence: Synthetic peptide
GYKMH

SEQ ID NO: 258 - protein, 14 aa, synthetic construct
  source 1..14: note = Description of Artificial Sequence: Synthetic peptide
WVRQAPGQGLEWMG

SEQ ID NO: 259 - protein, 17 aa, synthetic construct
  source 1..17: note = Description of Artificial Sequence: Synthetic peptide
FINPYNDDIQSNERFRG

SEQ ID NO: 260 - protein, 32 aa, synthetic construct; identical to SEQ ID NO: 12
  source 1..32: note = Description of Artificial Sequence: Synthetic polypeptide

SEQ ID NO: 261 - protein, 15 aa, synthetic construct
  source 1..15: note = Description of Artificial Sequence: Synthetic peptide
GNGKWGDGAYRFFDF

SEQ ID NO: 262 - protein, 11 aa, synthetic construct
  source 1..11: note = Description of Artificial Sequence: Synthetic peptide
WGQGTLVTVSS

SEQ ID NO: 263 - protein, 30 aa, synthetic construct
  source 1..30: note = Description of Artificial Sequence: Synthetic polypeptide
QVQLVQSGAEVKKPGASVKVSCKASGYNFH

SEQ ID NO: 264 - protein, 5 aa, synthetic construct
  source 1..5: note = Description of Artificial Sequence: Synthetic peptide
GFYMH

SEQ ID NO: 265 - protein, 14 aa, synthetic construct
  source 1..14: note = Description of Artificial Sequence: Synthetic peptide
WVRQAPGQGLEWMG

SEQ ID NO: 266 - protein, 17 aa, synthetic construct
  source 1..17: note = Description of Artificial Sequence: Synthetic peptide
FINPYNDDIQSNERFRG

SEQ ID NO: 267 - protein, 32 aa, synthetic construct; identical to SEQ ID NO: 12
  source 1..32: note = Description of Artificial Sequence: Synthetic polypeptide

SEQ ID NO: 268 - protein, 15 aa, synthetic construct
  source 1..15: note = Description of Artificial Sequence: Synthetic peptide
GNGKWGDGAYRFFDF

SEQ ID NO: 269 - protein, 11 aa, synthetic construct
  source 1..11: note = Description of Artificial Sequence: Synthetic peptide
WGQGTLVTVSS

SEQ ID NO: 270 - protein, 30 aa, synthetic construct
  source 1..30: note = Description of Artificial Sequence: Synthetic polypeptide
QVQLVQSGAEVKKPGASVKVSCKASTSGFH

SEQ ID NO: 271 - protein, 5 aa, synthetic construct
  source 1..5: note = Description of Artificial Sequence: Synthetic peptide
GYPMH

SEQ ID NO: 272 - protein, 14 aa, synthetic construct
  source 1..14: note = Description of Artificial Sequence: Synthetic peptide
WVRQAPGQGLEWMG

SEQ ID NO: 273 - protein, 17 aa, synthetic construct
  source 1..17: note = Description of Artificial Sequence: Synthetic peptide
FINPYNDDIQSNERFRG

SEQ ID NO: 274 - protein, 32 aa, synthetic construct; identical to SEQ ID NO: 12
  source 1..32: note = Description of Artificial Sequence: Synthetic polypeptide

SEQ ID NO: 275 - protein, 15 aa, synthetic construct
  source 1..15: note = Description of Artificial Sequence: Synthetic peptide
GNGKWGDGAYRFFDF

SEQ ID NO: 276 - protein, 11 aa, synthetic construct
  source 1..11: note = Description of Artificial Sequence: Synthetic peptide
WGQGTLVTVSS

SEQ ID NO: 277 - protein, 30 aa, synthetic construct
  source 1..30: note = Description of Artificial Sequence: Synthetic polypeptide
QVQLVQSGAEVKKPGASVKVSCKASPRGFH

SEQ ID NO: 278 - protein, 5 aa, synthetic construct
  source 1..5: note = Description of Artificial Sequence: Synthetic peptide
GYHMH

SEQ ID NO: 279 - protein, 14 aa, synthetic construct
  source 1..14: note = Description of Artificial Sequence: Synthetic peptide
WVRQAPGQGLEWMG

SEQ ID NO: 280 - protein, 17 aa, synthetic construct
  source 1..17: note = Description of Artificial Sequence: Synthetic peptide
FINPYNDDIQSNERFRG

SEQ ID NO: 281 - protein, 32 aa, synthetic construct; identical to SEQ ID NO: 12
  source 1..32: note = Description of Artificial Sequence: Synthetic polypeptide

SEQ ID NO: 282 - protein, 15 aa, synthetic construct
  source 1..15: note = Description of Artificial Sequence: Synthetic peptide
GNGKWGDGAYRFFDF

SEQ ID NO: 283 - protein, 11 aa, synthetic construct
  source 1..11: note = Description of Artificial Sequence: Synthetic peptide
WGQGTLVTVSS

SEQ ID NO: 284 - protein, 30 aa, synthetic construct
  source 1..30: note = Description of Artificial Sequence: Synthetic polypeptide
QVQLVQSGAEVKKPGASVKVSCKASFQDFH

SEQ ID NO: 285 - protein, 5 aa, synthetic construct
  source 1..5: note = Description of Artificial Sequence: Synthetic peptide
GYAMH

SEQ ID NO: 286 - protein, 14 aa, synthetic construct
  source 1..14: note = Description of Artificial Sequence: Synthetic peptide
WVRQAPGQGLEWMG

SEQ ID NO: 287 - protein, 17 aa, synthetic construct
  source 1..17: note = Description of Artificial Sequence: Synthetic peptide
FINPYNDDIQSNERFRG

SEQ ID NO: 288 - protein, 32 aa, synthetic construct; identical to SEQ ID NO: 12
  source 1..32: note = Description of Artificial Sequence: Synthetic polypeptide

SEQ ID NO: 289 - protein, 15 aa, synthetic construct
  source 1..15: note = Description of Artificial Sequence: Synthetic peptide
GNGKWGDGAYRFFDF

SEQ ID NO: 290 - protein, 11 aa, synthetic construct
  source 1..11: note = Description of Artificial Sequence: Synthetic peptide
WGQGTLVTVSS

SEQ ID NO: 291 - protein, 30 aa, synthetic construct
  source 1..30: note = Description of Artificial Sequence: Synthetic polypeptide
QVQLVQSGAEVKKPGASVKVSCKASSKDFH

SEQ ID NO: 292 - protein, 5 aa, synthetic construct
  source 1..5: note = Description of Artificial Sequence: Synthetic peptide
GFAMH

SEQ ID NO: 293 - protein, 14 aa, synthetic construct
  source 1..14: note = Description of Artificial Sequence: Synthetic peptide
WVRQAPGQGLEWMG

SEQ ID NO: 294 - protein, 17 aa, synthetic construct
  source 1..17: note = Description of Artificial Sequence: Synthetic peptide
FINPYNDDIQSNERFRG

SEQ ID NO: 295 - protein, 32 aa, synthetic construct; identical to SEQ ID NO: 12
  source 1..32: note = Description of Artificial Sequence: Synthetic polypeptide

SEQ ID NO: 296 - protein, 15 aa, synthetic construct
  source 1..15: note = Description of Artificial Sequence: Synthetic peptide
GNGKWGDGAYRFFDF

SEQ ID NO: 297 - protein, 11 aa, synthetic construct
  source 1..11: note = Description of Artificial Sequence: Synthetic peptide
WGQGTLVTVSS

SEQ ID NO: 298 - protein, 30 aa, synthetic construct
  source 1..30: note = Description of Artificial Sequence: Synthetic polypeptide
QVQLVQSGAEVKKPGASVKVSCKASPKGFH

SEQ ID NO: 299 - protein, 5 aa, synthetic construct
  source 1..5: note = Description of Artificial Sequence: Synthetic peptide
GYHMH

SEQ ID NO: 300 - protein, 14 aa, synthetic construct
  source 1..14: note = Description of Artificial Sequence: Synthetic peptide
WVRQAPGQGLEWMG

SEQ ID NO: 301 - protein, 17 aa, synthetic construct
  source 1..17: note = Description of Artificial Sequence: Synthetic peptide
FINPYNDDIQSNERFRG

SEQ ID NO: 302 - protein, 32 aa, synthetic construct; identical to SEQ ID NO: 12
  source 1..32: note = Description of Artificial Sequence: Synthetic polypeptide

SEQ ID NO: 303 - protein, 15 aa, synthetic construct
  source 1..15: note = Description of Artificial Sequence: Synthetic peptide
GNGKWGDGAYRFFDF

SEQ ID NO: 304 - protein, 11 aa, synthetic construct
  source 1..11: note = Description of Artificial Sequence: Synthetic peptide
WGQGTLVTVSS

SEQ ID NO: 305 - skipped

SEQ ID NO: 306 - protein, 17 aa, synthetic construct
  source 1..17: note = Description of Artificial Sequence: Synthetic peptide
FINPYNDDIQSNERFRG

SEQ ID NO: 307 - protein, 15 aa, synthetic construct
  source 1..15: note = Description of Artificial Sequence: Synthetic peptide
  VARIANT 4: note = MOD_RES - K or M
  VARIANT 5: note = MOD_RES - W or N
  VARIANT 6: note = MOD_RES - G or F
GNGXXXDGAYRFFDF

SEQ ID NO: 308 - protein, 23 aa, synthetic construct
  source 1..23: note = Description of Artificial Sequence: Synthetic peptide
DVVMTQSPLSLPVTPGEPASISC

SEQ ID NO: 309 - protein, 16 aa, synthetic construct
  source 1..16: note = Description of Artificial Sequence: Synthetic peptide
RSSQNLVHSNGRTYLH

SEQ ID NO: 310 - protein, 15 aa, synthetic construct
  source 1..15: note = Description of Artificial Sequence: Synthetic peptide
WYLQKPGQSPQLLIY

SEQ ID NO: 311 - protein, 7 aa, synthetic construct
  source 1..7: note = Description of Artificial Sequence: Synthetic peptide
RVSNRFP

SEQ ID NO: 312 - protein, 32 aa, synthetic construct; identical to SEQ ID NO: 117
  source 1..32: note = Description of Artificial Sequence: Synthetic polypeptide

SEQ ID NO: 313 - protein, 9 aa, synthetic construct
  source 1..9: note = Description of Artificial Sequence: Synthetic peptide
SQSTHVPYT

SEQ ID NO: 314 - protein, 10 aa, synthetic construct
  source 1..10: note = Description of Artificial Sequence: Synthetic peptide
FGGGTKVEIK

SEQ ID NO: 315 - protein, 23 aa, synthetic construct
  source 1..23: note = Description of Artificial Sequence: Synthetic peptide
DVVMTQSPLSLPVTPGEPASISC

SEQ ID NO: 316 - protein, 16 aa, synthetic construct
  source 1..16: note = Description of Artificial Sequence: Synthetic peptide
RSSQNLVHSNGRTYLQ

SEQ ID NO: 317 - protein, 15 aa, synthetic construct
  source 1..15: note = Description of Artificial Sequence: Synthetic peptide
WYLQKPGQSPQLLIY

SEQ ID NO: 318 - protein, 7 aa, synthetic construct
  source 1..7: note = Description of Artificial Sequence: Synthetic peptide
RVSNRFP

SEQ ID NO: 319 - protein, 32 aa, synthetic construct; identical to SEQ ID NO: 117
  source 1..32: note = Description of Artificial Sequence: Synthetic polypeptide

SEQ ID NO: 320 - protein, 9 aa, synthetic construct
  source 1..9: note = Description of Artificial Sequence: Synthetic peptide
SQSTHVPYT

SEQ ID NO: 321 - protein, 10 aa, synthetic construct
  source 1..10: note = Description of Artificial Sequence: Synthetic peptide
FGGGTKVEIK

SEQ ID NO: 322 - protein, 16 aa, synthetic construct
  source 1..16: note = Description of Artificial Sequence: Synthetic peptide
  VARIANT 16: note = MOD_RES - Q or H
RSSQNLVHSNGRTYLX

SEQ ID NO: 323 - protein, 7 aa, synthetic construct
  source 1..7: note = Description of Artificial Sequence: Synthetic peptide
RVSNRFP

SEQ ID NO: 324 - protein, 9 aa, synthetic construct
  source 1..9: note = Description of Artificial Sequence: Synthetic peptide
SQSTHVPYT

SEQ ID NO: 325 - protein, 124 aa, synthetic construct
  source 1..124: note = Description of Artificial Sequence: Synthetic polypeptide
QVQLVQSGAEVKKPGASVKVSCKASGYTFPGFVMHWVRQAPGQGLEWMGFINPYNDDIQSNERFRGRVTMTSDKSTTTAYMELSSLRSEDTAVYYCARGNGMWFDGAYRFFDFWGQGTLVTVSS

SEQ ID NO: 326 - protein, 124 aa, synthetic construct
  source 1..124: note = Description of Artificial Sequence: Synthetic polypeptide
QVQLVQSGAEVKKPGASVKVSCKASGYPFHGYVMHWVRQAPGQGLEWMGFINPYNDDIQSNERFRGRVTMTSDKSTTTAYMELSSLRSEDTAVYYCARGNGKNFDGAYRFFDFWGQGTLVTVSS

SEQ ID NO: 327 - protein, 124 aa, synthetic construct
  source 1..124: note = Description of Artificial Sequence: Synthetic polypeptide
QVQLVQSGAEVKKPGASVKVSCKASGYTYPGYVMHWVRQAPGQGLEWMGFINPYNDDIQSNERFRGRVTMTSDKSTTTAYMELSSLRSEDTAVYYCARGNGKWFDGAYRFFDFWGQGTLVTVSS

SEQ ID NO: 328 - protein, 124 aa, synthetic construct
  source 1..124: note = Description of Artificial Sequence: Synthetic polypeptide
QVQLVQSGAEVKKPGASVKVSCKASNQNFHGYHMHWVRQAPGQGLEWMGFINPYNDDIQSNERFRGRVTMTSDKSTTTAYMELSSLRSEDTAVYYCARGNGKWGDGAYRFFDFWGQGTLVTVSS

SEQ ID NO: 329 - protein, 124 aa, synthetic construct
  source 1..124: note = Description of Artificial Sequence: Synthetic polypeptide
QVQLVQSGAEVKKPGASVKVSCKASSQNFHGFYMHWVRQAPGQGLEWMGFINPYNDDIQSNERFRGRVTMTSDKSTTTAYMELSSLRSEDTAVYYCARGNGKWGDGAYRFFDFWGQGTLVTVSS

SEQ ID NO: 330 - protein, 124 aa, synthetic construct
  source 1..124: note = Description of Artificial Sequence: Synthetic polypeptide
QVQLVQSGAEVKKPGASVKVSCKASYQDFHGYKMHWVRQAPGQGLEWMGFINPYNDDIQSNERFRGRVTMTSDKSTTTAYMELSSLRSEDTAVYYCARGNGKWGDGAYRFFDFWGQGTLVTVSS

SEQ ID NO: 331 - protein, 124 aa, synthetic construct
  source 1..124: note = Description of Artificial Sequence: Synthetic polypeptide
QVQLVQSGAEVKKPGASVKVSCKASGYNFHGFYMHWVRQAPGQGLEWMGFINPYNDDIQSNERFRGRVTMTSDKSTTTAYMELSSLRSEDTAVYYCARGNGKWGDGAYRFFDFWGQGTLVTVSS

SEQ ID NO: 332 - protein, 124 aa, synthetic construct
  source 1..124: note = Description of Artificial Sequence: Synthetic polypeptide
QVQLVQSGAEVKKPGASVKVSCKASTSGFHGYPMHWVRQAPGQGLEWMGFINPYNDDIQSNERFRGRVTMTSDKSTTTAYMELSSLRSEDTAVYYCARGNGKWGDGAYRFFDFWGQGTLVTVSS

SEQ ID NO: 333 - protein, 124 aa, synthetic construct
  source 1..124: note = Description of Artificial Sequence: Synthetic polypeptide
QVQLVQSGAEVKKPGASVKVSCKASPRGFHGYHMHWVRQAPGQGLEWMGFINPYNDDIQSNERFRGRVTMTSDKSTTTAYMELSSLRSEDTAVYYCARGNGKWGDGAYRFFDFWGQGTLVTVSS

SEQ ID NO: 334 - protein, 124 aa, synthetic construct
  source 1..124: note = Description of Artificial Sequence: Synthetic polypeptide
QVQLVQSGAEVKKPGASVKVSCKASFQDFHGYAMHWVRQAPGQGLEWMGFINPYNDDIQSNERFRGRVTMTSDKSTTTAYMELSSLRSEDTAVYYCARGNGKWGDGAYRFFDFWGQGTLVTVSS

SEQ ID NO: 335 - protein, 124 aa, synthetic construct
  source 1..124: note = Description of Artificial Sequence: Synthetic polypeptide
QVQLVQSGAEVKKPGASVKVSCKASSKDFHGFAMHWVRQAPGQGLEWMGFINPYNDDIQSNERFRGRVTMTSDKSTTTAYMELSSLRSEDTAVYYCARGNGKWGDGAYRFFDFWGQGTLVTVSS

SEQ ID NO: 336 - protein, 124 aa, synthetic construct
  source 1..124: note = Description of Artificial Sequence: Synthetic polypeptide
QVQLVQSGAEVKKPGASVKVSCKASPKGFHGYHMHWVRQAPGQGLEWMGFINPYNDDIQSNERFRGRVTMTSDKSTTTAYMELSSLRSEDTAVYYCARGNGKWGDGAYRFFDFWGQGTLVTVSS

SEQ ID NO: 337 - protein, 112 aa, synthetic construct
  source 1..112: note = Description of Artificial Sequence: Synthetic polypeptide
DVVMTQSPLSLPVTPGEPASISCRSSQNLVHSNGRTYLHWYLQKPGQSPQLLIYRVSNRFPGVPDRFSGSGSGTDFTLKISRVEAEDVGVYFCSQSTHVPYTFGGGTKVEIK

SEQ ID NO: 338 - protein, 112 aa, synthetic construct
  source 1..112: note = Description of Artificial Sequence: Synthetic polypeptide
DVVMTQSPLSLPVTPGEPASISCRSSQNLVHSNGRTYLQWYLQKPGQSPQLLIYRVSNRFPGVPDRFSGSGSGTDFTLKISRVEAEDVGVYFCSQSTHVPYTFGGGTKVEIK

SEQ ID NO: 339 - protein, 256 aa, synthetic construct
  source 1..256: note = Description of Artificial Sequence: Synthetic polypeptide
QVQLVQSGAEVKKPGASVKVSCKASTSGFHGYPMHWVRQAPGQGLEWMGFINPYNDDIQSNERFRGRVTMTSDKSTTTAYMELSSLRSEDTAVYYCARGNGKWGDGAYRFFDFWGQGTLVTVSSGGGGSGGGGSGGGGSGGGGSDVVMTQSPLSLPVTPGEPASISCRSSQNLVHSNGRTYLQWYLQKPGQSPQLLIYRVSNRFPGVPDRFSGSGSGTDFTLKISRVEAEDVGVYFCSQSTHVPYTFGGGTKVEIK

SEQ ID NO: 340 - protein, 256 aa, synthetic construct
  source 1..256: note = Description of Artificial Sequence: Synthetic polypeptide
QVQLVQSGAEVKKPGASVKVSCKASPRGFHGYHMHWVRQAPGQGLEWMGFINPYNDDIQSNERFRGRVTMTSDKSTTTAYMELSSLRSEDTAVYYCARGNGKWGDGAYRFFDFWGQGTLVTVSSGGGGSGGGGSGGGGSGGGGSDVVMTQSPLSLPVTPGEPASISCRSSQNLVHSNGRTYLQWYLQKPGQSPQLLIYRVSNRFPGVPDRFSGSGSGTDFTLKISRVEAEDVGVYFCSQSTHVPYTFGGGTKVEIK

SEQ ID NO: 341 - protein, 256 aa, synthetic construct
  source 1..256: note = Description of Artificial Sequence: Synthetic polypeptide
QVQLVQSGAEVKKPGASVKVSCKASFQDFHGYAMHWVRQAPGQGLEWMGFINPYNDDIQSNERFRGRVTMTSDKSTTTAYMELSSLRSEDTAVYYCARGNGKWGDGAYRFFDFWGQGTLVTVSSGGGGSGGGGSGGGGSGGGGSDVVMTQSPLSLPVTPGEPASISCRSSQNLVHSNGRTYLQWYLQKPGQSPQLLIYRVSNRFPGVPDRFSGSGSGTDFTLKISRVEAEDVGVYFCSQSTHVPYTFGGGTKVEIK

SEQ ID NO: 342 - protein, 256 aa, synthetic construct
  source 1..256: note = Description of Artificial Sequence: Synthetic polypeptide
QVQLVQSGAEVKKPGASVKVSCKASSKDFHGFAMHWVRQAPGQGLEWMGFINPYNDDIQSNERFRGRVTMTSDKSTTTAYMELSSLRSEDTAVYYCARGNGKWGDGAYRFFDFWGQGTLVTVSSGGGGSGGGGSGGGGSGGGGSDVVMTQSPLSLPVTPGEPASISCRSSQNLVHSNGRTYLQWYLQKPGQSPQLLIYRVSNRFPGVPDRFSGSGSGTDFTLKISRVEAEDVGVYFCSQSTHVPYTFGGGTKVEIK

SEQ ID NO: 343 - protein, 256 aa, synthetic construct
  source 1..256: note = Description of Artificial Sequence: Synthetic polypeptide
QVQLVQSGAEVKKPGASVKVSCKASPKGFHGYHMHWVRQAPGQGLEWMGFINPYNDDIQSNERFRGRVTMTSDKSTTTAYMELSSLRSEDTAVYYCARGNGKWGDGAYRFFDFWGQGTLVTVSSGGGGSGGGGSGGGGSGGGGSDVVMTQSPLSLPVTPGEPASISCRSSQNLVHSNGRTYLQWYLQKPGQSPQLLIYRVSNRFPGVPDRFSGSGSGTDFTLKISRVEAEDVGVYFCSQSTHVPYTFGGGTKVEIK

SEQ ID NO: 344 - protein, 4 aa, synthetic construct
  source 1..4: note = Description of Artificial Sequence: Synthetic peptide
  REGION 1..4: note = See specification as filed for detailed description of substitutions and preferred embodiments
GGGS

SEQ ID NO: 345 - protein, 20 aa, synthetic construct
  source 1..20: note = Description of Artificial Sequence: Synthetic peptide
GGGGSGGGGSGGGGSGGGGS

SEQ ID NO: 346 - protein, 15 aa, synthetic construct
  source 1..15: note = Description of Artificial Sequence: Synthetic peptide
GGGGSGGGGSGGGGS

SEQ ID NO: 347 - protein, 4 aa, synthetic construct
  source 1..4: note = Description of Artificial Sequence: Synthetic peptide
GGGS

(19) United States
(12) Patent Application Publication
(10) Pub. No.: US 2023/0192848 A1
(43) Pub. Date: Jun. 22, 2023

(54) ENGINEERED CELL COMPOSITIONS AND METHODS OF USE THEREOF

(71) Applicant: Marengo Therapeutics, Inc., Cambridge, MA (US)

(72) Inventor: Andreas Loew, Boston, MA (US)

(21) Appl. No.: 17/932,416

(22) Filed: Sep. 15, 2022

Related U.S. Application Data
(63) Continuation of application No. PCT/US21/22408, filed on Mar. 15, 2021.
(60) Provisional application No. 62/990,232, filed on Mar. 16, 2020.
(60) Provisional application No. 62/994,734, filed on Mar. 25, 2020.
(60) Provisional application No. 63/015,096, filed on Apr. 24, 2020.

(51) Int. Cl. C07K 16/28

(52) U.S. Cl. CPC C07K 16/2803; C07K 2317/565; C07K 2317/567; C07K 2319/03

(57) ABSTRACT

Engineered NK cells (NK-CAR cells) for targeted immunotherapy of certain cancers are disclosed. Chimeric antigen receptor polypeptides (CAR), and nucleic acids encoding the same, as well as methods of generation of the NK-CAR cells are provided herein. The NK-CAR cells as disclosed herein are capable of, inter alia, binding to TCR receptors, and are useful for treating lymphomas. Also provided are methods of treating cancers using NK-CAR cells.

### CROSS-REFERENCE

This application is a continuation of International Application No. PCT/US2021/022408, filed on Mar. 15, 2021, which claims the benefit of U.S. Provisional Application No. 62/990,232, filed Mar. 16, 2020, U.S. Provisional Application No. 62/994,734, filed Mar. 25, 2020, and U.S. Provisional Application No. 63/015,096, filed Apr. 24, 2020, the entire contents of each of which are hereby incorporated by reference.

### SEQUENCE LISTING

The instant application contains a Sequence Listing which has been submitted electronically in XML format and is hereby incorporated by reference in its entirety. Said XML copy, created on Sep. 14, 2022, is named 53676-705.301SL.xml and is 459,244 bytes in size.

### BACKGROUND

Adoptive immune cell therapy (ACT) is a treatment that involves the administration of immune cells with activity directed against a specific disease related antigen to a subject. Some current compositions and methods comprise the administration of immune cells that express exogenous polypeptides, (e.g., chimeric antigen receptors (CARs) and exogenous T cell receptors (TCRs), wherein the immune cells are modified ex vivo to express the exogenous polypeptides on the surface of the cells, such that when these cells are infused into a subject, the cells are immunologically responsive to a disease causing agent and/or diseased cells. CAR expressing T cells (CAR-T cells) have proven advantageous in clinical trials for B cell malignancies. However, in case of T cell malignancies, CAR T cell therapy faces several challenges including fratricide and T cell aplasia. Thus, a current need exists to develop additional methods of address the deficiencies in cell based therapy at least in case of T cell malignancies.

### SUMMARY

Disclosed herein are, inter alia, methods of adoptive cell therapy for immunological diseases such as infections and cancer. In some embodiments, the adoptive cell therapy focuses on T cell and NK cell therapy, where T cells or NK cells are modified to express a chimeric antigen receptor (CAR). In some embodiments, the cells are genetically modified or engineered ex vivo to express a chimeric antigen receptor. The chimeric antigen receptor is a recombinant fusion protein that comprises an extracellular domain (ECD) a transmembrane domain (TMD) and an intracellular domain (ICD). In some embodiments, the CAR comprises an extracellular domain that can bind to TRBC. In some embodiments, the ECD can specifically bind to TRBC1. In some embodiments, the ECD can specifically bind to TRBC2.

In one aspect, provided herein are methods of generating engineered NK cells, the method comprising: introducing into a population of NK cells, a nucleic acid molecule encoding a chimeric antigen receptor protein (a CAR protein) that comprises an anti-TRBC antigen binding domain, wherein said CAR specifically binds a TRBC protein, to thereby produce a population of NK cells that express said CAR protein on the surface (NK-CAR cells).

In some embodiments, said method further comprises isolating said a plurality of NK cells that express said CAR protein on the surface, to thereby produce said population of NK-CAR cells.

In some embodiments, said NK-CAR cells bind to a cancer cell expressing a TRBC protein on the surface of said cancer cell.

In some embodiments, said TRBC protein is a TRBC1 protein.

In some embodiments, said TRBC protein is a TRBC2 protein.

In some embodiments, said anti-TRBC protein antigen binding domain is an antibody, a ScFv, a bispecific antibody, a diabody, a tribody, a tetrabody, or a functional fragment thereof.

In some embodiments, said anti-TRBC antigen binding domain comprises a heavy chain and a light chain, wherein: said heavy chain comprises an amino acid sequence of at least 95%, 96%, 97%, 98%, 99%, or 100% identity to SEQ ID NO: 176, 177, 178, 179, 180, 181, 182, 183, 184, 185, 196, 197, 199, 200, 201, 203, 205, 207, 209, or 211; and said light chain comprises an amino acid sequence of at least 95%, 96%, 97%, 98%, 99%, or 100% identity to SEQ ID NO: 186, 187, 188, 189, 190, 191, 192, 193, 194, 195, 198, 202, 204, 206, 208, 210, or 212.

In some embodiments, said anti-TRBC antigen binding domain comprises a heavy chain and a light chain, wherein: said heavy chain comprises an amino acid sequence of at least 95%, 96%, 97%, 98%, 99%, or 100% identity to SEQ ID NO: 176, and said light chain comprises an amino acid sequence of at least 95%, 96%, 97%, 98%, 99%, or 100% identity to SEQ ID NO: 186; or, said heavy chain comprises an amino acid sequence of at least 95%, 96%, 97%, 98%, 99%, or 100% identity to any one of SEQ ID NOs: 177, 178, 179, 180, 181, 182, 183, 184 or 185; and said light chain comprises an amino acid sequence of at least 95%, 96%, 97%, 98%, 99%, or 100% identity to any one of SEQ ID NOs: 187, 188, 189, 190, 191, 192, 193, 194 or 195; or, said heavy chain comprises an amino acid sequence of at least 95%, 96%, 97%, 98%, 99%, or 100% identity to any one of SEQ ID NOs: 196, 197 or 199; and said light chain comprises an amino acid sequence of at least 95%, 96%, 97%, 98%, 99%, or 100% identity to SEQ ID NO: 198; or, said heavy chain comprises an amino acid sequence of at least 95%, 96%, 97%, 98%, 99%, or 100% identity to SEQ ID NO: 200 or 201; and said light chain comprises an amino acid sequence of at least 95%, 96%, 97%, 98%, 99%, or 100% identity to SEQ ID NO: 202; or, said heavy chain comprises an amino acid sequence of at least 95%, 96%, 97%, 98%, 99%, or 100% identity to SEQ ID NO: 203, and said light chain comprises an amino acid sequence of at least 95%, 96%, 97%, 98%, 99%, or 100% identity to SEQ ID NO: 204; or, said heavy chain comprises an amino acid sequence of at least 95%, 96%, 97%, 98%, 99%, or 100% identity to SEQ ID NO: 205, and said light chain comprises an amino acid sequence of at least 95%, 96%, 97%, 98%, 99%, or 100% identity to SEQ ID NO: 206; or, said heavy chain comprises an amino acid sequence of at least 95%, 96%, 97%, 98%, 99%, or 100% identity to SEQ ID NO: 207, and said light chain comprises an amino acid sequence of at least 95%, 96%, 97%, 98%, 99%, or 100% identity to SEQ ID NO: 208; or, said heavy chain comprises an amino acid sequence of at least 95%, 96%, 97%, 98%, 99%, or 100% identity to SEQ ID NO: 209, and said light comprises an amino acid sequence of at least 95%, 96%, 97%, 98%, 99%, or 100% identity to SEQ ID NO: 210; or, said heavy chain comprises an amino acid sequence of at least 95%, 96%, 97%, 98%, 99%, or 100% identity to SEQ ID NO: 211, and said light chain comprises an amino acid sequence of at least 95%, 96%, 97%, 98%, 99%, or 100% identity to SEQ ID NO: 212.

In some embodiments, said anti-TRBC antigen binding domain comprises: a heavy chain that comprises a (i) complementarity determinant region (CDR) 1 (CDR1); (ii) a CDR2: (iii) a CDR3; (iv) a framework region 1 (FWR1), (v) a FWR2, (vi) FWR3, and (vii) a FDR4, wherein said CDR1, CDR2, CDR3, FWR1, FWR2, FWR3, and FWR4 comprise sequences at least 95%, 96%, 97%, 98%, 99%, or 100% to those listed in Table 1 or Table 2 and a light chain comprising a (i) complementarity determinant region (CDR)1 (CDR1); (ii) a CDR2: (iii) a CDR3; (iv) a framework region 1 (FWR1), (v) a FWR2, (vi) FWR3, and (vii) a FDR4; wherein said CDR1, CDR2, CDR3, FWR1, FWR2, FWR3, and FWR4 comprise sequences at least 95%, 96%, 97%, 98%, 99%, or 100% to those listed in Table 3.

In some embodiments, said anti-TRBC antigen binding domain comprises a heavy chain and a light chain, wherein: (i) said heavy chain comprises an amino acid sequence of at least 95%, 96%, 97%, 98%, 99%, or 100% identity to SEQ ID NO: 176, 177, 178, 179, 180, 181, 182, 183, 184, or 185; and (ii) said light chain comprises an amino acid sequence of at least 95%, 96%, 97%, 98%, 99%, or 100% identity to SEQ ID NO: 186, 187, 188, 189, 190, 191, 192, 193, 194, or 195.

In some embodiments, said anti-TRBC antigen binding domain comprises a heavy chain and a light chain, wherein: (i) said heavy chain comprises an amino acid sequence of at least 95%, 96%, 97%, 98%, 99%, or 100% identity to SEQ ID NO: 176; and (ii) said light chain comprises an amino acid sequence of at least 95%, 96%, 97%, 98%, 99%, or 100% identity to SEQ ID NO: 186.

In some embodiments, said nucleic acid molecule is a DNA. In some embodiments, said nucleic acid molecule is comprised in a vector. In some embodiments, said nucleic acid molecule is an RNA.

In some embodiments, said NK-CAR cell is cytotoxic to a cell expressing a TRBC protein on the cell surface in vitro. In some embodiments, said NK-CAR cell is cytotoxic to a cell expressing a TRBC protein on the cell surface in vivo. In some embodiments, said NK-CAR cell express TNF-alpha, or interferon gamma, or both, upon contacting a cell expressing said TRBC protein on the cell surface in vitro. In some embodiments, said NK-CAR cell express TNF-alpha, or interferon gamma, or both, upon contacting a cell expressing said TRBC protein on the cell surface in vivo. In some embodiments, said NK-CAR cell expresses at least 1, 2, 3, 4, 5, 6, 7, 8, 9, or 10 cytokines or chemokines selected from the group consisting of IL-1b, IL-6, IL-7, IL-10, IL-12p40, IFN-alpha, MIP-1alpha, MIP-1beta, RANTES and MIG (CXCL9) upon contacting a cell expressing a TRBC protein in vitro. In some embodiments, said NK-CAR cell expresses at least 1, 2, 3, 4, 5, 6, 7, 8, 9, or 10 cytokines or chemokines selected from the group consisting of IL-1b, IL-6, IL-7, IL-10, IL-12p40, IFN-alpha, MIP-1alpha, MIP-1beta, RANTES and MIG (CXCL9) upon contacting a cell expressing a TRBC protein in vivo.

In some embodiments, the method further comprises contacting the population of NK cells with at least one activator. In some embodiments, said at least one activator is selected from the group consisting of an activator protein, a cytokine, an NK cell growth factor, and a blocker of NK cell inhibition, or a nucleic acid encoding any one said activator protein, said cytokine, said NK cell growth factor, or said blocker of NK cell inhibition. In some embodiments, said cytokine is IL-2, IL-12, IL-15, IL-18, or IL-21 or any combination thereof.

In some embodiments, said NK-CAR cell expresses CD56, ICAM-1, CD27, CD48 or CD107a or any combination thereof. In some embodiments, said NK-CAR cell expresses lower levels of CD56 (CD56^{dim}) as determined by a flow cytometry assay, relative to a second population of NK-CAR cells.

In some embodiments, said NK-CAR cell kills a cancer cell expressing said TRBC protein on the cell surface in vitro. In some embodiments, said NK-CAR cell kills a cancer cell expressing said TRBC protein on the cell surface in vivo.

In some embodiments, said NK-CAR cell upon binding a cancer cell expressing said TRBC protein on the cell surface kills said cancer cell. In some embodiments, said cancer cell is a malignant T cell. In some embodiments, said malignant T cell is a cell from a cancer selected from a group consisting of a T cell lymphoma, a non-Hodgkin's lymphoma, an angioimmunoblastic T cell lymphoma, an anaplastic large cell lymphoma, and an acute lymphoblastic leukemia. In some embodiments, said malignant T cell is a CD8+ T cell. In some embodiments, said malignant T cell is a CD4+ T cell. In some embodiments, said NK cells are engineered ex-vivo.

In one aspect, provided herein are methods of treating a T cell malignancy in a subject in need thereof, said method comprising administering to a subject in need thereof an NK cell composition, wherein said NK cell composition comprises a plurality of NK cells that express an exogenous nucleic acid molecule encoding a chimeric receptor (CAR) protein that comprises an anti-TRBC antigen binding domain (NK-CAR cells), wherein said CAR specifically binds to a TRBC protein. In some embodiments, said T cell malignancy is selected from a group consisting of a T cell lymphoma, a non-Hodgkin's lymphoma, an angioimmunoblastic T cell lymphoma, an anaplastic large cell lymphoma, and an acute lymphoblastic leukemia.

In some embodiments, said plurality of NK cells bind to a cancer cell expressing said TRBC protein in vitro. In some embodiments, said plurality of NK cells bind to a cancer cell expressing said TRBC protein in vivo.

In some embodiments, said TRBC protein is a TRBC1 protein. In some embodiments, said TRBC protein is a TRBC2 protein.

In some embodiments, said anti-TRBC antigen binding domain is an antibody, an ScFv, a bispecific antibody, a diabody, a tribody, a tetrabody, or a part thereof.

In some embodiments, said anti-TRBC antigen binding domain comprises a heavy chain and a light chain, wherein: said heavy chain comprises an amino acid sequence of at least 95%, 96%, 97%, 98%, 99%, or 100% identity to SEQ ID NO: 176, 177, 178, 179, 180, 181, 182, 183, 184, 185, 196, 197, 199, 200, 201, 203, 205, 207, 209, or 211; and said light chain comprises an amino acid sequence of at least 95%, 96%, 97%, 98%, 99%, or 100% identity to SEQ ID NO: 186, 187, 188, 189, 190, 191, 192, 193, 194, 195, 198, 202, 204, 206, 208, 210, or 212.

In some embodiments, said anti-TRBC antigen binding domain comprises a heavy chain and a light chain, wherein: said heavy chain comprises an amino acid sequence of at least 95%, 96%, 97%, 98%, 99%, or 100% identity to SEQ ID NO: 176, and said light chain comprises an amino acid sequence of at least 95%, 96%, 97%, 98%, 99%, or 100% identity to SEQ ID NO: 186; or, said heavy chain comprises an amino acid sequence of at least 95%, 96%, 97%, 98%, 99%, or 100% identity to any one of SEQ ID NOs: 177, 178, 179, 180, 181, 182, 183, 184 or 185; and said light chain comprises an amino acid sequence of at least 95%, 96%, 97%, 98%, 99%, or 100% identity to any one of SEQ ID NOs: 187, 188, 189, 190, 191, 192, 193, 194 or 195; said heavy chain comprises an amino acid sequence of at least 95%, 96%, 97%, 98%, 99%, or 100% identity to any one of SEQ ID NOs: 196, 197 or 199; and said light chain comprises an amino acid sequence of at least 95%, 96%, 97%, 98%, 99%, or 100% identity to SEQ ID NO: 198; said heavy chain comprises an amino acid sequence of at least 95%, 96%, 97%, 98%, 99%, or 100% identity to SEQ ID NO: 200 or 201; and said light chain comprises an amino acid sequence of at least 95%, 96%, 97%, 98%, 99%, or 100% identity to SEQ ID NO: 202; said heavy chain comprises an amino acid sequence of at least 95%, 96%, 97%, 98%, 99%, or 100% identity to SEQ ID NO: 203, and said light chain comprises an amino acid sequence of at least 95%, 96%, 97%, 98%, 99%, or 100% identity to SEQ ID NO: 204; or, said heavy chain comprises an amino acid sequence of at least 95%, 96%, 97%, 98%, 99%, or 100% identity to SEQ ID NO: 205, and said light chain comprises an amino acid sequence of at least 95%, 96%, 97%, 98%, 99%, or 100% identity to SEQ ID NO: 206; or, said heavy chain comprises an amino acid sequence of at least 95%, 96%, 97%, 98%, 99%, or 100% identity to SEQ ID NO: 207, and said light chain comprises an amino acid sequence of at least 95%, 96%, 97%, 98%, 99%, or 100% identity to SEQ ID NO: 208; or, said heavy chain comprises an amino acid sequence of at least 95%, 96%, 97%, 98%, 99%, or 100% identity to SEQ ID NO: 209, and said light comprises an amino acid sequence of at least 95%, 96%, 97%, 98%, 99%, or 100% identity to SEQ ID NO: 210; or, said heavy chain comprises an amino acid sequence of at least 95%, 96%, 97%, 98%, 99%, or 100% identity to SEQ ID NO: 211, and said light chain comprises an amino acid sequence of at least 95%, 96%, 97%, 98%, 99%, or 100% identity to SEQ ID NO: 212.

In some embodiments, said TRBC antigen binding domain wherein the TRBC antigen binding domain comprises: a heavy chain comprising a (i) complementarity determinant region (CDR)1 (CDR1); (ii) a CDR2: (iii) a CDR3; (iv) a framework region 1 (FWR1), (v) a FWR2, (vi) FWR3, and (vii) a FDR4, wherein said CDR1, CDR2, CDR3, FWR1, FWR2, FWR3, and FWR4 comprise sequences at least 95%, 96%, 97%, 98%, 99%, or 100% to those listed in Table 1 or Table 2; and a light chain comprising a (i) complementarity determinant region (CDR)1 (CDR1); (ii) a CDR2: (iii) a CDR3; (iv) a framework region 1 (FWR1), (v) a FWR2, (vi) FWR3, and (vii) a FDR4; wherein said CDR1, CDR2, CDR3, FWR1, FWR2, FWR3, and FWR4 comprise sequences at least 95%, 96%, 97%, 98%, 99%, or 100% to those listed in Table 3.

In some embodiments, said anti-TRBC antigen binding domain comprises a heavy chain and a light chain, wherein: (i) said heavy chain comprises an amino acid sequence of at least 95%, 96%, 97%, 98%, 99%, or 100% identity to SEQ ID NO: 176, 177, 178, 179, 180, 181, 182, 183, 184, or 185; and (ii) said light chain comprises an amino acid sequence of at least 95%, 96%, 97%, 98%, 99%, or 100% identity to SEQ ID NO: 186, 187, 188, 189, 190, 191, 192, 193, 194, or 195.

In some embodiments, said anti-TRBC antigen binding domain comprises a heavy chain and a light chain, wherein: (i) said heavy chain comprises an amino acid sequence of at least 95%, 96%, 97%, 98%, 99%, or 100% identity to SEQ ID NO: 176; and (ii) said light chain comprises an amino acid sequence of at least 95%, 96%, 97%, 98%, 99%, or 100% identity to SEQ ID NO: 186.

In some embodiments, said nucleic acid molecule is a DNA. In some embodiments, said nucleic acid molecule is comprised in a vector. In some embodiments, said nucleic acid molecule is an RNA.

In some embodiments, said NK-CAR cells are cytotoxic to a cell expressing said TRBC protein on the cell surface in vitro. In some embodiments, said NK-CAR cells are cytotoxic to a cell expressing said TRBC protein on the cell surface in vivo. In some embodiments, said NK-CAR cells express TNF-alpha, or interferon gamma, or both, upon contacting a cell expressing said TRBC protein on the cell surface in vitro. In some embodiments, said NK-CAR cells express TNF-alpha, or interferon gamma, or both, upon contacting a cell expressing said TRBC protein on the cell surface in vivo. In some embodiments, said NK-CAR cells further expresses at least 1, 2, 3, 4, 5, 6, 7, 8, 9, or 10 cytokines or chemokines selected from the group consisting of IL-1b, IL-6, IL-7, IL-10, IL-12p40, IFN-alpha, MIP-1alpha, MIP-1beta, RANTES and MIG (CXCL9) upon contacting a cell expressing said TRBC protein in vitro. In some embodiments, said NK-CAR cells further expresses at least 1, 2, 3, 4, 5, 6, 7, 8, 9, or 10 cytokines or chemokines selected from the group consisting of IL-1b, IL-6, IL-7, IL-10, IL-12p40, IFN-alpha, MIP-1alpha, MIP-1beta, RANTES and MIG (CXCL9) upon contacting a cell expressing said TRBC protein in vivo. In some embodiments, said NK-CAR cells further expresses CD56, ICAM-1, CD27, CD48 or CD107a or any combination thereof. In some embodiments, said NK-CAR cells further expresses low levels of CD56 (CD56^{dim}) as determined by a flow cytometry assay, relative to a second population of NK-CAR cells.

In some embodiments, said NK-CAR cell kills a cancer cell expressing said TRBC protein in vitro. In some embodiments, said NK-CAR cell kills a cancer cell expressing said TRBC protein in vivo. In some embodiments, said NK cell composition comprises at least 70%, 75%, 80%, 85%, 90%, 95%, 96%, 97%, 98%, or 99% NK-CAR cells (e.g., as measured by flow cytometry).

In some embodiments, NK cell composition comprises 70%, 75%, 80%, 85%, 90%, 95%, 96%, 97%, 98%, or 99% NK-CAR cells that are cytotoxic to a cell expressing said TRBC on the cell surface in vitro, as measured by a standard cytotoxicity assay.

In some embodiments, said NK-CAR cells are allogenic to said subject. In some embodiments, said NK-CAR cells are autologous to said subject.

In some embodiments, said NK-CAR cells express TNF-alpha. In some embodiments, at least 70%, 75%, 80%, 85%, 90%, 95%, or 100% of said NK-CAR cells express TNF-alpha.

In some embodiments, said NK-CAR cells express IFN-γ. In some embodiments, at least 70%, 75%, 80%, 85%, 90%, 95%, or 100% of said NK-CAR cells express IFN-γ.

In one aspect, provided herein are NK-CAR cell compositions produced by a method described herein.

In one aspect, provided herein are pharmaceutical compositions comprising a population of NK-CAR cells produced by the method described herein and a pharmaceutically acceptable excipient.

In one aspect, described herein are chimeric antigen receptor (CAR) polypeptides, wherein said CAR comprises (i) an anti-TRBC antigen binding domain; (ii) a transmembrane domain and (iii) an intracellular domain; wherein said anti-TRBC antigen binding domain specifically binds a TRBC protein (e.g., TRBC1 or TRBC2); wherein the said anti-TRBC antigen binding domain comprises a heavy chain and a light chain.

In some embodiments, said TRBC protein is a TRBC2 protein. In some embodiments, said TRBC protein is a TRBC1 protein.

In some embodiments, (i) said heavy chain comprises an amino acid sequence of at least 95%, 96%, 97%, 98%, 99%, or 100% identity to SEQ ID NO: 176, 177, 178, 179, 180, 181, 182, 183, 184, 185, 196, 197, 199, 200, 201, 203, 205, 207, 209, or 211; and (ii) said light chain comprises an amino acid sequence of at least 95%, 96%, 97%, 98%, 99%, or 100% identity to SEQ ID NO: 186, 187, 188, 189, 190, 191, 192, 193, 194, 195, 198, 202, 204, 206, 208, 210, or 212.

In some embodiments, said heavy chain comprises an amino acid sequence of at least 95%, 96%, 97%, 98%, 99%, or 100% identity to SEQ ID NO: 176, and said light chain comprises an amino acid sequence of at least 95%, 96%, 97%, 98%, 99%, or 100% identity to SEQ ID NO: 186; or, said heavy chain comprises an amino acid sequence of at least 95%, 96%, 97%, 98%, 99%, or 100% identity to any one of SEQ ID NOs: 177, 178, 179, 180, 181, 182, 183, 184 or 185; and said light chain comprises an amino acid sequence of at least 95%, 96%, 97%, 98%, 99%, or 100% identity to any one of SEQ ID NOs: 187, 188, 189, 190, 191, 192, 193, 194 or 195; or, said heavy chain comprises an amino acid sequence of at least 95%, 96%, 97%, 98%, 99%, or 100% identity to any one of SEQ ID NOs: 196, 197 or 199; and said light chain comprises an amino acid sequence of at least 95%, 96%, 97%, 98%, 99%, or 100% identity to SEQ ID NO: 198; or, said heavy chain comprises an amino acid sequence of at least 95%, 96%, 97%, 98%, 99%, or 100% identity to SEQ ID NO: 200 or 201; and said light chain comprises an amino acid sequence of at least 95%, 96%, 97%, 98%, 99%, or 100% identity to SEQ ID NO: 202; or, said heavy chain comprises an amino acid sequence of at least 95%, 96%, 97%, 98%, 99%, or 100% identity to SEQ ID NO: 203, and said light chain comprises an amino acid sequence of at least 95%, 96%, 97%, 98%, 99%, or 100% identity to SEQ ID NO: 204; or, said heavy chain comprises an amino acid sequence of at least 95%, 96%, 97%, 98%, 99%, or 100% identity to SEQ ID NO: 205, and said light chain comprises an amino acid sequence of at least 95%, 96%, 97%, 98%, 99%, or 100% identity to SEQ ID NO: 206; or, said heavy chain comprises an amino acid sequence of at least 95%, 96%, 97%, 98%, 99%, or 100% identity to SEQ ID NO: 207, and said light chain comprises an amino acid sequence of at least 95%, 96%, 97%, 98%, 99%, or 100% identity to SEQ ID NO: 208; or, said heavy chain comprises an amino acid sequence of at least 95%, 96%, 97%, 98%, 99%, or 100% identity to SEQ ID NO: 209, and said light comprises an amino acid sequence of at least 95%, 96%, 97%, 98%, 99%, or 100% identity to SEQ ID NO: 210; or, said heavy chain comprises an amino acid sequence of at least 95%, 96%, 97%, 98%, 99%, or 100% identity to SEQ ID NO: 211, and said light chain comprises an amino acid sequence of at least 95%, 96%, 97%, 98%, 99%, or 100% identity to SEQ ID NO: 212.

In some embodiments, said TRBC antigen binding domain comprises: a heavy chain comprising a (i) complementarity determinant region (CDR)1 (CDR1); (ii) a CDR2: (iii) a CDR3; (iv) a framework region 1 (FWR1), (v) a FWR2, (vi) FWR3, and (vii) a FDR4, wherein said CDR1, CDR2, CDR3, FWR1, FWR2, FWR3, and FWR4 comprise sequences at least 95%, 96%, 97%, 98%, 99%, or 100% to those listed in Table 1 or Table 2; and a light chain comprising a (i) complementarity determinant region (CDR)1 (CDR1); (ii) a CDR2: (iii) a CDR3; (iv) a framework region 1 (FWR1), (v) a FWR2, (vi) FWR3, and (vii) a FDR4; wherein said CDR1, CDR2, CDR3, FWR1, FWR2, FWR3, and FWR4 comprise sequences at least 95%, 96%, 97%, 98%, 99%, or 100% to those listed in Table 3.

In some embodiments, said anti-TRBC antigen binding domain comprises a heavy chain and a light chain, wherein: (i) said heavy chain comprises an amino acid sequence of at least 95%, 96%, 97%, 98%, 99%, or 100% identity to SEQ ID NO: 176, 177, 178, 179, 180, 181, 182, 183, 184, or 185; and (ii) said light chain comprises an amino acid sequence of at least 95%, 96%, 97%, 98%, 99%, or 100% identity to SEQ ID NO: 186, 187, 188, 189, 190, 191, 192, 193, 194, or 195.

In some embodiments, said anti-TRBC antigen binding domain comprises a heavy chain and a light chain, wherein: (i) said heavy chain comprises an amino acid sequence of at least 95%, 96%, 97%, 98%, 99%, or 100% identity to SEQ ID NO: 176; and (ii) said light chain comprises an amino acid sequence of at least 95%, 96%, 97%, 98%, 99%, or 100% identity to SEQ ID NO: 186.

In one aspect, provided herein are nucleic acid molecules encoding a CAR polypeptide described herein.

In one aspect, provided herein are vectors comprising a nucleic acid molecule described herein.

In one aspect, provided herein is a population of lymphocyte cells for example, T cells that express a chimeric antigen receptor (CAR), the CAR having a TRBC antigen binding domain. In one embodiment, the CAR comprises (i) an anti-TRBC antigen binding domain; (ii) a transmembrane domain and (iii) an intracellular domain; wherein said anti-TRBC antigen binding domain specifically binds a TRBC protein (e.g., TRBC1 or TRBC2); wherein the said anti-TRBC antigen binding domain comprises a heavy chain and a light chain.

In some embodiments, said TRBC protein is a TRBC1 protein. In some embodiments, said TRBC protein is a TRBC2 protein.

In some embodiments, (i) said heavy chain comprises an amino acid sequence of at least 95%, 96%, 97%, 98%, 99%, or 100% identity to SEQ ID NO: 176, 177, 178, 179, 180, 181, 182, 183, 184, 185, 196, 197, 199, 200, 201, 203, 205, 207, 209, or 211; and (ii) said light chain comprises an amino acid sequence of at least 95%, 96%, 97%, 98%, 99%, or 100% identity to SEQ ID NO: 186, 187, 188, 189, 190, 191, 192, 193, 194, 195, 198, 202, 204, 206, 208, 210, or 212.

In some embodiments, said heavy chain comprises an amino acid sequence of at least 95%, 96%, 97%, 98%, 99%, or 100% identity to SEQ ID NO: 176, and said light chain comprises an amino acid sequence of at least 95%, 96%, 97%, 98%, 99%, or 100% identity to SEQ ID NO: 186; or, said heavy chain comprises an amino acid sequence of at least 95%, 96%, 97%, 98%, 99%, or 100% identity to any one of SEQ ID NOs: 177, 178, 179, 180, 181, 182, 183, 184 or 185; and said light chain comprises an amino acid sequence of at least 95%, 96%, 97%, 98%, 99%, or 100% identity to any one of SEQ ID NOs: 187, 188, 189, 190, 191, 192, 193, 194 or 195; or, said heavy chain comprises an amino acid sequence of at least 95%, 96%, 97%, 98%, 99%, or 100% identity to any one of SEQ ID NOs: 196, 197 or 199; and said light chain comprises an amino acid sequence of at least 95%, 96%, 97%, 98%, 99%, or 100% identity to SEQ ID NO: 198; or, said heavy chain comprises an amino acid sequence of at least 95%, 96%, 97%, 98%, 99%, or 100% identity to SEQ ID NO: 200 or 201; and said light chain comprises an amino acid sequence of at least 95%, 96%, 97%, 98%, 99%, or 100% identity to SEQ ID NO: 202; or, said heavy chain comprises an amino acid sequence of at least 95%, 96%, 97%, 98%, 99%, or 100% identity to SEQ ID NO: 203, and said light chain comprises an amino acid sequence of at least 95%, 96%, 97%, 98%, 99%, or 100% identity to SEQ ID NO: 204; or, said heavy chain comprises an amino acid sequence of at least 95%, 96%, 97%, 98%, 99%, or 100% identity to SEQ ID NO: 205, and said light chain comprises an amino acid sequence of at least 95%, 96%, 97%, 98%, 99%, or 100% identity to SEQ ID NO: 206; or, said heavy chain comprises an amino acid sequence of at least 95%, 96%, 97%, 98%, 99%, or 100% identity to SEQ ID NO: 207, and said light chain comprises an amino acid sequence of at least 95%, 96%, 97%, 98%, 99%, or 100% identity to SEQ ID NO: 208; or, said heavy chain comprises an amino acid sequence of at least 95%, 96%, 97%, 98%, 99%, or 100% identity to SEQ ID NO: 209, and said light comprises an amino acid sequence of at least 95%, 96%, 97%, 98%, 99%, or 100% identity to SEQ ID NO: 210; or, said heavy chain comprises an amino acid sequence of at least 95%, 96%, 97%, 98%, 99%, or 100% identity to SEQ ID NO: 211, and said light chain comprises an amino acid sequence of at least 95%, 96%, 97%, 98%, 99%, or 100% identity to SEQ ID NO: 212.

In some embodiments, said TRBC antigen binding domain comprises: a heavy chain comprising a (i) complementarity determinant region (CDR)1 (CDR1); (ii) a CDR2: (iii) a CDR3; (iv) a framework region 1 (FWR1), (v) a FWR2, (vi) FWR3, and (vii) a FDR4, wherein said CDR1, CDR2, CDR3, FWR1, FWR2, FWR3, and FWR4 comprise sequences at least 95%, 96%, 97%, 98%, 99%, or 100% to those listed in Table 1 or Table 2; and a light chain comprising a (i) complementarity determinant region (CDR)1 (CDR1); (ii) a CDR2: (iii) a CDR3; (iv) a framework region 1 (FWR1), (v) a FWR2, (vi) FWR3, and (vii) a FDR4; wherein said CDR1, CDR2, CDR3, FWR1, FWR2, FWR3, and FWR4 comprise sequences at least 95%, 96%, 97%, 98%, 99%, or 100% to those listed in Table 3.

In some embodiments, said anti-TRBC antigen binding domain comprises a heavy chain and a light chain, wherein: (i) said heavy chain comprises an amino acid sequence of at least 95%, 96%, 97%, 98%, 99%, or 100% identity to SEQ ID NO: 176, 177, 178, 179, 180, 181, 182, 183, 184, or 185; and (ii) said light chain comprises an amino acid sequence of at least 95%, 96%, 97%, 98%, 99%, or 100% identity to SEQ ID NO: 186, 187, 188, 189, 190, 191, 192, 193, 194, or 195.

In some embodiments, said anti-TRBC antigen binding domain comprises a heavy chain and a light chain, wherein: (i) said heavy chain comprises an amino acid sequence of at least 95%, 96%, 97%, 98%, 99%, or 100% identity to SEQ ID NO: 176; and (ii) said light chain comprises an amino acid sequence of at least 95%, 96%, 97%, 98%, 99%, or 100% identity to SEQ ID NO: 186.

In some embodiments, provided herein are T cell engager molecule, for example, bispecific T cell engages (BiTE). Bispecific T-cell engaging molecules are a class of bispecific antibody-like molecules that are developed for use as anti-cancer drugs. These molecules are soluble, and can specifically engage a T cell and a cancer cell and bring them to proximity. These molecules can direct a T cells' cytotoxic activity against a target cell, such as a cancer cell. In these molecules, one binding domain binds to a T cell via the CD3 receptor, and the other to a target cells such as a tumor cell (via a tumor specific molecule). Since the bispecific molecule binds both the target cell and the T cell, it brings the target cell into proximity with the T cell, so that the T cell can exert its effect, for example, a cytotoxic effect on a cancer cell. The formation of the T cell-bispecific Ab:cancer cell complex induces signaling in the T cell leading to, for example, the release of cytotoxic mediators. Ideally, the agent only induces the desired signaling in the presence of the target cell, leading to selective killing. A BiTE may comprise one antigen binding domain that binds to a TRBC, such as a TRBC1 or TRBC2.

### DETAILED DESCRIPTION

T cells and natural killer (NK) cells possess the ability to kill aberrant cells in the body. These cells are lymphocytes in the same family as T and B cells and originate from a common progenitor, and may be found in the bloodstream and in the lymphatic vessels, placenta, spleen, liver, lungs, tonsils, peripheral ganglia, and bone marrow. NK cells are traditionally characterized as a component of the innate immune system. NK cells are CD3−CD19− cells of the lymphoid lineage. They may express CD16 and CD56 cell surface markers. In human peripheral blood, the NK cell may have a few variety of subtypes based on surface markers: CD56^{bright}, CD16⁻; CD56^{bright}, CD16^{dim}; CD56^{dim}, CD16⁻; CD56^{dim}, CD16^{bright}, which may form the largest NK cell sub-population in peripheral blood; and CD56⁻CD16^{bright}.

NK cells generally respond quickly to a wide variety of pathological challenges. NK cells kill virally infected cells, and detect and control early signs of cancer. Unlike cytotoxic T cells, NK cells do not require priming by antigen presenting cells. These cells respond directly to infected or neoplastic cells through engagement of a multitude of germline-encoded receptors by ligands on target cells. Beside their ability to kill aberrant cells, NK cells are also critical components of the innate immune response by virtue of their capacity to produce a variety of cytokines and chemokines. NK cells secrete cytokines such as IFNγ and TNFα, which act on other immune cells like Macrophage and Dendritic cells to enhance the immune response. While on patrol NK cells constantly contact other cells. Whether or not the NK cell kills these cells depends on a balance of signals from activating receptors and inhibitory receptors on the NK cell surface. Activating receptors recognize molecules that are expressed on the surface of cancer cells and infected cells, and ‘switch on’ the NK cell. Inhibitory receptors act as a check on NK cell killing. Most normal healthy cells express MHC I receptors which mark these cells as ‘self’. Inhibitory receptors on the surface of the NK cell recognize cognate MHC I, and this ‘switches off’ the NK cell, preventing it from killing. Cancer cells and infected cells often lose their MHC I, leaving them vulnerable to NK cell killing. Once the decision is made to kill, the NK cell releases cytotoxic granules containing perforin and granzymes, which leads to lysis of the target cell. The innate cytotoxic nature of NK cells can be further utilized to specifically target cancer cells and destroy cancer cells, especially for cancers in which adoptive T cell therapy is inefficient, such as in case of T cell malignancies.

CAR T cells expressing a CAR having an anti-TRBC binding domain as described herein is also disclosed herein. Recombinant nucleic acid molecule expressing the CAR is expressed in a T cell population to generate CAR T cells, as described herein. In some embodiments, these CAR T cells have the advantage of being generated ex vivo, expanded and can attack cancer cells to generate high cytotoxicity, as well as, through memory T cell activation provides a long term protective effect.

T cell malignancies encompass a heterogeneous group of diseases, each reflecting a clonal evolution of dysfunctional T cells at various stages of development. T cell acute lymphoblastic leukemia (T-ALL) accounts for 15% and 25% of childhood and adult ALL cases respectively, and is the most common form of T cell cancer seen in children. T-lymphoblastic lymphoma (T-LLy) is a non-Hodgkin lymphoma with similar biology to T-ALL. Adult T cell leukemia/lymphoma (ATLL) is an extremely aggressive form of blood cancer driven by the human T cell lymphocytic virus type 1 (HTLV1). Other rare forms of T cell leukemia include T cell large granular lymphocytic leukemia (T-LGL) and T prolymphocytic leukemia (T-PLL). T cell lymphomas are broadly divided into two categories, cutaneous T cell lymphoma (CTCL) and peripheral T cell lymphoma (PTCL). Mycosis fungoides (MF) and Sezary syndrome (SS) represent the two most common subtypes of CTCL, accounting for the majority of cases. PTCL can be classified into several different subtypes, among which include anaplastic large cell lymphoma (ALCL), angioimmunoblastic T cell lymphoma (AITL), extra nodal natural killer (NK)-T cell lymphoma (ENKTL), enteropathy-associated T cell lymphoma (EATL), hepatosplenic T cell lymphoma (HSTCL), and PTCL-not otherwise specified (PTCL-NOS) which is the most common of the group.

Provided herein are chimeric antigen receptor proteins (CAR proteins) that can be expressed in an NK cell or T cell to generate a NK-CAR cell or CAR T cell, respectively. CARs are designed to specifically target a cell surface antigen molecule, such as a cell surface molecule on a particular cancer cell. In some embodiments, CARs comprise an extracellular domain, that is a target antigen binding domain. In some embodiments, a target antigen binding domain is an antibody or a functional fragment thereof. In some embodiments it is a single chain variable fragment (scFv) which serves as the antigen recognition domain. In some embodiments the scFv is derived from a monoclonal antibody. In some embodiments, the antigen binding domain is a bispecific antibody, or a functional fragment thereof. In some embodiments, the antigen binding domain is a diabody, or a functional fragment thereof. In some embodiments, the antigen binding domain is a tribody, or a functional fragment thereof. In some embodiments, the antigen binding domain is a tetrabody, or a functional fragment thereof. In some embodiments, CARs comprise an intracellular signaling domain from the natural T cell receptor (TCR), CD3ζ, linked via a transmembrane domain to a the extracellular antigen recognition domain. In some embodiments, the transmembrane domain is derived from transmembrane protein. A transmembrane domain may be any protein structure which is thermodynamically stable in a membrane. This is typically an alpha helix comprising of several hydrophobic residues. The transmembrane domain of any transmembrane protein can be used to supply the transmembrane portion of the invention. In some embodiments, the transmembrane domain is derived from a receptor, such as, for example, a CD3 or a CD28 molecule. The intracellular cytoplasmic domain (ICD) is the signal-transmission portion of the CAR. It may be part of or associate with the intracellular domain of the TCR. After antigen recognition, receptors cluster, native CD45 and CD148 are excluded from the synapse and a signal is transmitted to the cell. The most commonly used ICD component is that of CD3-zeta which contains 3 ITAMs. This transmits an activation signal to the NK cell after antigen is bound. CD3-zeta may not provide a fully competent activation signal and additional co-stimulatory signaling may be needed.

### Definitions

Unless defined otherwise, all technical and scientific terms used herein have the same meaning as commonly understood by one of ordinary skill in the art.

Ranges: throughout this disclosure, various aspects can be presented in a range format. It should be understood that the description in range format is merely for convenience and brevity and should not be construed as an inflexible limitation on the scope. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

The term “a” and “an” refers to one or to more than one (i.e., to at least one) of the grammatical object of the article. By way of example, “an element” means one element or more than one element.

As used herein, “about” can mean plus or minus less than 1 or 1, 2, 3, 4, 5, 6, 7, 8, 9, 10, 11, 12, 13, 14, 15, 16, 17, 18, 19, 20, 25, 30, or greater than 30 percent, depending upon the situation and known or knowable by one skilled in the art.

Various aspects are described in further detail below. Definitions are set out below and throughout the specification.

The terms “T cell receptor beta constant,” “T cell receptor β constant,” “TRBCB,” “TRBCβ,” are used interchangeably herein and refer to the constant region of the T cell receptor beta chain. There are two T cell receptor β-chain constant domains: TRBC1 and TRBC2.

As used herein, the term “molecule” as used in, e.g., antibody molecule, cytokine molecule, receptor molecule, includes full-length, naturally-occurring molecules, as well as variants, e.g., functional variants (e.g., truncations, fragments, mutated (e.g., substantially similar sequences) or derivatized form thereof), so long as at least one function and/or activity of the unmodified (e.g., naturally-occurring) molecule remains.

The terms “antibody,” and “antibody molecule” as used herein refer to a protein, e.g., an immunoglobulin chain or fragment thereof, comprising at least one immunoglobulin variable domain sequence. An antibody molecule encompasses antibodies (e.g., full-length antibodies) and antibody fragments. Antibodies can be polyclonal or monoclonal, multiple or single chain, or intact immunoglobulins, and may be derived from natural sources or from recombinant sources.

Antibodies can be tetramers of immunoglobulin molecules. In an embodiment, an antibody molecule comprises an antigen binding or functional fragment of a full length antibody, or a full length immunoglobulin chain. For example, a full-length antibody is an immunoglobulin (Ig) molecule (e.g., an IgG antibody) that is naturally occurring or formed by normal immunoglobulin gene fragment recombinatorial processes). In embodiments, an antibody molecule refers to an immunologically active, antigen-binding portion of an immunoglobulin molecule, such as an antibody fragment. The term “antibody fragment” refers to at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen. An antigen binding domain can include a nanobody. In some embodiments, the antigen binding domain can be a non-antibody targeting domain. In some embodiments, the antigen binding domain can be a nanobody. An antibody fragment, e.g., functional fragment, is a portion of an antibody, e.g., Fab, Fab′, F(ab′)₂, F(ab)₂, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full length antibodies and antibody fragments, e.g., dAb (domain antibody), single chain, Fab, Fab′, and F(ab′)₂ fragments, and single chain variable fragments (scFvs). Examples of antibody fragments include, but are not limited to, Fab, Fab′, F(ab′)₂, and Fv fragments, scFv antibody fragments, linear antibodies, single domain antibodies such as sdAb (either V_{L} or V_{H}), camelid V_{H}H domains, and antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies, and is incorporated by reference herein).

As used herein, the term “binding domain” or “binding region” or “antibody molecule” (also referred to herein as “anti-target) refers to a protein, e.g., an immunoglobulin chain or fragment thereof, comprising at least one immunoglobulin variable domain sequence. The term “binding domain” or “antibody molecule” encompasses antibodies and antibody fragments.

The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (u) and lambda (k) light chains refer to the two major antibody light chain isotypes.

The term “recombinant antibody” refers to an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}.

The term “complementarity determining region” or “CDR,” as used herein, refers to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3).

The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme), or a combination thereof. Under the Kabat numbering scheme, in some embodiments, the CDR amino acid residues in the heavy chain variable domain (V_{H}) are numbered 31-35 (HCDR1), 50-65 (HCDR2), and 95-102 (HCDR3); and the CDR amino acid residues in the light chain variable domain (V_{L}) are numbered 24-34 (LCDR1), 50-56 (LCDR2), and 89-97 (LCDR3). Under the Chothia numbering scheme, in some embodiments, the CDR amino acids in the V_{H} are numbered 26-32 (HCDR1), 52-56 (HCDR2), and 95-102 (HCDR3); and the CDR amino acid residues in the V_{L} are numbered 26-32 (LCDR1), 50-52 (LCDR2), and 91-96 (LCDR3). In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HCDR1), 50-65 (HCDR2), and 95-102 (HCDR3) in a V_{H}, e.g., a mammalian V_{H}, e.g., a human V_{H}; and amino acid residues 24-34 (LCDR1), 50-56 (LCDR2), and 89-97 (LCDR3) in a V_{L}, e.g., a mammalian V_{L}, e.g., a human V_{L}.

The portion of the CAR composition comprising an antibody or antibody fragment thereof may exist in a variety of forms where the antigen binding domain is expressed as part of a contiguous polypeptide chain including, for example, a single domain antibody fragment (sdAb), a single chain antibody (scFv) and a humanized or human antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In one aspect, the antigen binding domain of a CAR composition comprises an antibody fragment. In a further aspect, the CAR comprises an antibody fragment that comprises a scFv.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

In embodiments, an antibody molecule is monospecific, e.g., it comprises binding specificity for a single epitope. In some embodiments, an antibody molecule is multispecific, e.g., it comprises a plurality of immunoglobulin variable domain sequences, where a first immunoglobulin variable domain sequence has binding specificity for a first epitope and a second immunoglobulin variable domain sequence has binding specificity for a second epitope. In some embodiments, an antibody molecule is a bispecific antibody molecule. “Bispecific antibody molecule” as used herein refers to an antibody molecule that has specificity for more than one (e.g., two, three, four, or more) epitope and/or antigen.

“Antigen” (Ag) as used herein refers to a molecule that can provoke an immune response, e.g., involving activation of certain immune cells and/or antibody generation. Any macromolecule, including almost all proteins or peptides, can be an antigen. Antigens can also be derived from genomic recombinant or DNA. For example, any DNA comprising a nucleotide sequence or a partial nucleotide sequence that encodes a protein capable of eliciting an immune response encodes an “antigen.” In embodiments, an antigen does not need to be encoded solely by a full length nucleotide sequence of a gene, nor does an antigen need to be encoded by a gene at all. In embodiments, an antigen can be synthesized or can be derived from a biological sample, e.g., a tissue sample, a tumor sample, a cell, or a fluid with other biological components. As used, herein a “tumor antigen” or interchangeably, a “cancer antigen” includes any molecule present on, or associated with, a cancer, e.g., a cancer cell or a tumor microenvironment that can provoke an immune response. As used, herein an “immune cell antigen” includes any molecule present on, or associated with, an immune cell that can provoke an immune response.

The term “cancer associated antigen” or “tumor antigen” or “tumor associated antigen” interchangeably refers to a molecule (typically a protein, carbohydrate or lipid) that is expressed on the surface of a cancer cell, either entirely or as a fragment (e.g., MHC/peptide), and which is useful for the preferential targeting of a pharmacological agent to the cancer cell. In some embodiments, a tumor antigen is a marker expressed by both normal cells and cancer cells. In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (e.g., MHC/peptide), and not synthesized or expressed on the surface of a normal cell. In some embodiments, the CAR includes CARs comprising an antigen binding domain (e.g., antibody or antibody fragment) that binds to an MHC presented peptide. Normally, peptides derived from endogenous proteins fill the pockets of Major histocompatibility complex (MHC) class I molecules, and are recognized by T cell receptors (TCRs) on CD8⁺ T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described (see, e.g., Sastry et al., J. Virol. 2011 85(5):1935-1942; Sergeeva et al., Blood, 2011 117(16):4262-4272; Verma et al., J Immunol 2010 184(4):2156-2165; Willemsen et al., Gene Ther 2001 8(21):1601-1608; Dao et al., Sci Transl Med 2013 5(176):176ra33; Tassev et al., Cancer Gene Ther 2012 19(2):84-100). For example, TCR-like antibody can be identified from screening a library, such as a human scFv phage displayed library.

The “antigen-binding portion” of an antibody molecule refers to the part of an antibody molecule, e.g., an immunoglobulin (Ig) molecule, that participates in antigen binding. In embodiments, the antigen binding site is formed by amino acid residues of the variable (V) regions of the heavy (H) and light (L) chains. Three highly divergent stretches within the variable regions of the heavy and light chains, referred to as hypervariable regions, are disposed between more conserved flanking stretches called “framework regions,” (FRs). FRs are amino acid sequences that are naturally found between, and adjacent to, hypervariable regions in immunoglobulins. In embodiments, in an antibody molecule, the three hypervariable regions of a light chain and the three hypervariable regions of a heavy chain are disposed relative to each other in three dimensional space to form an antigen-binding surface, which is complementary to the three-dimensional surface of a bound antigen. The three hypervariable regions of each of the heavy and light chains are referred to as “complementarity-determining regions,” or “CDRs.” The framework region and CDRs have been defined and described, e.g., in Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917. Each variable chain (e.g., variable heavy chain and variable light chain) is typically made up of three CDRs and four FRs, arranged from amino-terminus to carboxy-terminus in the amino acid order: FR1, CDR1, FR2, CDR2, FR3, CDR3, and FR4.

“Cancer” and “tumor” as used interchangeably herein encompass all types of oncogenic processes and/or cancerous growths. In embodiments, cancer includes primary tumors as well as metastatic tissues or malignantly transformed cells, tissues, or organs. In embodiments, cancer encompasses all histopathologies and stages, e.g., stages of invasiveness/severity, of a cancer. In embodiments, cancer includes relapsed and/or resistant cancer. For example, both terms encompass solid and liquid tumors. As used herein, the term “cancer” or “tumor” includes premalignant, as well as malignant cancers and tumors.

As used herein, an “immune cell” refers to any of various cells that function in the immune system, e.g., to protect against agents of infection and foreign matter. In embodiments, this term includes leukocytes, e.g., neutrophils, eosinophils, basophils, lymphocytes, and monocytes. Innate leukocytes include phagocytes (e.g., macrophages, neutrophils, and dendritic cells), mast cells, eosinophils, basophils, and natural killer cells. Innate leukocytes identify and eliminate pathogens, either by attacking larger pathogens through contact or by engulfing and then killing microorganisms, and are mediators in the activation of an adaptive immune response. The cells of the adaptive immune system are special types of leukocytes, called lymphocytes. B cells and T cells are important types of lymphocytes and are derived from hematopoietic stem cells in the bone marrow. B cells are involved in the humoral immune response, whereas T cells are involved in cell-mediated immune response. The term “immune cell” includes immune effector cells.

“Immune effector cell,” as that term is used herein, refers to a cell that is involved in an immune response, e.g., in the promotion of an immune effector response. Examples of immune effector cells include, but are not limited to, T cells, e.g., alpha/beta T cells and gamma/delta T cells, B cells, natural killer (NK) cells, natural killer T (NK T) cells, and mast cells.

The term “effector function” or “effector response” refers to a specialized function of a cell. Effector function of a T cell, for example, may be cytolytic activity or helper activity including the secretion of cytokines.

The compositions and methods encompass polypeptides and nucleic acids having the sequences specified, or sequences substantially identical or similar thereto, e.g., sequences at least 80%, 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 80%, 85%, 90%. 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 80%, 85%, 90%, 91%, 92%, 93%, 94%, 95%, 96%, 97%, 98% or 99% identity to a reference sequence, e.g., a sequence provided herein.

The term “variant” refers to a polypeptide that has a substantially identical amino acid sequence to a reference amino acid sequence, or is encoded by a substantially identical nucleotide sequence. In some embodiments, the variant is a functional variant.

The term “functional variant” refers to a polypeptide that has a substantially identical amino acid sequence to a reference amino acid sequence, or is encoded by a substantially identical nucleotide sequence, and is capable of having one or more activities of the reference amino acid sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid molecule. BLAST protein searches can be performed with the XBLAST program, score=50, wordlength=3 to obtain amino acid sequences homologous to protein molecules. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used.

It is understood that the molecules may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof, amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The terms “polypeptide”, “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably herein refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement. The term “nucleic acid” includes a gene, cDNA or an mRNA. Unless specifically limited, the term encompasses nucleic acids containing analogues or derivatives of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the coexisting materials in the natural system, is isolated. Such polynucleotides could be part of a vector and/or such polynucleotides or polypeptides could be part of a composition, and still be isolated in that such vector or composition is not part of the environment in which it is found in nature.

The term “chimeric antigen receptor” or alternatively a “CAR” refers to a recombinant polypeptide construct comprising at least an extracellular antigen binding domain, a transmembrane domain and a cytoplasmic signaling domain (also referred to herein as “an intracellular signaling domain”) comprising a functional signaling domain derived from a stimulatory molecule as defined below. In some embodiments, the domains in the CAR polypeptide construct are in the same polypeptide chain, e.g., comprise a chimeric fusion protein. In some embodiments, the domains in the CAR polypeptide construct are not contiguous with each other, e.g., are in different polypeptide chains. In one aspect, the stimulatory molecule of the CAR is the zeta chain associated with the T cell receptor complex. In one aspect, the cytoplasmic signaling domain comprises a primary signaling domain (e.g., a primary signaling domain of CD3-zeta). In one aspect, the cytoplasmic signaling domain further comprises one or more functional signaling domains derived from at least one costimulatory molecule as defined below. In one aspect, the costimulatory molecule is chosen from 4-1BB (i.e., CD137), CD27, ICOS, and/or CD28. In one aspect, the CAR comprises a chimeric fusion protein comprising an extracellular antigen recognition domain, a transmembrane domain and an intracellular signaling domain comprising a functional signaling domain derived from a stimulatory molecule. In one aspect, the CAR comprises a chimeric fusion protein comprising an extracellular antigen recognition domain, a transmembrane domain and an intracellular signaling domain comprising a functional signaling domain derived from a co-stimulatory molecule and a functional signaling domain derived from a stimulatory molecule. In one aspect, the CAR comprises a chimeric fusion protein comprising an extracellular antigen recognition domain, a transmembrane domain and an intracellular signaling domain comprising two functional signaling domains derived from one or more co-stimulatory molecule(s) and a functional signaling domain derived from a stimulatory molecule. In one aspect, the CAR comprises a chimeric fusion protein comprising an extracellular antigen recognition domain, a transmembrane domain and an intracellular signaling domain comprising at least two functional signaling domains derived from one or more co-stimulatory molecule(s) and a functional signaling domain derived from a stimulatory molecule. In one aspect the CAR comprises an optional leader sequence at the amino-terminus (N-ter) of the CAR fusion protein. In one aspect, the CAR further comprises a leader sequence at the N-terminus of the extracellular antigen recognition domain, wherein the leader sequence is optionally cleaved from the antigen recognition domain (e.g., a scFv) during cellular processing and localization of the CAR to the cellular membrane.

The term “signaling domain” refers to the functional portion of a protein which acts by transmitting information within the cell to regulate cellular activity via defined signaling pathways by generating second messengers or functioning as effectors by responding to such messengers.

The term “anti-tumor effect” refers to a biological effect which can be manifested by various means, including but not limited to, e.g., a decrease in tumor volume, a decrease in the number of tumor cells, a decrease in the number of metastases, an increase in life expectancy, decrease in tumor cell proliferation, decrease in tumor cell survival, or amelioration of various physiological symptoms associated with the cancerous condition. An “anti-tumor effect” can also be manifested by the ability of the peptides, polynucleotides, cells and antibodies in prevention of the occurrence of tumor in the first place.

The term “anti-cancer effect” or “anti-tumor effect” refers to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anti-cancer effect” can also be manifested by the ability of the peptides, polynucleotides, cells and antibodies in prevention of the occurrence of cancer in the first place.

The term “autologous” refers to any material derived from the same individual to whom it is later to be re-introduced into the individual.

The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “xenogeneic” refers to a graft derived from an animal of a different species.

The term “apheresis” as used herein refers to the art-recognized extracorporeal process by which the blood of a donor or patient is removed from the donor or patient and passed through an apparatus that separates out selected particular constituent(s) and returns the remainder to the circulation of the donor or patient, e.g., by re-transfusion. Thus, in the context of “an apheresis sample” refers to a sample obtained using apheresis.

The term “combination” refers to either a fixed combination in one dosage unit form, or a combined administration where a compound and a combination partner (e.g. another drug as explained below, also referred to as “therapeutic agent” or “co-agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g. a compound and a combination partner, are both administered to a patient simultaneously in the form of a single entity or dosage. The term “non-fixed combination” means that the active ingredients, e.g. a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients.

“Derived from” as that term is used herein, indicates a relationship between a first and a second molecule. It generally refers to structural similarity between the first molecule and a second molecule and does not connote or include a process or source limitation on a first molecule that is derived from a second molecule. For example, in the case of an intracellular signaling domain that is derived from a CD3zeta molecule, the intracellular signaling domain retains sufficient CD3zeta structure such that is has the required function, namely, the ability to generate a signal under the appropriate conditions. It does not connote or include a limitation to a particular process of producing the intracellular signaling domain, e.g., it does not mean that, to provide the intracellular signaling domain, one must start with a CD3zeta sequence and delete unwanted sequence, or impose mutations, to arrive at the intracellular signaling domain.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment by standard techniques known in the art, such as site-directed mutagenesis and PCR-mediated mutagenesis. Conservative substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a CAR can be replaced with other amino acid residues from the same side chain family and the altered CAR can be tested using the functional assays described herein.

The term “stimulation,” refers to a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-β, and/or reorganization of cytoskeletal structures, and the like.

The term “stimulatory molecule,” refers to a molecule expressed by a T cell that provides the primary cytoplasmic signaling sequence(s) that regulate primary activation of the TCR complex in a stimulatory way for at least some aspect of the T cell signaling pathway. In one aspect, the primary signal is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immune receptor tyrosine-based activation motif or ITAM. Examples of an ITAM containing primary cytoplasmic signaling sequence that is of particular use includes, but is not limited to, those derived from TCR zeta, FcR gamma, FcR beta, CD3 gamma, CD3 delta, CD3 epsilon, CD5, CD22, CD79a, CD79b, CD278 (also known as “ICOS”), FcεRI, CD66d, DAP10 and DAP12. In a specific CAR, the intracellular signaling domain in any one or more CARS comprises an intracellular signaling sequence, e.g., a primary signaling sequence of CD3-zeta.

The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T-cells may recognize these complexes using their T-cell receptors (TCRs). APCs process antigens and present them to T-cells.

An “intracellular signaling domain,” as the term is used herein, refers to an intracellular portion of a molecule. The intracellular signaling domain can generate a signal that promotes an immune effector function of the CAR containing cell, e.g., a CART cell or CAR-expressing NK cell. Examples of immune effector function, e.g., in a CART cell or CAR-expressing NK cell, include cytolytic activity and helper activity, including the secretion of cytokines. In embodiments, the intracellular signal domain transduces the effector function signal and directs the cell to perform a specialized function. While the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain.

Exemplary costimulatory intracellular signaling domains include those derived from molecules responsible for costimulatory signals, or antigen independent stimulation. For example, in the case of a CAR-expressing immune effector cell, e.g., CART cell or CAR-expressing NK cell, a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, FcR gamma, FcR beta, CD3 gamma, CD3 delta, CD3 epsilon, CDS, CD22, CD79a, CD79b, CD278 (“ICOS”), FcεRI, CD66d, DAP10, and DAP12.

The term “zeta” or alternatively “zeta chain”, “CD3-zeta” or “TCR-zeta” is defined as the protein provided as GenBank Acc. No. BAG36664.1, or the equivalent residues from a non-human species, e.g., mouse, rodent, monkey, ape and the like, and a “zeta stimulatory domain” or alternatively a “CD3-zeta stimulatory domain” or a “TCR-zeta stimulatory domain” is defined as the amino acid residues from the cytoplasmic domain of the zeta chain that are sufficient to functionally transmit an initial signal necessary for T cell activation. In one aspect the cytoplasmic domain of zeta comprises residues 52 through 164 of GenBank Acc. No. BAG36664.1 or the equivalent residues from a non-human species, e.g., mouse, rodent, monkey, ape and the like, that are functional orthologs thereof.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to an a MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD1 Ic, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

A costimulatory intracellular signaling domain refers to the intracellular portion of a costimulatory molecule. The intracellular signaling domain can comprise the entire intracellular portion, or the entire native intracellular signaling domain, of the molecule from which it is derived, or a functional fragment thereof.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

The term “effective amount” or “therapeutically effective amount” are used interchangeably herein, and refer to an amount of a compound, formulation, material, or composition, as described herein effective to achieve a particular biological result.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence driven by a promoter.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “vector” as used herein refers to any vehicle that can be used to deliver and/or express a nucleic acid molecule. It can be a transfer vector or an expression vector as described herein.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR@ gene delivery technology from Oxford BioMedica, the LENTIMAX^{T}M vector system from Lentigen and the like. Nonclinical types of lentiviral vectors are also available and would be known to one skilled in the art.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody or antibody fragment, where the whole molecule is of human origin or consists of an amino acid sequence identical to a human form of the antibody or immunoglobulin.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context, the following abbreviations for the commonly occurring nucleic acid bases are used. “A” refers to adenosine, “C” refers to cytosine, “G” refers to guanosine, “T” refers to thymidine, and “U” refers to uridine.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “parenteral” administration of an immunogenic composition includes, e.g., subcutaneous (s.c.), intravenous (i.v.), intramuscular (i.m.), or intrasternal injection, intratumoral, or infusion techniques.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive” promoter refers to a nucleotide sequence which, when operably linked with a polynucleotide which encodes or specifies a gene product, causes the gene product to be produced in a cell under most or all physiological conditions of the cell.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific” promoter refers to a nucleotide sequence which, when operably linked with a polynucleotide encodes or specified by a gene, causes the gene product to be produced in a cell substantially only if the cell is a cell of the tissue type corresponding to the promoter.

The term “flexible polypeptide linker” as used in the context of a scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Ser)n, where n is a positive integer equal to or greater than 1 (SEQ ID NO: 344). For example, n=1, n=2, n=3. n=4, n=5 and n=6, n=7, n=8, n=9 and n=10 In one embodiment, the flexible polypeptide linkers include, but are not limited to, (Gly₄Ser)₄ (SEQ ID NO: 345) or (Gly₄Ser)₃ (SEQ ID NO: 346). In another embodiment, the linkers include multiple repeats of (Gly₂Ser), (GlySer) or (Gly₃Ser) (SEQ ID NO: 347). Also included within the scope are linkers described in WO2012/138475, incorporated herein by reference).

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

As used herein, the terms “treat,” “treatment,” and “treating” refer to the reduction or amelioration of the progression, severity and/or duration of a proliferative disorder, or the amelioration of one or more symptoms (preferably, one or more discernible symptoms) of a proliferative disorder resulting from the administration of one or more therapies (e.g., one or more therapeutic agents such as a CAR). In specific embodiments, the terms “treat,” “treatment,” and “treating” refer to the amelioration of at least one measurable physical parameter of a proliferative disorder, such as growth of a tumor, not necessarily discernible by the patient. In other embodiments the terms “treat”, “treatment” and “treating”-refer to the inhibition of the progression of a proliferative disorder, either physically by, e.g., stabilization of a discernible symptom, physiologically by, e.g., stabilization of a physical parameter, or both. In other embodiments the terms “treat,” “treatment,” and “treating” refer to the reduction or stabilization of tumor size or cancerous cell count.

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The terms “subject” and “patient” are used interchangeably herein and are intended to include living organisms in which an immune response can be elicited (e.g., mammals, human).

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “specifically binds,” refers to an antibody, or a ligand, which recognizes and binds with a cognate binding partner (e.g., a stimulatory and/or costimulatory molecule present on a T cell) protein present in a sample, but which antibody or ligand does not substantially recognize or bind other molecules in the sample.

### Overview

Provided herein are chimeric antigen receptor proteins (CAR proteins) that comprise an anti-T cell receptor beta chain constant region (TRBC) antigen binding domain that can be expressed in NK cells or T cells. T cell receptors (TCRs) are receptors found on the surface of lymphocytes, specifically on T lymphocytes (T cells). TCRs are responsible for recognizing antigen fragments presented by major histocompatibility complex (MHC) molecules on other immune cells (e.g., B cells) by signaling through associated CD3 and activating the T cell. The majority of TCRs in humans are heterodimers comprising an alpha chain and a beta chain. Both alpha and beta chains of TCR comprise variable and constant regions. The variable regions of the alpha and beta chain are encoded by distinct DNA elements (V, D, and J elements for beta chain; V and J elements for the alpha chain). Recombination between these elements produces in large part the variation in antigen binding specificity of TCRs. The TCR beta chain constant region is selected from two different domains, beta constant domain 1 and beta constant domain 2.

In some embodiments, the CAR molecules of the present application comprise an antigen binding domain that binds to a tumor antigen on a lymphoma cell (e.g., a T cell), e.g., a T cell receptor comprising TRBC1, TRBC1, a T cell receptor comprising TRBC2, or TRBC2. In some embodiments, the CAR molecules or antibody molecules as described herein comprise an antigen binding domain that selectively targets lymphocytes expressing a T cell receptor comprising TRBC1, TRBC1, a T cell receptor comprising TRBC2, or TRBC2. In some embodiments, the lymphoma cell or lymphocyte may not be a T cell. In some embodiments, the lymphoma cell or lymphocyte is a B cell.

### TRBC Binding Domains

The T-cell receptor is formed from six different protein chains which must assemble in the endoplasmic reticulum to be expressed on the cell surface. Of the six different protein chains four proteins of the CD3 complex (CD3ζ, CD3γ, CD3ε and CD3δ) sheath the T-cell Receptor (TCR). This TCR imbues the complex with specificity for a particular antigen and is composed of two chains: TCR alpha chain (TCRA, or TRA) and TCR beta chain (TCRB or TRB). Each TCR chain has a variable component distal to the membrane and a constant component proximal to the membrane.

In some embodiments, a CAR described herein comprises an antigen binding domain that specifically binds a TRBC protein. In some embodiments, said TRBC protein is TRBC1. In some embodiments, said TRBC2 protein.

In some embodiments, the antigen binding domain comprises an antibody or functional fragment or variant thereof. In some embodiments, the antigen binding domain is a full length antibody, a Fab, or a scFv. In some embodiments, the antigen binding domain is a scFv. In some embodiments, the antigen binding domain comprises at least one, two, or three CDRs from Tables 1-5. In some embodiments, the antigen binding domain comprises at least one, two, three, or four FW regions from Tables 1-5. The anti-TCRB protein antigen binding domain may constitute an antigen binding domain that binds to a tumor antigen on a lymphoma cell (e.g., a T cell), e.g., a T cell receptor comprising T cell receptor beta chain constant domain 1 (TRBC1) or a T cell receptor comprising T cell receptor beta chain constant domain 2 (TRBC2). The anti-TCRB protein antigen binding domain can target a cell that expresses on its surface an antigen targeted by the TCRB, such antigen may be expressed on the surface of a cancer cell. Without wishing to be bound by theory, clonally derived T cell lymphomas are positive for either TRBC1 or TRBC2, but not both. In the case of TRBC1+ T cell malignancies, an anti-TRBC1 molecule disclosed herein (e.g., a CAR molecule that binds to TRBC1) may deplete TRBC1+ cells while sparing TRBC2+ non-malignant T cells. Similarly, in the case of TRBC2+ T cell malignancies, an anti-TRBC2 molecule disclosed herein may deplete TRBC2+ cells while sparing TRBC1+ non-malignant T cells.

In some embodiments, the tumor antigen comprises a T cell receptor comprising TRBC1 or TRBC2. Without wising to be bound by theory, in some embodiments, a CAR disclosed herein (e.g., comprising an anti-TRBC1 antigen binding domain or antibody) only activates NK cells in the presence of a TRBC1-expressing cell. Without wising to be bound by theory, in some embodiments, a CAR disclosed herein (e.g., comprising an anti-TRBC2 antigen binding domain or antibody) only activates NK cells in the presence of a TRBC2-expressing cell. In some embodiments, the antigen binding domain preferentially binds to a T cell receptor comprising TRBC1 (e.g., relative to a T cell receptor comprising TRBC2). In some embodiments, the antigen binding domain preferentially binds to a T cell receptor comprising TRBC2 (e.g., relative to a T cell receptor comprising TRBC1). In some embodiments, the CAR molecules include, e.g., are engineered to contain, one or more antigen binding domains that selectively target lymphocytes expressing TRBC1 or TRBC2. In some embodiments, the antigen binding domain selectively targets lymphocytes expressing a T cell receptor comprising TRBC1 or a T cell receptor comprising TRBC2.

In some embodiments, the antigen binding domain (e.g., first antigen binding domain) that binds to a tumor antigen on a lymphoma cell (e.g., a T cell), e.g., a T cell receptor comprising TRBC1, TRBC1, a T cell receptor comprising TRBC2, or TRBC2 comprises any CDR amino acid sequence, framework region (FWR) amino acid sequence, or variable region amino acid sequence Tables 1-5. In some embodiments, the antigen binding domain that binds to a tumor antigen on a lymphoma cell (e.g., a T cell), e.g., a T cell receptor comprising TRBC1, TRBC1, a T cell receptor comprising TRBC2, or TRBC2 comprises heavy and/or light chain amino acid sequences of Table 5. In some embodiments, the antigen binding domain that selectively targets lymphocytes expressing a T cell receptor comprising TRBC1, TRBC1, a T cell receptor comprising TRBC2, or TRBC2 comprises any CDR amino acid sequence, framework region (FWR) amino acid sequence, or variable region amino acid sequence disclosed in Tables 1-6. In some embodiments, the antigen binding domain that selectively targets lymphocytes expressing a T cell receptor comprising TRBC1, TRBC1, a T cell receptor comprising TRBC2, or TRBC2 comprises heavy and/or light chain amino acid sequences of Table 5. An antigen binding domain that binds to a tumor antigen comprising TRBC1 or selectively targets lymphocytes expressing TRBC1 may be said to target TRBC1 (i.e., a TRBC1-targeting antigen binding domain). An antigen binding domain that binds to a tumor antigen comprising TRBC2 or selectively targets lymphocytes expressing TRBC2 may be said to target TRBC2 (i.e., a TRBC2-targeting antigen binding domain).

In some embodiments, provided herein is an antibody molecule that binds to TRBC1, comprising one or more CDRs, framework regions, variable regions, or antigen binding domains disclosed in any of Tables 1-5 and 6, or a sequence having at least 85%, 90%, 95%, or 99% identity thereto.

In some embodiments, the antigen binding domain comprises a VH comprising a heavy chain complementarity determining region 1 (VHCDR1), a VHCDR2, and a VHCDR3, and a VL comprising a light chain complementarity determining region 1 (VLCDR1), a VLCDR2, and a VLCDR3, wherein the VHCDR1, VHCDR2, and VHCDR3 comprise the amino acid sequences of: SEQ ID NOs: 2, 4, 6 respectively. In some embodiments, the antigen binding domain comprises a VH comprising a VHCDR1, VHCDR2, and VHCDR3 that comprise the amino acid sequences of: SEQ ID NOs: 9, 11, 13 respectively. In some embodiments, the antigen binding domain comprises a VH comprising a VHCDR1, VHCDR2, and VHCDR3 that comprise the amino acid sequences of: SEQ ID NOs: 16, 18, 20 respectively. In some embodiments, the antigen binding domain comprises a VH comprising a VHCDR1, VHCDR2, and VHCDR3 that comprise the amino acid sequences of: SEQ ID NOs: 23, 25, 27 respectively. In some embodiments, the antigen binding domain comprises a VH comprising a VHCDR1, VHCDR2, and VHCDR3 that comprise the amino acid sequences of: SEQ ID NOs: 30, 32, 34 respectively.

In some embodiments, a CAR described herein comprising anti-TRBC antigen binding domain that comprises a heavy chain and a light chain, wherein: (a) said heavy chain comprises an amino acid sequence of at least 95%, 96%, 97%, 98%, 99%, or 100% identity to SEQ ID NO: 176, and said light chain comprises an amino acid sequence of at least 95%, 96%, 97%, 98%, 99%, or 100% identity to SEQ ID NO: 186; or, (b) said heavy chain comprises an amino acid sequence of at least 95%, 96%, 97%, 98%, 99%, or 100% identity to any one of SEQ ID NOs: 177, 178, 179, 180, 181, 182, 183, 184 or 185; and said light chain comprises an amino acid sequence of at least 95%, 96%, 97%, 98%, 99%, or 100% identity to any one of SEQ ID NOs: 187, 188, 189, 190, 191, 192, 193, 194 or 195; or (c) said heavy chain comprises an amino acid sequence of at least 95%, 96%, 97%, 98%, 99%, or 100% identity to any one of SEQ ID NOs: 196, 197 or 199; and said light chain comprises an amino acid sequence of at least 95%, 96%, 97%, 98%, 99%, or 100% identity to SEQ ID NO: 198; (d) said heavy chain comprises an amino acid sequence of at least 95%, 96%, 97%, 98%, 99%, or 100% identity to SEQ ID NO: 200 or 201; and said light chain comprises an amino acid sequence of at least 95%, 96%, 97%, 98%, 99%, or 100% identity to SEQ ID NO: 202; (e) said heavy chain comprises an amino acid sequence of at least 95%, 96%, 97%, 98%, 99%, or 100% identity to SEQ ID NO: 203, and said light chain comprises an amino acid sequence of at least 95%, 96%, 97%, 98%, 99%, or 100% identity to SEQ ID NO: 204; or, (f) said heavy chain comprises an amino acid sequence of at least 95%, 96%, 97%, 98%, 99%, or 100% identity to SEQ ID NO: 205, and said light chain comprises an amino acid sequence of at least 95%, 96%, 97%, 98%, 99%, or 100% identity to SEQ ID NO: 206; or, (g) said heavy chain comprises an amino acid sequence of at least 95%, 96%, 97%, 98%, 99%, or 100% identity to SEQ ID NO: 207, and said light chain comprises an amino acid sequence of at least 95%, 96%, 97%, 98%, 99%, or 100% identity to SEQ ID NO: 208; or, (h) said heavy chain comprises an amino acid sequence of at least 95%, 96%, 97%, 98%, 99%, or 100% identity to SEQ ID NO: 209, and said light comprises an amino acid sequence of at least 95%, 96%, 97%, 98%, 99%, or 100% identity to SEQ ID NO: 210; or, (i) said heavy chain comprises an amino acid sequence of at least 95%, 96%, 97%, 98%, 99%, or 100% identity to SEQ ID NO: 211, and said light chain comprises an amino acid sequence of at least 95%, 96%, 97%, 98%, 99%, or 100% identity to SEQ ID NO: 212.

TABLE 1
Exemplary heavy chain CDRs and FWRs of TRBC1-targeting antigen
binding domains derived from JOVI.1

| Ab ID | VHFWR1 | VHCDR1 | VHFWR2 | VHCDR2 | VHFWR3 | VHCDR3 | VHFWR4 |
|---|---|---|---|---|---|---|---|
| mJOVI.1-H | EVRLQQ | FTGYVM | WVKQRP | FINPYND | KATLTSD | GAGYNF | WGQGTT |
| | SGPDLIK | H(SEQ | GQGLEW | DIQSNER | KSSTTAY | DGAYRF | LTVSS |
| | PGASVK | ID NO: 2) | IG (SEQ | FRG | MELSSLT | FDF (SEQ | (SEQ ID |
| | MSCKAS | | ID NO: 3) | (SEQ ID | SEDSAVY | ID NO: 6) | NO: 7) |
| | GYT | | | NO: 4) | YCAR | | |
| | (SEQ ID | | | | (SEQ ID | | |
| | NO: 1) | | | | NO: 5) | | |
| h1JOVI.1-H | QVQLVQ | FTGYVM | WVRQAP | FINPYND | RVTMTS | GAGYNF | WGQGTL |
| | SGAEVK | H(SEQ | GQGLEW | DIQSNER | DKSTTTA | DGAYRF | VTVSS |
| | KPGASV | ID NO: 9) | MG (SEQ | FRG | YMELSSL | FDF (SEQ | (SEQ ID |
| | KVSCKA | | ID NO: | (SEQ ID | RSEDTAV | ID NO: | NO: 14) |
| | SGYT | | 10) | NO: 11) | YYCAR | 13) | |
| | (SEQ ID | | | | (SEQ ID | | |
| | NO: 8) | | | | NO: 12) | | |
| h2JOVI.1-H | QVQLVQ | FTGYVM | WVRQAP | FINPYND | WVTMTS | GAGYNF | WGQGTL |
| | SGAEVK | H(SEQ | GQGLEW | DIQSNER | DKSITTA | DGAYRF | VTVSS |
| | KPGASV | ID NO: | MG (SEQ | FRG | YMELSRL | FDF (SEQ | (SEQ ID |
| | KVSCKA | 16) | ID NO: | (SEQ ID | RSDDTAV | ID NO: | NO: 21) |
| | SGYT | | 17) | NO: 18) | YYCAR | 20) | |
| | (SEQ ID | | | | (SEQ ID | | |
| | NO: 15) | | | | NO: 19) | | |
| h3JOVI.1-H | QVQLVQ | FTGYVM | WVRQAP | FINPYND | RVTITSD | GAGYNF | WGQGTL |
| | SGAEVK | H(SEQ | GQGLEW | DIQSNER | KSTTTAY | DGAYRF | VTVSS |
| | KPGSSV | ID NO: | MG (SEQ | FRG | MELSSLR | FDF (SEQ | (SEQ ID |
| | KVSCKA | 23) | ID NO: | (SEQ ID | SEDTAVY | ID NO: | NO: 28) |
| | SGYT | | 24) | NO: 25) | YCAR | 27) | |
| | (SEQ ID | | | | (SEQ ID | | |
| | NO: 22) | | | | NO: 26) | | |
| h4JOVI.1-H | QVQLVQ | FTGYVM | WVRQAP | FINPYND | RVTITSD | GAGYNF | WGQGTL |
| | SGAEVK | H(SEQ | GQRLEW | DIQSNER | KSATTAY | DGAYRF | VTVSS |
| | KPGASV | ID NO: | MG (SEQ | FRG | MELSSLR | FDF (SEQ | (SEQ ID |
| | KVSCKA | 30) | ID NO: | (SEQ ID | SEDTAVY | ID NO: | NO: 35) |
| | SGYT | | 31) | NO: 32) | YCAR | 34) | |
| | (SEQ ID | | | | (SEQ ID | | |
| | NO: 29) | | | | NO: 33) | | |

TABLE 2
Exemplary heavy chain CDRs and FWRs of TRBC1-targeting antigen binding
domains derived from JOVI.1 (according to the Kabat numbering scheme)

| Ab ID | VHFWR1 | VHCDR1 | VHFWR2 | VHCDR2 | VHFWR3 | VHCDR3 | VHFWR4 |
|---|---|---|---|---|---|---|---|
| mJOVI.1-H | EVRLQQ | GYVMH | WVKQRP | FINPYND | KATLTSD | GAGYNF | WGQGTT |
| | SGPDLIK | (SEQ ID | GQGLEW | DIQSNER | KSSTTAY | DGAYRF | LTVSS |
| | PGASVK | NO: 37) | IG (SEQ | FRG | MELSSLT | FDF (SEQ | (SEQ ID |
| | MSCKAS | | ID NO: | (SEQ ID | SEDSAVY | ID NO: | NO: 42) |
| | GYTFT | | 38) | NO: 39) | YCAR | 41) | |
| | (SEQ ID | | | | (SEQ ID | | |
| | NO: 36) | | | | NO: 40) | | |
| h1JOVI.1-H | QVQLVQ | GYVMH | WVRQAP | FINPYND | RVTMTS | GAGYNF | WGQGTL |
| | SGAEVK | (SEQ ID | GQGLEW | DIQSNER | DKSTTTA | DGAYRF | VTVSS |
| | KPGASV | NO: 44) | MG (SEQ | FRG | YMELSSL | FDF (SEQ | (SEQ ID |
| | KVSCKA | | ID NO: | (SEQ ID | RSEDTAV | ID NO: | NO: 49) |
| | SGYTFT | | 45) | NO: 46) | YYCAR | 48) | |
| | (SEQ ID | | | | (SEQ ID | | |
| | NO: 43) | | | | NO: 47) | | |
| h2JOVI.1-H | QVQLVQ | GYVMH | WVRQAP | FINPYND | WVTMTS | GAGYNF | WGQGTL |
| | SGAEVK | (SEQ ID | GQGLEW | DIQSNER | DKSITTA | DGAYRF | VTVSS |
| | KPGASV | NO: 51) | MG (SEQ | FRG | YMELSRL | FDF (SEQ | (SEQ ID |
| | KVSCKA | | ID NO: | (SEQ ID | RSDDTAV | ID NO: | NO: 56) |
| | SGYTFT | | 52) | NO: 53) | YYCAR | 55) | |
| | (SEQ ID | | | | (SEQ ID | | |
| | NO: 50) | | | | NO: 54) | | |
| h3JOVI.1-H | QVQLVQ | GYVMH | WVRQAP | FINPYND | RVTITSD | GAGYNF | WGQGTL |
| | SGAEVK | (SEQ ID | GQGLEW | DIQSNER | KSTTTAY | DGAYRF | VTVSS |
| | KPGSSV | NO: 58) | MG (SEQ | FRG | MELSSLR | FDF (SEQ | (SEQ ID |
| | KVSCKA | | ID NO: | (SEQ ID | SEDTAVY | ID NO: | NO: 63) |
| | SGYTFT | | 59) | NO: 60) | YCAR | 62) | |
| | (SEQ ID | | | | (SEQ ID | | |
| | NO: 57) | | | | NO: 61) | | |
| h4JOVI.1-H | QVQLVQ | GYVMH | WVRQAP | FINPYND | RVTITSD | GAGYNF | WGQGTL |
| | SGAEVK | (SEQ ID | GQRLEW | DIQSNER | KSATTAY | DGAYRF | VTVSS |
| | KPGASV | NO: 65) | MG (SEQ | FRG | MELSSLR | FDF (SEQ | (SEQ ID |
| | KVSCKA | | ID NO: | (SEQ ID | SEDTAVY | ID NO: | NO: 70) |
| | SGYTFT | | 66) | NO: 67) | YCAR | 69) | |
| | (SEQ ID | | | | (SEQ ID | | |
| | NO: 64) | | | | NO: 68) | | |
| h5JOVI.1-H | QVQLVQ | GYVMH | WVRQAP | FINPYND | RVTITSD | GAGYNF | WGQGTT |
| | SGAEVK | (SEQ ID | GQGLEW | DIQSNER | KSTTTAY | DGAYRF | VTVSS |
| | KPGSSV | NO: 72) | MG (SEQ | FRG | MELSSLR | FDF (SEQ | (SEQ ID |
| | KVSCKA | | ID NO: | (SEQ ID | SEDTAVY | ID NO: | NO: 77) |
| | SGYTFT | | 73) | NO: 74) | YCAR | 76) | |
| | (SEQ ID | | | | (SEQ ID | | |
| | NO: 71) | | | | NO: 75) | | |
| h6JOVI.1-H | QVQLVQ | GYVMH | WVRQAP | FINPYND | RVTMTS | GAGYNF | WGQGTT |
| | SGAEVK | (SEQ ID | GQGLEW | DIQSNER | DKSITTA | DGAYRF | VTVSS |
| | KPGASV | NO: 79) | MG (SEQ | FRG | YMELSRL | FDF (SEQ | (SEQ ID |
| | KVSCKA | | ID NO: | (SEQ ID | RSDDTAV | ID NO: | NO: 84) |
| | SGYTFT | | 80) | NO: 81) | YYCAR | 83) | |
| | (SEQ ID | | | | (SEQ ID | | |
| | NO: 78) | | | | NO: 82) | | |
| H1 | QVQLVQ | GYAIS | WVRQAP | FINPYND | RVTITSD | GAGYNF | WGQGTL |
| germlined- | SGAEVK | (SEQ ID | GQGLEW | DIQSNER | KSTTTAY | DGAYRF | VTVSS |
| VH | KPGSSV | NO: 86) | MG (SEQ | FRG | MELSSLR | FDF (SEQ | (SEQ ID |
| | KVSCKA | | ID NO: | (SEQ ID | SEDTAVY | ID NO: | NO: 91) |
| | SGYTFS | | 87) | NO: 88) | YCAR | 90) | |
| | (SEQ ID | | | | (SEQ ID | | |
| | NO: 85) | | | | NO: 89) | | |
| H2 | QVQLVQ | GYVMH | WVRQAP | FIIPIFGT | RVTITSD | GAGYNF | WGQGTL |
| germlined- | SGAEVK | (SEQ ID | GQGLEW | ANYAQK | KSTTTAY | DGAYRF | VTVSS |
| VH | KPGSSV | NO: 93) | MG (SEQ | FQG | MELSSLR | FDF (SEQ | (SEQ ID |
| | KVSCKA | | ID NO: | (SEQ ID | SEDTAVY | ID NO: | NO: 98) |
| | SGYTFT | | 94) | NO: 95) | YCAR | 97) | |
| | (SEQ ID | | | | (SEQ ID | | |
| | NO: 92) | | | | NO: 96) | | |
| H1/H2 | QVQLVQ | GYAIS | WVRQAP | FIIPIFGT | RVTITSD | GAGYNF | WGQGTL |
| germlined- | SGAEVK | (SEQ ID | GQGLEW | ANYAQK | KSTTTAY | DGAYRF | VTVSS |
| VH | KPGSSV | NO: 100) | MG (SEQ | FQG | MELSSLR | FDF (SEQ | (SEQ ID |
| | KVSCKA | | ID NO: | (SEQ ID | SEDTAVY | ID NO: | NO: 105) |
| | SGYTFS | | 101) | NO: 102) | YCAR | 104) | |
| | (SEQ ID | | | | (SEQ ID | | |
| | NO: 99) | | | | NO: 103) | | |

In some embodiments, the VHCDR2, VHCDR2, and VHCDR3 of the antigen binding domains comprise, according to Kabat numbering sequence, the amino acid sequences of: SEQ ID NOs: 37, 39, 41 respectively; or SEQ ID NOs: 44, 46, 48 respectively; or SEQ ID NOs: 51, 53, 55 respectively; or SEQ ID NOs: 58, 60, 62 respectively; or SEQ ID NOs: 65, 67, 69 respectively; or SEQ ID NOs: 72, 74, 76 respectively; or SEQ ID NOs: 79, 81, 83 respectively; or SEQ ID NOs: 86, 88, 90 respectively; or SEQ ID NOs: 93, 95, 97 respectively or SEQ ID NOs: 100, 102, 104 respectively.

In one aspect, the antigen binding domain comprises a variable light chain domain, VL, comprising a VLCDR1, VLCDR2, and VLCDR3 that comprise the amino acid sequences of: SEQ ID NOs: 107, 109, 111 respectively. In some embodiments, the VLCDR1, VLCDR2 and a VLCDR3 that comprise the amino acid sequences of SEQ ID NOs: 114, 116, 118 respectively; or SEQ ID NOs: 121, 123, 125 respectively; or SEQ ID NOs: 128, 130, 132 respectively; SEQ ID NOs: 135, 137, 139 respectively; or SEQ ID NOs: 142, 144, 146 respectively; SEQ ID NOs: 149, 151, 153 respectively; SEQ ID NOs: 156, 158, 160 respectively; SEQ ID NOs: 163, 165, 167 respectively; SEQ ID NOs: 170, 172, 174 respectively.

TABLE 3
Exemplary light chain CDRs and FWRs of TRBC1-targeting antigen
binding domains derived from JOVI.1

| Ab ID | VLFWR1 | VLCDR1 | VLFWR2 | VLCDR2 | VLFWR3 | VLCDR3 | VLFWR4 |
|---|---|---|---|---|---|---|---|
| mJOVI.1-L | DWMT | RSSQRL | WYLQKP | RVSNRF | GVPDRF | SQSTHV | FGGGTK |
| | QSPLSLP | VHSNGN | GQSPKL | P (SEQ ID | SGSGSG | PYT | LEIK |
| | VSLGDQ | TYLH | LIY (SEQ | NO: 109) | TDFTLKI | (SEQ ID | (SEQ ID |
| | ASISC | (SEQ ID | ID NO: | | SRVEAE | NO: 111) | NO: 112) |
| | (SEQ ID | NO: 107) | 108) | | DLGIYFC | | |
| | NO: 106) | | | | (SEQ ID | | |
| | | | | | NO: 110) | | |
| h1JOVI.1-L | DWMT | RSSQRL | WYLQKP | RVSNRF | GVPDRF | SQSTHV | FGGGTK |
| | QSPLSLP | VHSNGN | GQSPQL | P (SEQ ID | SGSGSG | PYT | VEIK |
| | VTPGEP | TYLH | LIY (SEQ | NO: 116) | TDFTLKI | (SEQ ID | (SEQ ID |
| | ASISC | (SEQ ID | ID NO: | | SRVEAE | NO: 118) | NO: 119) |
| | (SEQ ID | NO: 114) | 115) | | DVGVYF | | |
| | NO: 113) | | | | C (SEQ | | |
| | | | | | ID NO: | | |
| | | | | | 117) | | |
| h2JOVI.1-L | EVVMTQ | RSSQRL | WYQQK | RVSNRF | GIPDRFS | SQSTHV | FGGGTK |
| | SPGTLSL | VHSNGN | PGQAPR | P (SEQ ID | GSGSGT | PYT | VEIK |
| | SPGERA | TYLH | LLIY | NO: 123) | DFTLTIS | (SEQ ID | (SEQ ID |
| | TLSC | (SEQ ID | (SEQ ID | | RLEPEDF | NO: 125) | NO: 126) |
| | (SEQ ID | NO: 121) | NO: 122) | | AVYFC | | |
| | NO: 120) | | | | (SEQ ID | | |
| | | | | | NO: 124) | | |
| h3JOVI.1-L | DWMT | RSSQRL | WYQQRP | RVSNRF | GVPDRF | SQSTHV | FGGGTK |
| | QSPLSLP | VHSNGN | GQSPRL | P (SEQ ID | SGSGSG | PYT | VEIK |
| | VTLGQP | TYLH | LIY (SEQ | NO: 130) | TDFTLKI | (SEQ ID | (SEQ ID |
| | ASISC | (SEQ ID | ID NO: | | SRVEAE | NO: 132) | NO: 133) |
| | (SEQ ID | NO: 128) | 129) | | DVGVYF | | |
| | NO: 127) | | | | C (SEQ | | |
| | | | | | ID NO: | | |
| | | | | | 131) | | |
| h4JOVI.1-L | DWMT | RSSQRL | WYLQKP | RVSNRF | GVPDRF | SQSTHV | FGGGTK |
| | QTPLSLP | VHSNGN | GQSPQL | P (SEQ ID | SGSGSG | PYT | VEIK |
| | VTPGEP | TYLH | LIY (SEQ | NO: 137) | TDFTLKI | (SEQ ID | (SEQ ID |
| | ASISC | (SEQ ID | ID NO: | | SRVEAE | NO: 139) | NO: 140) |
| | (SEQ ID | NO: 135) | 136) | | DVGVYF | | |
| | NO: 134) | | | | C (SEQ | | |
| | | | | | ID NO: | | |
| | | | | | 138) | | |
| h5JOVI.1-L | DWMT | RSSQRL | WYLQKP | RVSNRF | GVPDRF | SQSTHV | FGGGTK |
| | QTPLSLS | VHSNGN | GQSPQL | P (SEQ ID | SGSGSG | PYT | VEIK |
| | VTPGQP | TYLH | LIY (SEQ | NO: 144) | TDFTLKI | (SEQ ID | (SEQ ID |
| | ASISC | (SEQ ID | ID NO: | | SRVEAE | NO: 146) | NO: 147) |
| | (SEQ ID | NO: 142) | 143) | | DVGVYF | | |
| | NO: 141) | | | | C (SEQ | | |
| | | | | | ID NO: | | |
| | | | | | 145) | | |
| L1 | DWMT | RSSQSL | WYQQRP | RVSNRF | GVPDRF | SQSTHV | FGGGTK |
| germlined-VL | QSPLSLP | VYSDGN | GQSPRL | P (SEQ ID | SGSGSG | PYT | VEIK |
| | VTLGQP | TYH | LIY (SEQ | NO: 151) | TDFTLKI | (SEQ ID | (SEQ ID |
| | ASISC | (SEQ ID | ID NO: | | SRVEAE | NO: 153) | NO: 154) |
| | (SEQ ID | NO: 149) | 150) | | DVGVYF | | |
| | NO: 148) | | | | C (SEQ | | |
| | | | | | ID NO: | | |
| | | | | | 152) | | |
| L2 | DVVMT | RSSQRL | WYQQRP | KVSNRD | GVPDRF | SQSTHV | FGGGTK |
| germlined-VL | QSPLSLP | VHSNGN | GQSPRL | S (SEQ ID | SGSGSG | PYT | VEIK |
| | VTLGQP | TYLH | LIY (SEQ | NO: 158) | TDFTLKI | (SEQ ID | (SEQ ID |
| | ASISC | (SEQ ID | ID NO: | | SRVEAE | NO: 160) | NO: 161) |
| | (SEQ ID | NO: 156) | 157) | | DVGVYF | | |
| | NO: 155) | | | | C (SEQ | | |
| | | | | | ID NO: | | |
| | | | | | 159) | | |
| L3 | DVVMT | RSSQRL | WYQQRP | RVSNRF | GVPDRF | MQSTH | FGGGTK |
| germlined-VL | QSPLSLP | VHSNGN | GQSPRL | P (SEQ ID | SGSGSG | WPYT | VEIK |
| | VTLGQP | TYLH | LIY (SEQ | NO: 165) | TDFTLKI | (SEQ ID | (SEQ ID |
| | ASISC | (SEQ ID | ID NO: | | SRVEAE | NO: 167) | NO: 168 |
| | (SEQ ID | NO: 163) | 164) | | DVGVYF | | |
| | NO: 162) | | | | C (SEQ | | |
| | | | | | ID NO: | | |
| | | | | | 166) | | |
| L1/L2/L3 | DVVMT | RSSQSL | WYQQRP | KVSNRD | GVPDRF | MQSTH | FGGGTK |
| germlined-VL | QSPLSLP | VYSDGN | GQSPRL | S (SEQ ID | SGSGSG | WPYT | VEIK |
| | VTLGQP | TYH | LIY (SEQ | NO: 172) | TDFTLKI | (SEQ ID | (SEQ ID |
| | ASISC | (SEQ ID | ID NO: | | SRVEAE | NO: 174) | NO: 175) |
| | (SEQ ID | NO: 170) | 171) | | DVGVYF | | |
| | NO: 169) | | | | C (SEQ | | |
| | | | | | ID NO: | | |
| | | | | | 173) | | |

TABLE 4
Exemplary variable regions of TRBC1-targeting antigen
binding domains

| SEQ ID | | | |
|---|---|---|---|
| NO | Ab ID | Description | Sequence |
| SEQ ID | mJOVI.1-H | JOVI.1 heavy | EVRLQQSGPDLIKPGASVKMSCKASGYTFTGYVM |
| NO: 176 | | chain variable | HWVKQRPGQGLEWIGFINPYNDDIQSNERFRGKA |
| | | region | TLTSDKSSTTAYMELSSLTSEDSAVYYCARGAGY |
| | | | NFDGAYRFFDFWGQGTTLTVSS |
| SEQ ID | h1JOVI.1-H | JOVI.1 heavy | QVQLVQSGAEVKKPGASVKVSCKASGYTFTGYV |
| NO: 177 | | chain variable | MHWVRQAPGQGLEWMGFINPYNDDIQSNERFRG |
| | | region | RVTMTSDKSTTTAYMELSSLRSEDTAVYYCARGA |
| | | humanized | GYNFDGAYRFFDFWGQGTLVTVSS |
| | | variant 1 | |
| SEQ ID | h2JOVI.1-H | JOVI.1 heavy | QVQLVQSGAEVKKPGASVKVSCKASGYTFTGYV |
| NO: 178 | | chain variable | MHWVRQAPGQGLEWMGFINPYNDDIQSNERFRG |
| | | region | WVTMTSDKSITTAYMELSRLRSDDTAVYYCARGA |
| | | humanized | GYNFDGAYRFFDFWGQGTLVTVSS |
| | | variant 2 | |
| SEQ ID | h3JOVI.1-H | JOVI.1 heavy | QVQLVQSGAEVKKPGSSVKVSCKASGYTFTGYV |
| NO: 179 | | chain variable | MHWVRQAPGQGLEWMGFINPYNDDIQSNERFRG |
| | | region | RVTITSDKSTTTAYMELSSLRSEDTAVYYCARGAG |
| | | humanized | YNFDGAYRFFDFWGQGTLVTVSS |
| | | variant 3 | |
| SEQ ID | h4JOVI.1-H | JOVI.1 heavy | QVQLVQSGAEVKKPGASVKVSCKASGYTFTGYV |
| NO: 180 | | chain variable | MHWVRQAPGQRLEWMGFINPYNDDIQSNERFRG |
| | | region | |
| | | humanized | RVTITSDKSATTAYMELSSLRSEDTAVYYCARGAG |
| | | variant 4 | YNFDGAYRFFDFWGQGTLVTVSS |
| SEQ ID | h5J0VI.1-H | JOVI.1 heavy | QVQLVQSGAEVKKPGSSVKVSCKASGYTFTGYV |
| NO: 181 | | chain variable | MHWVRQAPGQGLEWMGFINPYNDDIQSNERFRG |
| | | region | RVTITSDKSTTTAYMELSSLRSEDTAVYYCARGAG |
| | | humanized | YNFDGAYRFFDFWGQGTTVTVSS |
| | | variant 5 | |
| SEQ ID | h6J0VI.1-H | JOVI.1 heavy | QVQLVQSGAEVKKPGASVKVSCKASGYTFTGYV |
| NO: 182 | | chain variable | MHWVRQAPGQGLEWMGFINPYNDDIQSNERFRG |
| | | region | RVTMTSDKSITTAYMELSRLRSDDTAVYYCARGA |
| | | humanized | GYNFDGAYRFFDFWGQGTTVTVSS |
| | | variant 6 | |
| SEQ ID | H1 | JOVI.1 heavy | QVQLVQSGAEVKKPGSSVKVSCKASGYTFSGYAIS |
| NO: 183 | germlined- | chain variable | WVRQAPGQGLEWMGFINPYNDDIQSNERFRGRVT |
| | VH | region | ITSDKSTTTAYMELSSLRSEDTAVYYCARGAGYNF |
| | | humanized H1 | DGAYRFFDFWGQGTLVTVSS |
| SEQ ID | H2 | JOVI.1 heavy | QVQLVQSGAEVKKPGSSVKVSCKASGYTFTGYV |
| NO: 184 | germlined- | chain variable | MHWVRQAPGQGLEWMGFIIPIFGTANYAQKFQGR |
| | VH | region | VTITSDKSTTTAYMELSSLRSEDTAVYYCARGAGY |
| | | humanized H2 | NFDGAYRFFDFWGQGTLVTVS S |
| SEQ ID | H1/H2 | JOVI.1 heavy | QVQLVQSGAEVKKPGSSVKVSCKASGYTFSGYAIS |
| NO: 185 | germlined- | chain variable | WVRQAPGQGLEWMGFIIPIFGTANYAQKFQGRVTI |
| | VH | region | TSDKSTTTAYMELSSLRSEDTAVYYCARGAGYNF |
| | | humanized | DGAYRFFDFWGQGTLVTVSS |
| | | H1/H2 | |
| SEQ ID | mJOVI.1-L | JOVI.1 light | DVVMTQSPLSLPVSLGDQASISCRSSQRLVHSNGN |
| NO: 186 | | chain variable | TYLHWYLQKPGQSPKLLIYRVSNRFPGVPDRFSGS |
| | | region | GSGTDFTLKISRVEAEDLGIYFCSQSTHVPYTFGGG |
| | | | TKLEIK |
| SEQ ID | h1JOVI.1-L | JOVI.1 light | DVVMTQSPLSLPVTPGEPASISCRSSQRLVHSNGNT |
| NO: 187 | | chain variable | YLHWYLQKPGQSPQLLIYRVSNRFPGVPDRFSGSG |
| | | region | SGTDFTLKISRVEAEDVGVYFCSQSTHVPYTFGGG |
| | | humanized | TKVEIK |
| | | variant 1 | |
| SEQ ID | h2JOVI.1-L | JOVI.1 light | EVVMTQSPGTLSLSPGERATLSCRSSQRLVHSNGN |
| NO: 188 | | chain variable | TYLHWYQQKPGQAPRLLIYRVSNRFPGIPDRFSGS |
| | | region | GSGTDFTLTISRLEPEDFAVYFCSQSTHVPYTFGGG |
| | | humanized | TKVEIK |
| | | variant 2 | |
| SEQ ID | h3J0VI.1-L | JOVI.1 light | DVVMTQSPLSLPVTLGQPASISCRSSQRLVHSNGN |
| NO: 189 | | chain variable | TYLHWYQQRPGQSPRLLIYRVSNRFPGVPDRFSGS |
| | | region | GSGTDFTLKISRVEAEDVGVYFCSQSTHVPYTFGG |
| | | humanized | GTKVEIK |
| | | variant 3 | |
| SEQ ID | h4JOVI.1-L | JOVI.1 light | DVVMTQTPLSLPVTPGEPASISCRSSQRLVHSNGN |
| NO: 190 | | chain variable | TYLHWYLQKPGQSPQLLIYRVSNRFPGVPDRFSGS |
| | | region | GSGTDFTLKISRVEAEDVGVYFCSQSTHVPYTFGG |
| | | humanized | GTKVEIK |
| | | variant 4 | |
| SEQ ID | h5JOVI.1-L | JOVI.1 light | DVVMTQTPLSLSVTPGQPASISCRSSQRLVHSNGN |
| NO: 191 | | chain variable | TYLHWYLQKPGQSPQLLIYRVSNRFPGVPDRFSGS |
| | | region | GSGTDFTLKISRVEAEDVGVYFCSQSTHVPYTFGG |
| | | humanized | GTKVEIK |
| | | variant 5 | |
| SEQ ID | L1 | JOVI.1 light | DVVMTQSPLSLPVTLGQPASISCRSSQSLVYSDGN |
| NO: 192 | germlined- | chain variable | TYHWYQQRPGQSPRLLIYRVSNRFPGVPDRFSGSG |
| | VL | region | SGTDFTLKISRVEAEDVGVYFCSQSTHVPYTFGGG |
| | | humanized Li | TKVEIK |
| SEQ ID | L2 | JOVI.1 light | DVVMTQSPLSLPVTLGQPASISCRSSQRLVHSNGN |
| NO: 193 | germlined- | chain variable | TYLHWYQQRPGQSPRLLIYKVSNRDSGVPDRFSGS |
| | VL | region | GSGTDFTLKISRVEAEDVGVYFCSQSTHVPYTFGG |
| | | humanized L2 | GTKVEIK |
| SEQ ID | L3 | JOVI.1 light | DVVMTQSPLSLPVTLGQPASISCRSSQRLVHSNGN |
| NO: 194 | germlined- | chain variable | TYLHWYQQRPGQSPRLLIYRVSNRFPGVPDRFSGS |
| | VL | region | GSGTDFTLKISRVEAEDVGVYFCMQSTHWPYTFG |
| | | humanized L3 | GGTKVEIK |
| SEQ ID | L1/L2/L3 | JOVI.1 light | DVVMTQSPLSLPVTLGQPASISCRSSQSLVYSDGN |
| NO: 195 | germlined- | chain variable | TYHWYQQRPGQSPRLLIYKVSNRDSGVPDRFSGS |
| | VL | region | GSGTDFTLKISRVEAEDVGVYFCMQSTHWPYTFG |
| | | humanized | GGTKVEIK |
| | | L1/L2/L3 | |

TABLE 5
Exemplary TRBC1-targeting antigen binding domains/antibody molecules
or functional fragments thereof

| SEQ ID | | | |
|---|---|---|---|
| NO | Ab ID | Description | Sequence |
| SEQ ID | Ch(anti- | Anti-TRBC1 | EVRLQQSGPDLIKPGASVKMSCKASGYTFTGYVM |
| NO: 196 | TRBC1) | heavy chain | HWVKQRPGQGLEWIGFINPYNDDIQSNERFRGKA |
| | HC N297A | | TLTSDKSSTTAYMELSSLTSEDSAVYYCARGAGY |
| | | | NFDGAYRFFDFWGQGTTLTVSSASTKGPSVFPLAP |
| | | | SSKSTSGGTAALGCLVKDYFPEPVTVSWNSGALTS |
| | | | GVHTFPAVLQSSGLYSLSSVVTVPSSSLGTQTYICN |
| | | | VNHKPSNTKVDKRVEPKSCDKTHTCPPCPAPELLG |
| | | | GPSVFLFPPKPKDTLMISRTPEVTCVVVDVSHEDPE |
| | | | VKFNWYVDGVEVHNAKTKPREEQYASTYRVVSV |
| | | | LTVLHQDWLNGKEYKCKVSNKALPAPIEKTISKA |
| | | | KGQPREPQVYTLPPCREEMTKNQVSLWCLVKGFY |
| | | | PSDIAVEWESNGQPENNYKTTPPVLDSDGSFFLYS |
| | | | KLTVDKSRWQQGNVFSCSVMHEALHNHYTQKSL |
| | | | SLSPGK |
| SEQ ID | Ch(anti- | Anti-TRBC1 | EVRLQQSGPDLIKPGASVKMSCKASGYTFTGYVM |
| NO: 197 | TRBC1) | heavy chain | HWVKQRPGQGLEWIGFINPYNDDIQSNERFRGKA |
| | HC | | TLTSDKSSTTAYMELSSLTSEDSAVYYCARGAGY |
| | | | NFDGAYRFFDFWGQGTTLTVSSASTKGPSVFPLAP |
| | | | SSKSTSGGTAALGCLVKDYFPEPVTVSWNSGALTS |
| | | | GVHTFPAVLQSSGLYSLSSVVTVPSSSLGTQTYICN |
| | | | VNHKPSNTKVDKRVEPKSCDKTHTCPPCPAPELLG |
| | | | GPSVFLFPPKPKDTLMISRTPEVTCVVVDVSHEDPE |
| | | | VKFNWYVDGVEVHNAKTKPREEQYNSTYRVVSV |
| | | | LTVLHQDWLNGKEYKCKVSNKALPAPIEKTISKA |
| | | | KGQPREPQVYTLPPCREEMTKNQVSLWCLVKGFY |
| | | | PSDIAVEWESNGQPENNYKTTPPVLDSDGSFFLYS |
| | | | KLTVDKSRWQQGNVFSCSVMHEALHNHYTQKSL |
| | | | SLSPGK |
| SEQ ID | Ch(anti- | Anti-TRBC1 | DVVMTQSPLSLPVSLGDQASISCRSSQRLVHSNGN |
| NO: 198 | TRBC1) | light chain, | TYLHWYLQKPGQSPKLLIYRVSNRFPGVPDRFSGS |
| | LC | e.g., a LC | GSGTDFTLKISRVEAEDLGIYFCSQSTHVPYTFGGG |
| | | Fab | TKLEIKRTVAAPSVFIFPPSDEQLKSGTASVVCLLN |
| | | | NFYPREAKVQWKVDNALQSGNSQESVTEQDSKDS |
| | | | TYSLSSTLTLSKADYEKHKVYACEVTHQGLSSPVT |
| | | | KSFNRGEC |
| SEQ ID | Ch(anti- | Anti-TRBC1 | EVRLQQSGPDLIKPGASVKMSCKASGYTFTGYVM |
| NO: 199 | TRBC1) | heavy chain, | HWVKQRPGQGLEWIGFINPYNDDIQSNERFRGKA |
| | HC | e.g., a HC | TLTSDKSSTTAYMELSSLTSEDSAVYYCARGAGY |
| | | Fab | NFDGAYRFFDFWGQGTTLTVSSASTKGPSVFPLAP |
| | | | SSKSTSGGTAALGCLVKDYFPEPVTVSWNSGALTS |
| | | | GVHTFPAVLQSSGLYSLSSVVTVPSSSLGTQTYICN |
| | | | VNHKPSNTKVDKRVEPKSC |
| SEQ ID | ahTRBC1_ | Anti-TRBC1 | METDTLLLWVLLLWVPGSTGQVQLVQSGAEVKK |
| NO: 200 | Jovil_ | heavy chain | PGSSVKVSCKASGYTFTGYVMHWVRQAPGQGLE |
| | Hum5_VH- | | WMGFINPYNDDIQSNERFRGRVTITSDKSTTTAYM |
| | hCHIg_ | | ELSSLRSEDTAVYYCARGAGYNFDGAYRFFDFWG |
| | Hole_Cys- | | QGTLVTVSSASTKGPSVFPLAPSSKSTSGGTAALG |
| | Blank | | CLVKDYFPEPVTVSWNSGALTSGVHTFPAVLQSSG |
| | | | LYSLSSVVTVPSSSLGTQTYICNVNHKPSNTKVDK |
| | | | RVEPKSCDKTHTCPPCPAPELLGGPSVFLFPPKPKD |
| | | | TLMISRTPEVTCVVVDVSHEDPEVKFNWYVDGVE |
| | | | VHNAKTKPREEQYNSTYRVVSVLTVLHQDWLNG |
| | | | KEYKCKVSNKALPAPIEKTISKAKGQPREPQVCTL |
| | | | PPSREEMTKNQVSLSCAVKGFYPSDIAVEWESNGQ |
| | | | PENNYKTTPPVLDSDGSFFLVSKLTVDKSRWQQG |
| | | | NVFSCSVMHEALHNHYTQKSLSLSPGK |
| SEQ ID | ahTRBC_ | Anti-TRBC1 | METDTLLLWVLLLWVPGSTGQVQLVQSGAEVKK |
| NO: 201 | Jovil_ | heavy chain | PGSSVKVSCKASGYTFTGYVMHWVRQAPGQGLE |
| | Hum5_VH- | | WMGFINPYNDDIQSNERFRGRVTITSDKSTTTAYM |
| | hCHIg- | | ELSSLRSEDTAVYYCARGAGYNFDGAYRFFDFWG |
| | Blank | | QGTLVTVSSASTKGPSVFPLAPSSKSTSGGTAALG |
| | | | CLVKDYFPEPVTVSWNSGALTSGVHTFPAVLQSSG |
| | | | LYSLSSVVTVPSSSLGTQTYICNVNHKPSNTKVDK |
| | | | RVEPKSCDKTHTCPPCPAPELLGGPSVFLFPPKPKD |
| | | | TLMISRTPEVTCVVVDVSHEDPEVKFNWYVDGVE |
| | | | VHNAKTKPREEQYNSTYRVVSVLTVLHQDWLNG |
| | | | KEYKCKVSNKALPAPIEKTISKAKGQPREPQVYTL |
| | | | PPSREEMTKNQVSLTCLVKGFYPSDIAVEWESNGQ |
| | | | PENNYKTTPPVLDSDGSFFLYSKLTVDKSRWQQG |
| | | | NVFSCSVMHEALHNHYTQKSLSLSPGK |
| SEQ ID | a_hTRBC1_ | Anti-TRBC1 | METDTLLLWVLLLWVPGSTGDVVMTQSPLSLPVT |
| NO: 202 | Jovil_ | light chain | LGQPASISCRSSQRLVHSNGNTYLHWYQQRPGQSP |
| | Hum3 VL- | | RLLIYRVSNRFPGVPDRFSGSGSGTDFTLKISRVEA |
| | hCLIgvk- | | EDVGVYFCSQSTHVPYTFGGGTKVEIKRTVAAPSV |
| | Blank | | FIFPPSDEQLKSGTASVVCLLNNFYPREAKVQWKV |
| | | | DNALQSGNSQESVTEQDSKDSTYSLSSTLTLSKAD |
| | | | YEKHKVYACEVTHQGLSSPVTKSFNRGEC |

TABLE 6
Exemplary antibody molecules or functional fragments thereof
that bind to TRBC1

| SEQ ID NO | Description | Sequence |
|---|---|---|
| BJM0772 | | |
| SEQ ID NO: | anti-TRBC1 | QVQLVQSGAEVKKPGSSVKVSCKASGYTFTGYVMHWVRQAPG |
| 203 | HC | QGLEWMGFINPYNDDIQSNERFRGRVTITSDKSTTTAYMELSSLR |
| | | SEDTAVYYCARGAGYNFDGAYRFFDFWGQGTLVTVSSASTKGP |
| | | SVFPLAPSSKSTSGGTAALGCLVKDYFPEPVTVSWNSGALTSGV |
| | | HTFPAVLQSSGLYSLSSVVTVPSSSLGTQTYICNVNHKPSNTKVD |
| | | KRVEPKSCDKTHTCPPCPAPELLGGPSVFLFPPKPKDTLMISRTPE |
| | | VTCVVVDVSHEDPEVKFNWYVDGVEVHNAKTKPREEQYASTY |
| | | RVVSVLTVLHQDWLNGKEYKCKVSNKALPAPIEKTISKAKGQPR |
| | | EPQVYTLPPCREEMTKNQVSLWCLVKGFYPSDIAVEWESNGQPE |
| | | nnykttppvldsdgsfflyskltvdksrwqqgnvfscsvmhea |
| | | LHNHYTQKSLSLSPGK |
| SEQ ID NO: | anti-TRBC1 | DVVMTQSPLSLPVTLGQPASISCRSSQRLVHSNGNTYLHWYQQR |
| 204 | LC | PGQSPRLLIYRVSNRFPGVPDRFSGSGSGTDFTLKISRVEAEDVG |
| | | VYFCSQSTHVPYTFGGGTKVEIKRTVAAPSVFIFPPSDEQLKSGT |
| | | ASVVCLLNNFYPREAKVQWKVDNALQSGNSQESVTEQDSKDST |
| | | YSLSSTLTLSKADYEKHKVYACEVTHQGLSSPVTKSFNRGEC |
| BJM1042 | | |
| SEQ ID NO: | anti-TRBC1 | QVQLVQSGAEVKKPGSSVKVSCKASGYTFTGYVMHWVRQAPG |
| 205 | HC | QGLEWMGFIIPIFGTANYAQKFQGRVTITSDKSTTTAYMELSSLR |
| | | SEDTAVYYCARGAGYNFDGAYRFFDFWGQGTLVTVSSASTKGP |
| | | SVFPLAPSSKSTSGGTAALGCLVKDYFPEPVTVSWNSGALTSGV |
| | | HTFPAVLQSSGLYSLSSVVTVPSSSLGTQTYICNVNHKPSNTKVD |
| | | KRVEPKSCDKTHTCPPCPAPELLGGPSVFLFPPKPKDTLMISRTPE |
| | | VTCVVVDVSHEDPEVKFNWYVDGVEVHNAKTKPREEQYASTY |
| | | RVVSVLTVLHQDWLNGKEYKCKVSNKALPAPIEKTISKAKGQPR |
| | | EPQVYTLPPCREEMTKNQVSLWCLVKGFYPSDIAVEWESNGQPE |
| | | nnykttppvldsdgsfflyskltvdksrwqqgnvfscsvmhea |
| | | LHNHYTQKSLSLSPGK |
| SEQ ID NO: | anti-TRBC1 | DVVMTQSPLSLPVTLGQPASISCRSSQRLVHSNGNTYLHWYQQR |
| 206 | LC | PGQSPRLLIYRVSNRFPGVPDRFSGSGSGTDFTLKISRVEAEDVG |
| | | VYFCSQSTHVPYTFGGGTKVEIKRTVAAPSVFIFPPSDEQLKSGT |
| | | ASVVCLLNNFYPREAKVQWKVDNALQSGNSQESVTEQDSKDST |
| | | YSLSSTLTLSKADYEKHKVYACEVTHQGLSSPVTKSFNRGEC |
| BJM1052 | | |
| SEQ ID NO: | anti-TRBC1 | QVQLVQSGAEVKKPGSSVKVSCKASGYTFTGYVMHWVRQAPG |
| 207 | HC | QGLEWMGFINPYNDDIQSNERFRGRVTITSDKSTTTAYMELSSLR |
| | | SEDTAVYYCARGAGYNFDGAYRFFDFWGQGTLVTVSSASTKGP |
| | | SVFPLAPSSKSTSGGTAALGCLVKDYFPEPVTVSWNSGALTSGV |
| | | HTFPAVLQSSGLYSLSSVVTVPSSSLGTQTYICNVNHKPSNTKVD |
| | | KRVEPKSCDKTHTCPPCPAPELLGGPSVFLFPPKPKDTLMISRTPE |
| | | VTCVVVDVSHEDPEVKFNWYVDGVEVHNAKTKPREEQYASTY |
| | | RVVSVLTVLHQDWLNGKEYKCKVSNKALPAPIEKTISKAKGQPR |
| | | EPQVYTLPPCREEMTKNQVSLWCLVKGFYPSDIAVEWESNGQPE |
| | | NNYKTTPPVLDSDGSFFLYSKLTVDKSRWQQGNVFSCSVMHEA |
| | | LHNHYTQKSLSLSPGK |
| SEQ ID NO: | anti-TRBC1 | DVVMTQSPLSLPVTLGQPASISCRSSQRLVHSNGNTYLHWYQQR |
| 208 | LC | PGQSPRLLIYRVSNRFPGVPDRFSGSGSGTDFTLKISRVEAEDVG |
| | | VYFCSQSTHVPYTFGGGTKVEIKRTVAAPSVFIFPPSDEQLKSGT |
| | | ASVVCLLNNFYPREAKVQWKVDNALQSGNSQESVTEQDSKDST |
| | | YSLSSTLTLSKADYEKHKVYACEVTHQGLSSPVTKSFNRGEC |
| BJM1038 | | |
| SEQ ID NO: | anti-TRBC1 | QVQLVQSGAEVKKPGSSVKVSCKASGYTFTGYVMHWVRQAPG |
| 209 | HC | QGLEWMGFIIPIFGTANYAQKFQGRVTITSDKSTTTAYMELSSLR |
| | | SEDTAVYYCARGAGYNFDGAYRFFDFWGQGTLVTVSSASTKGP |
| | | SVFPLAPSSKSTSGGTAALGCLVKDYFPEPVTVSWNSGALTSGV |
| | | HTFPAVLQSSGLYSLSSVVTVPSSSLGTQTYICNVNHKPSNTKVD |
| | | KRVEPKSCDKTHTCPPCPAPELLGGPSVFLFPPKPKDTLMISRTPE |
| | | VTCVVVDVSHEDPEVKFNWYVDGVEVHNAKTKPREEQYASTY |
| | | RVVSVLTVLHQDWLNGKEYKCKVSNKALPAPIEKTISKAKGQPR |
| | | EPQVYTLPPCREEMTKNQVSLWCLVKGFYPSDIAVEWESNGQPE |
| | | nnykttppvldsdgsfflyskltvdksrwqqgnvfscsvmhea |
| | | LHNHYTQKSLSLSPGK |
| SEQ ID NO: | anti-TRBC1 | DVVMTQSPLSLPVTLGQPASISCRSSQRLVHSNGNTYLHWYQQR |
| 210 | LC | PGQSPRLLIYRVSNRFPGVPDRFSGSGSGTDFTLKISRVEAEDVG |
| | | VYFCSQSTHVPYTFGGGTKVEIKRTVAAPSVFIFPPSDEQLKSGT |
| | | ASVVCLLNNFYPREAKVQWKVDNALQSGNSQESVTEQDSKDST |
| | | YSLSSTLTLSKADYEKHKVYACEVTHQGLSSPVTKSFNRGEC |
| BJM1048 | | |
| SEQ ID NO: | anti-TRBC1 | QVQLVQSGAEVKKPGSSVKVSCKASGYTFTGYVMHWVRQAPG |
| 211 | HC | QGLEWMGFINPYNDDIQSNERFRGRVTITSDKSTTTAYMELSSLR |
| | | SEDTAVYYCARGAGYNFDGAYRFFDFWGQGTLVTVSSASTKGP |
| | | SVFPLAPSSKSTSGGTAALGCLVKDYFPEPVTVSWNSGALTSGV |
| | | HTFPAVLQSSGLYSLSSVVTVPSSSLGTQTYICNVNHKPSNTKVD |
| | | KRVEPKSCDKTHTCPPCPAPELLGGPSVFLFPPKPKDTLMISRTPE |
| | | VTCVVVDVSHEDPEVKFNWYVDGVEVHNAKTKPREEQYASTY |
| | | RVVSVLTVLHQDWLNGKEYKCKVSNKALPAPIEKTISKAKGQPR |
| | | EPQVYTLPPCREEMTKNQVSLWCLVKGFYPSDIAVEWESNGQPE |
| | | NNYKTTPPVLDSDGSFFLYSKLTVDKSRWQQGNVFSCSVMHEA |
| | | LHNHYTQKSLSLSPGK |
| SEQ ID NO: | anti-TRBC1 | DVVMTQSPLSLPVTLGQPASISCRSSQRLVHSNGNTYLHWYQQR |
| 212 | LC | PGQSPRLLIYRVSNRFPGVPDRFSGSGSGTDFTLKISRVEAEDVG |
| | | VYFCSQSTHVPYTFGGGTKVEIKRTVAAPSVFIFPPSDEQLKSGT |
| | | ASVVCLLNNFYPREAKVQWKVDNALQSGNSQESVTEQDSKDST |
| | | YSLSSTLTLSKADYEKHKVYACEVTHQGLSSPVTKSFNRGEC |

### TCR Targeting TRBC2

In another aspect, provided herein is an TCRB2 targeting molecule that has an antigen binding domain that binds to TRBC2, comprising one or more CDRs, framework regions, variable regions, or antigen binding domains disclosed in any of Tables 7-10, or a sequence having at least 85%, 90%, 95%, or 99% identity thereto.

In some embodiments, the antibody molecule, or fragment thereof, that binds to TRBC2 comprises one or more CDRs (e.g., VHCDR1, VHCDR2, VHCDR3, VLCDR1, VLCDR2, and/or VLCDR3) disclosed in Table 7 or Table 8, or a sequence having at least 85%, 90%, 95%, or 99% identity thereto. In some embodiments, the antibody molecule, or fragment thereof, that binds to TRBC2 comprises one or more framework regions (e.g., VHFWR1, VHFWR2, VHFWR3, VHFWR4, VLFWR1, VLFWR2, VLFWR3, and/or VLFWR4) disclosed in Table 7 or Table 8, or a sequence having at least 85%, 90%, 95%, or 99% identity thereto. In some embodiments, the antibody molecule, or fragment thereof, that binds to TRBC2 comprises a VH and/or a VL disclosed in Table 9, or a sequence having at least 85%, 90%, 95%, or 99% identity thereto. In some embodiments, the antibody molecule, or fragment thereof, that binds to TRBC2 comprises an amino acid sequence disclosed in Table 10, or a sequence having at least 85%, 90%, 95%, or 99% identity thereto.

In some embodiments, the antibody molecule, or fragment thereof, that binds to TRBC2 comprises a VH comprising a heavy chain complementarity determining region 1 (VHCDR1), a VHCDR2, and a VHCDR3, and a VL comprising a light chain complementarity determining region 1 (VLCDR1), a VLCDR2, and a VLCDR3.

In some embodiments, the VHCDR1, VHCDR2, and VHCDR3 comprise the amino acid sequences of SEQ ID NOs: 305, 306, and 307, respectively (or a sequence having at least 85%, 90%, 95%, or 99% identity thereto). In some embodiments, the VHCDR1, VHCDR2, and VHCDR3 comprise the amino acid sequences of SEQ ID NOs: 271, 273, and 275, respectively (or a sequence having at least 85%, 90%, 95%, or 99% identity thereto). In some embodiments, the VHCDR1, VHCDR2, and VHCDR3 comprise the amino acid sequences of SEQ ID NOs: 278, 280, and 282, respectively (or a sequence having at least 85%, 90%, 95%, or 99% identity thereto). In some embodiments, the VHCDR1, VHCDR2, and VHCDR3 comprise the amino acid sequences of SEQ ID NOs: 222, 224, and 226, respectively (or a sequence having at least 85%, 90%, 95%, or 99% identity thereto). In some embodiments, the VHCDR1, VHCDR2, and VHCDR3 comprise the amino acid sequences of SEQ ID NOs: 229, 231, and 233, respectively (or a sequence having at least 85%, 90%, 95%, or 99% identity thereto). In some embodiments, the VHCDR1, VHCDR2, and VHCDR3 comprise the amino acid sequences of SEQ ID NOs: 236, 238, and 240, respectively (or a sequence having at least 85%, 90%, 95%, or 99% identity thereto). In some embodiments, the VHCDR1, VHCDR2, and VHCDR3 comprise the amino acid sequences of SEQ ID NOs: 243, 245, and 247, respectively (or a sequence having at least 85%, 90%, 95%, or 99% identity thereto. In some embodiments, the VHCDR1, VHCDR2, and VHCDR3 comprise the amino acid sequences of SEQ ID NOs: 250, 252, and 253, respectively (or a sequence having at least 85%, 90%, 95%, or 99% identity thereto. In some embodiments, the VHCDR1, VHCDR2, and VHCDR3 comprise the amino acid sequences of SEQ ID NOs: 257, 259, and 261, respectively (or a sequence having at least 85%, 90%, 95%, or 99% identity thereto. In some embodiments, the VHCDR1, VHCDR2, and VHCDR3 comprise the amino acid sequences of SEQ ID NOs: 264, 266, and 268, respectively (or a sequence having at least 85%, 90%, 95%, or 99% identity thereto. In some embodiments, the VHCDR1, VHCDR2, and VHCDR3 comprise the amino acid sequences of SEQ ID NOs: 271, 273, and 275, respectively (or a sequence having at least 85%, 90%, 95%, or 99% identity thereto. In some embodiments, the VHCDR1, VHCDR2, and VHCDR3 comprise the amino acid sequences of SEQ ID NOs: 278, 280, and 282, respectively (or a sequence having at least 85%, 90%, 95%, or 99% identity thereto. In some embodiments, the VHCDR1, VHCDR2, and VHCDR3 comprise the amino acid sequences of SEQ ID NOs: 285, 287, and 289, respectively (or a sequence having at least 85%, 90%, 95%, or 99% identity thereto. In some embodiments, the VHCDR1, VHCDR2, and VHCDR3 comprise the amino acid sequences of SEQ ID NOs: 292, 294, and 296, respectively (or a sequence having at least 85%, 90%, 95%, or 99% identity thereto. In some embodiments, the VHCDR1, VHCDR2, and VHCDR3 comprise the amino acid sequences of SEQ ID NOs: 299, 301, and 303, respectively (or a sequence having at least 85%, 90%, 95%, or 99% identity thereto.

In some embodiments, the VLCDR1, VLCDR2, and VLCDR3 comprise a consensus amino acid sequences of SEQ ID NOs: 322, 323, and 324, respectively (or a sequence having at least 85%, 90%, 95%, or 99% identity thereto). In some embodiments, the VLCDR1, VLCDR2, and VLCDR3 comprise the amino acid sequences of SEQ ID NOs: 309, 311, and 313, respectively (or a sequence having at least 85%, 90%, 95%, or 99% identity thereto). In some embodiments, the VLCDR1, VLCDR2, and VLCDR3 comprise the amino acid sequences of SEQ ID NOs: 316, 318, and 320, respectively (or a sequence having at least 85%, 90%, 95%, or 99% identity thereto).

In some embodiments, the VHCDR1, VHCDR2, VHCDR3, VLCDR1, VLCDR2, and VLCDR3 comprise the amino acid sequences of SEQ ID NOs: 305, 306, 307, 322, 323, and 324, respectively (or a sequence having at least 85%, 90%, 95%, or 99% identity thereto). In some embodiments, the VHCDR1, VHCDR2, VHCDR3, VLCDR1, VLCDR2, and VLCDR3 comprise the amino acid sequences of SEQ ID NOs: 271, 273, 275, 309, 311, and 313, respectively (or a sequence having at least 85%, 90%, 95%, or 99% identity thereto). In some embodiments, the VHCDR1, VHCDR2, VHCDR3, VLCDR1, VLCDR2, and VLCDR3 comprise the amino acid sequences of SEQ ID NOs: 278, 280, 282, 309, 311, and 313, respectively (or a sequence having at least 85%, 90%, 95%, or 99% identity thereto). In some embodiments, the VHCDR1, VHCDR2, VHCDR3, VLCDR1, VLCDR2, and VLCDR3 comprise the amino acid sequences of: SEQ ID NOs: 285, 287, 289, 309, 311, and 313 respectively (or a sequence having at least 85%, 90%, 95%, or 99% identity thereto); SEQ ID NOs: 292, 294, 296, 309, 311, and 313 respectively (or a sequence having at least 85%, 90%, 95%, or 99% identity thereto); SEQ ID NOs: 299, 301, 303, 309, 311, and 313 respectively (or a sequence having at least 85%, 90%, 95%, or 99% identity thereto); SEQ ID NOs: 271, 273, 275, 316, 318, and 320, respectively (or a sequence having at least 85%, 90%, 95%, or 99% identity thereto); SEQ ID NOs: 271, 273, 275, 316, 318, and 320, respectively (or a sequence having at least 85%, 90%, 95%, or 99% identity thereto); SEQ ID NOs: 271, 273, 275, 316, 318, and 320, respectively (or a sequence having at least 85%, 90%, 95%, or 99% identity thereto); SEQ ID NOs: 271, 273, 275, 316, 318, and 320, respectively (or a sequence having at least 85%, 90%, 95%, or 99% identity thereto); SEQ ID NOs: 271, 273, 275, 316, 318, and 320, respectively (or a sequence having at least 85%, 90%, 95%, or 99% identity thereto);

In some embodiments, the TCR antigen binding domain may comprise any one of the heavy chain variable domain (VH) sequences selected from SEQ ID NOs: 325-331. In some embodiments, the TCR antigen binding domain may comprise any one of the heavy chain variable domain (VH) sequences selected from SEQ ID NOs: 332, 333, 334, 335. In some embodiments, the TCR antigen binding domain may comprise any one of the light chain variable domain (VL) sequences selected from SEQ ID NOs: 337, 338.

In some embodiments, an antigen binding domain may comprise a VH domain SEQ ID NO: 332, and a VL of SEQ ID NO: 337 or 338. In some embodiments, the antigen binding domain may comprise a VH domain SEQ ID NO: 333, and a VL of SEQ ID NO: 337 or 338. In some embodiments, the antigen binding domain may comprise a VH domain SEQ ID NO: 334, and a VL of SEQ ID NO: 337 or 338. In some embodiments, the antigen binding domain may comprise a VH domain SEQ ID NO: 335, and a VL of SEQ ID NO: 337 or 338. In some embodiments, the antigen binding domain may comprise a VH domain SEQ ID NO: 336, and a VL of SEQ ID NO: 337 or 338.

In some embodiments, the antigen binding domains comprise an ScFv, that can bind to a TCRB2 antigen. In some embodiments, the ScFv comprises a sequence comprises a sequence of SEQ ID NO: 336. In some embodiments, the ScFv comprises a sequence comprises a sequence of SEQ ID NO: 339. In some embodiments, the ScFv comprises a sequence comprises a sequence of SEQ ID NO: 340. In some embodiments, the ScFv comprises a sequence comprises a sequence of SEQ ID NO: 341. In some embodiments, the ScFv comprises a sequence comprises a sequence of SEQ ID NO: 342. In some embodiments, the ScFv comprises a sequence comprises a sequence of SEQ ID NO: 343.

TABLE 7
Exemplary heavy chain CDRs and FWRs of TRBC2-targeting
antigen binding domains

| Ab ID | VHFWR1 | VHCDR1 | VHFWR2 | VHCDR2 | VHFWR3 | VHCDR3 | VHFWR4 |
|---|---|---|---|---|---|---|---|
| JVD3- | QVQLVQ | GFVMH | WVRQAP | FINPYND | RVTMTS | GNGMW | WGQGTL |
| VH | SGAEVK | (SEQ ID | GQGLEW | DIQSNER | DKSTTTA | FDGAYR | VTVSS |
| | KPGASVK | NO: 222) | MG (SEQ | FRG | YMELSSL | FFDF | (SEQ ID |
| | VSCKASG | | ID NO: | (SEQ ID | RSEDTAV | (SEQ ID | NO: 227) |
| | YTFP | | 223) | NO: 224) | YYCAR | NO: 226) | |
| | (SEQ ID | | | | (SEQ ID | | |
| | NO: 221) | | | | NO: 225) | | |
| JVD4- | QVQLVQ | GYVMH | WVRQAP | FINPYND | RVTMTS | GNGKNF | WGQGTL |
| VH | SGAEVK | (SEQ ID | GQGLEW | DIQSNER | DKSTTTA | DGAYRF | VTVSS |
| | KPGASVK | NO: 229) | MG (SEQ | FRG | YMELSSL | FDF (SEQ | (SEQ ID |
| | VSCKASG | | ID NO: | (SEQ ID | RSEDTAV | ID NO: | NO: 234) |
| | YPFH | | 230) | NO: 231) | YYCAR | 233) | |
| | (SEQ ID | | | | (SEQ ID | | |
| | NO: 228) | | | | NO: 232) | | |
| JVD5- | QVQLVQ | GYVMH | WVRQAP | FINPYND | RVTMTS | GNGKW | WGQGTL |
| VH | SGAEVK | (SEQ ID | GQGLEW | DIQSNER | DKSTTTA | FDGAYR | VTVSS |
| | KPGASVK | NO: 236) | MG (SEQ | FRG | YMELSSL | FFDF | (SEQ ID |
| | VSCKASG | | ID NO: | (SEQ ID | RSEDTAV | (SEQ ID | NO: 241) |
| | YTYP | | 237) | NO: 238) | YYCAR | NO: 240) | |
| | (SEQ ID | | | | (SEQ ID | | |
| | NO: 235) | | | | NO: 239) | | |
| JVD6- | QVQLVQ | GYHMH | WVRQAP | FINPYND | RVTMTS | GNGKW | WGQGTL |
| VH | SGAEVK | (SEQ ID | GQGLEW | DIQSNER | DKSTTTA | GDGAYR | VTVSS |
| | KPGASVK | NO: 243) | MG (SEQ | FRG | YMELSSL | FFDF | (SEQ ID |
| | VSCKASN | | ID NO: | (SEQ ID | RSEDTAV | (SEQ ID | NO: 248) |
| | QNFH | | 244) | NO: 245) | YYCAR | NO: 247) | |
| | (SEQ ID | | | | (SEQ ID | | |
| | NO: 242) | | | | NO: 246) | | |
| JVD7- | QVQLVQ | GFYMH | WVRQAP | FINPYND | RVTMTS | GNGKW | WGQGTL |
| VH | SGAEVK | (SEQ ID | GQGLEW | DIQSNER | DKSTTTA | GDGAYR | VTVSS |
| | KPGASVK | NO: 250) | MG (SEQ | FRG | YMELSSL | FFDF | (SEQ ID |
| | VSCKASS | | ID NO: | (SEQ ID | RSEDTAV | (SEQ ID | NO: 255) |
| | QNFH | | 251) | NO: 252) | YYCAR | NO: 254) | |
| | (SEQ ID | | | | (SEQ ID | | |
| | NO: 249) | | | | NO: 253) | | |
| JVD8- | QVQLVQ | GYKMH | WVRQAP | FINPYND | RVTMTS | GNGKW | WGQGTL |
| VH | SGAEVK | (SEQ ID | GQGLEW | DIQSNER | DKSTTTA | GDGAYR | VTVSS |
| | KPGASVK | NO: 257) | MG (SEQ | FRG | YMELSSL | FFDF | (SEQ ID |
| | VSCKASY | | ID NO: | (SEQ ID | RSEDTAV | (SEQ ID | NO: 262) |
| | QDFH | | 258) | NO: 259) | YYCAR | NO: 261) | |
| | (SEQ ID | | | | (SEQ ID | | |
| | NO: 256) | | | | NO: 260) | | |
| JVD9- | QVQLVQ | GFYMH | WVRQAP | FINPYND | RVTMTS | GNGKW | WGQGTL |
| VH | SGAEVK | (SEQ ID | GQGLEW | DIQSNER | DKSTTTA | GDGAYR | VTVSS |
| | KPGASVK | NO: 264) | MG (SEQ | FRG | YMELSSL | FFDF | (SEQ ID |
| | VSCKASG | | ID NO: | (SEQ ID | RSEDTAV | (SEQ ID | NO: 269) |
| | YNFH | | 265) | NO: 266) | YYCAR | NO: 268) | |
| | (SEQ ID | | | | (SEQ ID | | |
| | NO: 263) | | | | NO: 267) | | |
| BKM0097 | QVQLVQ | GYPMH | WVRQAP | FINPYND | RVTMTS | GNGKW | WGQGTL |
| anti- | SGAEVK | (SEQ ID | GQGLEW | DIQSNER | DKSTTTA | GDGAYR | VTVSS |
| TRBC2 | KPGASVK | NO: 271) | MG (SEQ | FRG | YMELSSL | FFDF | (SEQ ID |
| VH; | VSCKAST | | ID NO: | (SEQ ID | RSEDTAV | (SEQ ID | NO: 276) |
| BJM1184 | SGFH | | 272) | NO: 273) | YYCAR | NO: 275) | |
| VH | (SEQ ID | | | | (SEQ ID | | |
| | NO: 270) | | | | NO: 274) | | |
| BKM0098 | QVQLVQ | GYHMH | WVRQAP | FINPYND | RVTMTS | GNGKW | WGQGTL |
| anti- | SGAEVK | (SEQ ID | GQGLEW | DIQSNER | DKSTTTA | GDGAYR | VTVSS |
| TRBC2 | KPGASVK | NO: 278) | MG (SEQ | FRG | YMELSSL | FFDF | (SEQ ID |
| VH; | VSCKASP | | ID NO: | (SEQ ID | RSEDTAV | (SEQ ID | NO: 283) |
| BJM1185 | RGFH | | 279) | NO: 280) | YYCAR | NO: 282) | |
| VH | (SEQ ID | | | | (SEQ ID | | |
| | NO: 277) | | | | NO: 281) | | |
| BJM1186 | QVQLVQ | GYAMH | WVRQAP | FINPYND | RVTMTS | GNGKW | WGQGTL |
| VH | SGAEVK | (SEQ ID | GQGLEW | DIQSNER | DKSTTTA | GDGAYR | VTVSS |
| | KPGASVK | NO: 285) | MG (SEQ | FRG | YMELSSL | FFDF | (SEQ ID |
| | VSCKASF | | ID NO: | (SEQ ID | RSEDTAV | (SEQ ID | NO: 290) |
| | QDFH | | 286) | NO: 287) | YYCAR | NO: 289) | |
| | (SEQ ID | | | | (SEQ ID | | |
| | NO: 284) | | | | NO: 288) | | |
| BJM1187 | QVQLVQ | GFAMH | WVRQAP | FINPYND | RVTMTS | GNGKW | WGQGTL |
| VH | SGAEVK | (SEQ ID | GQGLEW | DIQSNER | DKSTTTA | GDGAYR | VTVSS |
| | KPGASVK | NO: 292) | MG (SEQ | FRG | YMELSSL | FFDF | (SEQ ID |
| | VSCKASS | | ID NO: | (SEQ ID | RSEDTAV | (SEQ ID | NO: 297) |
| | KDFH | | 293) | NO: 294) | YYCAR | NO: 296) | |
| | (SEQ ID | | | | (SEQ ID | | |
| | NO: 291) | | | | NO: 295) | | |
| BC2_YR3_ | QVQLVQ | GYHMH | WVRQAP | FINPYND | RVTMTS | GNGKW | WGQGTL |
| B11- | SGAEVK | (SEQ ID | GQGLEW | DIQSNER | DKSTTTA | GDGAYR | VTVSS |
| scFv VH | KPGASVK | NO: 299) | MG (SEQ | FRG | YMELSSL | FFDF | (SEQ ID |
| | VSCKASP | | ID NO: | (SEQ ID | RSEDTAV | (SEQ ID | NO: 304) |
| | KGFH | | 300) | NO: 301) | YYCAR | NO: 303) | |
| | (SEQ ID | | | | (SEQ ID | | |
| | NO: 298) | | | | NO: 302) | | |
| Consensus | | GX1X2MH, | | FINPYND | | GNGX1X2 | |
| VHCDR | | | | DIQSNER | | X3DGA | |
| | | wherein | | FRG | | YRFFDF, | |
| | | X1 is Yor | | (SEQ ID | | wherein | |
| | | F, and X2 | | NO: 306) | | X1 is K or | |
| | | is P, H, V, | | | | M, X2 is | |
| | | Y, K, or A | | | | WorN, | |
| | | (SEQ ID | | | | and X3 is | |
| | | NO: 305) | | | | G or F | |
| | | | | | | (SEQ ID | |
| | | | | | | NO: 307) | |

TABLE 8
Exemplary light chain CDRs and FWRs of TRBC2-targeting
antigen binding domains

| Ab ID | VLFWR1 | VLCDR1 | VLFWR2 | VLCDR2 | VLFWR3 | VLCDR3 | VLFWR4 |
|---|---|---|---|---|---|---|---|
| JVD2-VL | DVVMTQ | RSSQNL | WYLQKP | RVSNRF | GVPDRFS | SQSTHV | FGGGTK |
| | SPLSLPV | VHSNGR | GQSPQL | P (SEQ | GSGSGTD | PYT | VEIK |
| | TPGEPAS | TYLH | LIY (SEQ | ID NO: | FTLKISR | (SEQ ID | (SEQ ID |
| | ISC (SEQ | (SEQ ID | ID NO: | 311) | VEAEDV | NO: 313) | NO: 314) |
| | ID NO: | NO: 309) | 310) | | GVYFC | | |
| | 308) | | | | (SEQ ID | | |
| | | | | | NO: 312) | | |
| JVD34-VL | DVVMTQ | RSSQNL | WYLQKP | RVSNRF | GVPDRFS | SQSTHV | FGGGTK |
| | SPLSLPV | VHSNGR | GQSPQL | P (SEQ | GSGSGTD | PYT | VEIK |
| | TPGEPAS | TYLQ | LIY (SEQ | ID NO: | FTLKISR | (SEQ ID | (SEQ ID |
| | ISC (SEQ | (SEQ ID | ID NO: | 318) | VEAEDV | NO: 320) | NO: 321) |
| | ID NO: | NO: 316) | 317) | | GVYFC | | |
| | 315) | | | | (SEQ ID | | |
| | | | | | NO: 319) | | |
| Consensus | | RSSQNL | | RVSNRF | | SQSTHV | |
| VLCDR | | VHSNGR | | P (SEQ | | PYT | |
| | | TYLX, | | ID NO: | | (SEQ ID | |
| | | wherein X | | 323) | | NO: 324) | |
| | | is Q or H | | | | | |
| | | (SEQ ID | | | | | |
| | | NO: 322) | | | | | |

TABLE 9
Exemplary variable regions of TRBC2-targeting antigen binding domains

| SEQ ID NO | Description | Sequence |
|---|---|---|
| SEQ ID | JVD3-VH | QVQLVQSGAEVKKPGASVKVSCKASGYTFPGFVMHWVRQAP |
| NO: 325 | | GQGLEWMGFINPYNDDIQSNERFRGRVTMTSDKSTTTAYMEL |
| | | SSLRSEDTAVYYCARGNGMWFDGAYRFFDFWGQGTLVTVSS |
| SEQ ID | JVD4-VH | QVQLVQSGAEVKKPGASVKVSCKASGYPFHGYVMHWVRQAP |
| NO: 326 | | GQGLEWMGFINPYNDDIQSNERFRGRVTMTSDKSTTTAYMEL |
| | | SSLRSEDTAVYYCARGNGKNFDGAYRFFDFWGQGTLVTVSS |
| SEQ ID | JVD5-VH | QVQLVQSGAEVKKPGASVKVSCKASGYTYPGYVMHWVRQA |
| NO: 327 | | PGQGLEWMGFINPYNDDIQSNERFRGRVTMTSDKSTTTAYME |
| | | LSSLRSEDTAVYYCARGNGKWFDGAYRFFDFWGQGTLVTVSS |
| SEQ ID | JVD6-VH | QVQLVQSGAEVKKPGASVKVSCKASNQNFHGYHMHWVRQA |
| NO: 328 | | PGQGLEWMGFINPYNDDIQSNERFRGRVTMTSDKSTTTAYME |
| | | LSSLRSEDTAVYYCARGNGKWGDGAYRFFDFWGQGTLVTVS |
| | | S |
| SEQ ID | JVD7-VH | QVQLVQSGAEVKKPGASVKVSCKASSQNFHGFYMHWVRQAP |
| NO: 329 | | GQGLEWMGFINPYNDDIQSNERFRGRVTMTSDKSTTTAYMEL |
| | | SSLRSEDTAVYYCARGNGKWGDGAYRFFDFWGQGTLVTVSS |
| SEQ ID | JVD8-VH | QVQLVQSGAEVKKPGASVKVSCKASYQDFHGYKMHWVRQA |
| NO: 330 | | PGQGLEWMGFINPYNDDIQSNERFRGRVTMTSDKSTTTAYME |
| | | LSSLRSEDTAVYYCARGNGKWGDGAYRFFDFWGQGTLVTVS |
| | | S |
| SEQ ID | JVD9-VH | QVQLVQSGAEVKKPGASVKVSCKASGYNFHGFYMHWVRQAP |
| NO: 331 | | GQGLEWMGFINPYNDDIQSNERFRGRVTMTSDKSTTTAYMEL |
| | | SSLRSEDTAVYYCARGNGKWGDGAYRFFDFWGQGTLVTVSS |
| SEQ ID | BKM0097 anti- | QVQLVQSGAEVKKPGASVKVSCKASTSGFHGYPMHWVRQAP |
| NO: 332 | TRBC2 VH; | GQGLEWMGFINPYNDDIQSNERFRGRVTMTSDKSTTTAYMEL |
| | BJM1184 VH | SSLRSEDTAVYYCARGNGKWGDGAYRFFDFWGQGTLVTVSS |
| SEQ ID | BKM0098 anti- | QVQLVQSGAEVKKPGASVKVSCKASPRGFHGYHMHWVRQAP |
| NO: 333 | TRBC2 VH; | GQGLEWMGFINPYNDDIQSNERFRGRVTMTSDKSTTTAYMEL |
| | BJM1185 VH | SSLRSEDTAVYYCARGNGKWGDGAYRFFDFWGQGTLVTVSS |
| SEQ ID | BJM1186 VH | QVQLVQSGAEVKKPGASVKVSCKASFQDFHGYAMHWVRQAP |
| NO: 334 | | GQGLEWMGFINPYNDDIQSNERFRGRVTMTSDKSTTTAYMEL |
| | | SSLRSEDTAVYYCARGNGKWGDGAYRFFDFWGQGTLVTVSS |
| SEQ ID | BJM1187 VH | QVQLVQSGAEVKKPGASVKVSCKASSKDFHGFAMHWVRQAP |
| NO: 335 | | GQGLEWMGFINPYNDDIQSNERFRGRVTMTSDKSTTTAYMEL |
| | | SSLRSEDTAVYYCARGNGKWGDGAYRFFDFWGQGTLVTVSS |
| SEQ ID | BC2_YR3_B11- | QVQLVQSGAEVKKPGASVKVSCKASPKGFHGYHMHWVRQAP |
| NO: 336 | scFv VH | GQGLEWMGFINPYNDDIQSNERFRGRVTMTSDKSTTTAYMEL |
| | | SSLRSEDTAVYYCARGNGKWGDGAYRFFDFWGQGTLVTVSS |
| SEQ ID | JVD2-VL | DVVMTQSPLSLPVTPGEPASISCRSSQNLVHSNGRTYLHWYLQ |
| NO: 337 | | KPGQSPQLLIYRVSNRFPGVPDRFSGSGSGTDFTLKISRVEAED |
| | | VGVYFCSQSTHVPYTFGGGTKVEIK |
| SEQ ID | JVD34-VL | DVVMTQSPLSLPVTPGEPASISCRSSQNLVHSNGRTYLQWYLQ |
| NO: 338 | | KPGQSPQLLIYRVSNRFPGVPDRFSGSGSGTDFTLKISRVEAED |
| | | VGVYFCSQSTHVPYTFGGGTKVEIK |

TABLE 10
Exemplary TRBC2-targeting antigen binding domains/antibody molecules

| SEQ ID NO | Description | Sequence |
|---|---|---|
| SEQ ID | BC2_YR3-A12- | QVQLVQSGAEVKKPGASVKVSCKASTSGFHGYPMHWVRQA |
| NO: 339 | scFv (BJM1184) | PGQGLEWMGFINPYNDDIQSNERFRGRVTMTSDKSTTTAYME |
| | | LSSLRSEDTAVYYCARGNGKWGDGAYRFFDFWGQGTLVTVS |
| | | SGGGGSGGGGSGGGGSGGGGSDVVMTQSPLSLPVTPGEPASI |
| | | SCRSSQNLVHSNGRTYLQWYLQKPGQSPQLLIYRVSNRFPGV |
| | | PDRFSGSGSGTDFTLKISRVEAEDVGVYFCSQSTHVPYTFGGG |
| | | TKVEIK |
| SEQ ID | BC2_YR3-A5- | QVQLVQSGAEVKKPGASVKVSCKASPRGFHGYHMHWVRQA |
| NO: 340 | scFv (BJM1185) | PGQGLEWMGFINPYNDDIQSNERFRGRVTMTSDKSTTTAYME |
| | | LSSLRSEDTAVYYCARGNGKWGDGAYRFFDFWGQGTLVTVS |
| | | SGGGGSGGGGSGGGGSGGGGSDVVMTQSPLSLPVTPGEPASI |
| | | SCRSSQNLVHSNGRTYLQWYLQKPGQSPQLLIYRVSNRFPGV |
| | | PDRFSGSGSGTDFTLKISRVEAEDVGVYFCSQSTHVPYTFGGG |
| | | TKVEIK |
| SEQ ID | BC2_YR3-B3- | QVQLVQSGAEVKKPGASVKVSCKASFQDFHGYAMHWVRQA |
| NO: 341 | scFv (BJM1186) | PGQGLEWMGFINPYNDDIQSNERFRGRVTMTSDKSTTTAYME |
| | | LSSLRSEDTAVYYCARGNGKWGDGAYRFFDFWGQGTLVTVS |
| | | SGGGGSGGGGSGGGGSGGGGSDVVMTQSPLSLPVTPGEPASI |
| | | SCRSSQNLVHSNGRTYLQWYLQKPGQSPQLLIYRVSNRFPGV |
| | | PDRFSGSGSGTDFTLKISRVEAEDVGVYFCSQSTHVPYTFGGG |
| | | TKVEIK |
| SEQ ID | BC2_YR3_B4- | QVQLVQSGAEVKKPGASVKVSCKASSKDFHGFAMHWVRQA |
| NO: 342 | scFv (BJM1187) | PGQGLEWMGFINPYNDDIQSNERFRGRVTMTSDKSTTTAYME |
| | | LSSLRSEDTAVYYCARGNGKWGDGAYRFFDFWGQGTLVTVS |
| | | SGGGGSGGGGSGGGGSGGGGSDVVMTQSPLSLPVTPGEPASI |
| | | SCRSSQNLVHSNGRTYLQWYLQKPGQSPQLLIYRVSNRFPGV |
| | | PDRFSGSGSGTDFTLKISRVEAEDVGVYFCSQSTHVPYTFGGG |
| | | TKVEIK |
| SEQ ID | BC2_YR3B_11- | QVQLVQSGAEVKKPGASVKVSCKASPKGFHGYHMHWVRQA |
| NO: 343 | scFv | PGQGLEWMGFINPYNDDIQSNERFRGRVTMTSDKSTTTAYME |
| | | LSSLRSEDTAVYYCARGNGKWGDGAYRFFDFWGQGTLVTVS |
| | | SGGGGSGGGGSGGGGSGGGGSDVVMTQSPLSLPVTPGEPASI |
| | | SCRSSQNLVHSNGRTYLQWYLQKPGQSPQLLIYRVSNRFPGV |
| | | PDRFSGSGSGTDFTLKISRVEAEDVGVYFCSQSTHVPYTFGGG |
| | | TKVEIK |

### Chimeric Antigen Receptors

A chimeric antigen receptor comprises: (a) an extracellular antigen binding domain, (b) a transmembrane domain and (c) at least one intracellular domain.

In one embodiment, said antigen is a TRBC protein. In one embodiment, said TRBC protein is TRBC1 protein. In one embodiment, said TRBC protein is TRBC2 protein. In one embodiments, said antigen domain comprises a TRBC binding domain described herein.

In one embodiment, said antigen binding domain is an antibody, an ScFv, a bispecific antibody, a diabody, a tribody, a tetrabody, or a functional fragment or variant thereof.

In some embodiments, said antigen binding domain is connected to a single transmembrane domain either directly or via one or more peptide linkers. In some embodiments, the linker is a hinge region of a protein. In some embodiments, the extracellular antigen binding domain is directly fused to the transmembrane domain. The transmembrane domain spans the membrane bridging the extracellular and intracellular sections of the CAR. The transmembrane domain is linked at the cytosolic end with one or more intracellular domain, wherein at least one of the intracellular domains is capable of signaling via an intracellular signaling pathway that is associated with the activation of the CAR-expressing cell, for example the NK-CAR cell or CAR T cell. The one or more intracellular domains may be connected to each other via a linker. The extracellular antigen binding domain, the transmembrane domain and the one or more intracellular domains are operably linked with each other so that upon binding of the extracellular antigen binding domain of the CAR to its cognate antigen, the intracellular signaling domain is activated to send/receive intracellular signaling for the activation of the NK cell or T cell.

In some embodiments, a linker is a short peptide that structurally links two peptide domains. The linkers may be comprised of 1, 2, 3, 4, 5, 6, 7, 8, 9 or 10 amino acids, or longer. In some embodiments, the linker may provide structural modularity, but may be otherwise biologically inert. In some embodiments, the amino acids in a linker may be comprised of alanine, serine and/or glycine. In some embodiments, the amino acids in a linker may be comprised of serine and glycine. In some embodiments, the linker is composed of a string of glycine and serine repeats. In some embodiments, the linker is composed of a single amino acid repeat, or a dimer, or a dimer repeated in tandem. An exemplary linker may have an amino acid sequence: GGGGS (SEQ ID NO: 213). Another exemplary linker may have an amino sequence GGGSSGS (SEQ II) NO 214). Another exemplary linker may have an amino sequence: SGSSGGSSSGA (SEQ ID NO: 215). In some embodiments, the linker is flexible in some embodiments, the linker is rigid.

### Extracellular Antigen Binding Domain

The antigen binding domain selectively binds to a target antigen expressed on the surface of a cell. An antigen binding domain can be any protein or peptide that selectivity binds to the target antigen (e.g., TRBC1 or TRBC2), e.g., a ligand, a receptor or a functional fragment thereof, or an antibody or a functional fragment or variant thereof. In some embodiments, antigen binding domain is a single chain variable domain (scFv). ScFVs comprise a heavy chain variable region (VH) a light chain variable region (VL) linked by a peptide.

In some embodiments, the extracellular domain comprises a TRBC1 binding domain. In some embodiments, the extracellular domain comprises a TRBC2 binding domain. In some embodiments, the extracellular domain comprises a TRCB1 binding scFv. In some embodiment, the extracellular domain comprises a TRCB2 binding scFv.

In some embodiments, the extracellular domain comprises a TRCB1 binding scFv that comprises a heavy chain variable region (VH) comprising a variable heavy chain VHCDR1, VHCDR2, VHCDR3 interspersed with framework regions VHFWR1, VHFWR2, VHFWR3 and VHFWR4; and a light chain variable region (VL) comprising a variable heavy chain VLCDR1, VLCDR2, VLCDR3 interspersed with framework regions VLFWR1, VLFWR2, VLFWR3 and VLFWR4; where the heavy chain and the light chain are interconnected by a short peptide linker (e.g., a flexible linker).

In some embodiments, the extracellular domain comprises a TRCB2 binding scFv that comprises a heavy chain variable region (VH) comprising a variable heavy chain VHCDR1, VHCDR2, VHCDR3 interspersed with framework regions VHFWR1, VHFWR2, VHFWR3 and VHFWR4; and a light chain variable region (VL) comprising a variable heavy chain VLCDR1, VLCDR2, VLCDR3 interspersed with framework regions VLFWR1, VLFWR2, VLFWR3 and VLFWR4; where the heavy chain and the light chain are interconnected by a short peptide linker (e.g., a flexible linker).

In some embodiments, the VH and the VL are arranged in a linear sequence as follows: VHFWR1-VHCDR1-VHFWR2-VHCDR2-VHFWR3-VHCDR3-VHFWR4-linker-VLWFR1-VLCDR1-VLFWR2-VLCDR2-VLFWR3-VLCDR3-VLWFR4.

In some embodiments, the antigen binding domain that selectively targets lymphocytes expressing a T cell receptor comprising TRBC1 or TRBC2 comprises any CDR amino acid sequence, framework region (FWR) amino acid sequence, or variable region amino acid sequence disclosed in Tables 1-10. In some embodiments, the antigen binding domain that selectively targets lymphocytes expressing a T cell receptor comprising TRBC1 or TRBC2 comprises heavy and/or light chain amino acid sequences of Table 5.

In some embodiments, the TRBC antigen binding domain comprises a heavy chain and a light chain, wherein: the heavy chain comprising an amino acid sequence of SEQ ID NO: 176, and the light comprising an amino acid sequence of SEQ ID NO: 186; or, the heavy chain comprising an amino acid sequence of SEQ ID NO: 203, and the light comprising an amino acid sequence of SEQ ID NO: 204; or, the heavy chain comprising an amino acid sequence of SEQ ID NO: 205, and said light comprising an amino acid sequence of SEQ ID NO: 206; or, the heavy chain comprising an amino acid sequence of SEQ ID NO: 207, and the light comprise an amino acid sequence of SEQ ID NO: 208; or, the heavy chain comprising an amino acid sequence of SEQ ID NO: 209, and the light comprising an amino acid sequence of SEQ ID NO: 210; or, the heavy chain comprising an amino acid sequence of SEQ ID NO: 211, and the light comprising an amino acid sequence of SEQ ID NO: 212.

In some embodiments, the TRBC antigen binding domain comprises a heavy chain and a light chain, wherein the heavy chain comprising an amino acid sequence of SEQ ID NO: 325, and the light comprising an amino acid sequence of SEQ ID NO: 337 or 338; or, the heavy chain comprising an amino acid sequence of SEQ ID NO: 326, and the light comprising an amino acid sequence of SEQ ID NO: 337 or 338; or, the heavy chain comprising an amino acid sequence of SEQ ID NO: 327, and said light comprising an amino acid sequence of SEQ ID NO: 337 or 338; the heavy chain comprising an amino acid sequence of SEQ ID NO: 327, and the light comprising an amino acid sequence of SEQ ID NO: 337 or 338; or, the heavy chain comprising an amino acid sequence of SEQ ID NO: 329, and the light comprising an amino acid sequence of SEQ ID NO: 337 or 338; or, the heavy chain comprising an amino acid sequence of SEQ ID NO: 330, and said light comprising an amino acid sequence of SEQ ID NO: 337 or 338; the heavy chain comprising an amino acid sequence of SEQ ID NO: 331, and the light comprising an amino acid sequence of SEQ ID NO: 337 or 338; or, the heavy chain comprising an amino acid sequence of SEQ ID NO: 332, and the light comprising an amino acid sequence of SEQ ID NO: 337 or 338; or the heavy chain comprising an amino acid sequence of SEQ ID NO: 333, and the light comprising an amino acid sequence of SEQ ID NO: 337 or 338; or, the heavy chain comprising an amino acid sequence of SEQ ID NO: 334, and said light comprising an amino acid sequence of SEQ ID NO: 337 or 338; or, the heavy chain comprising an amino acid sequence of SEQ ID NO: 335, and the light comprise an amino acid sequence of SEQ ID NO: 337 or 338.

In some embodiments, the TRBC antigen binding domain comprises: (a) a heavy chain comprising a (i) complementarity determinant region (CDR)1 (CDR1); (ii) a CDR2: (iii) a CDR3; (iv) a framework region 1 (FWR1), (v) a FWR2, (vi) FWR3, and (vii) a FDR4, and (b) a light chain comprising a (i) complementarity determinant region (CDR)1 (CDR1); (ii) a CDR2: (iii) a CDR3; (iv) a framework region 1 (FWR1), (v) a FWR2, (vi) FWR3, and (vii) a FDR4; having said corresponding amino acid sequences listed in Tables 1-3.

In some embodiments, said TRBC antigen binding domain comprises a heavy chain and a light chain, wherein: (i) said heavy chain comprises an amino acid sequence from any one of SEQ ID NOs: 176, 177, 178, 179, 180, 181, 182, 183, 184, and 185; and (ii) said light chain comprises an amino acid sequence from any one of said SEQ ID NOs: 186, 187, 188, 189, 190, 191, 192, 193, 194, and 195.

In some embodiments, said TRBC antigen binding domain comprises a heavy chain and a light chain, wherein: (i) said heavy chain comprises an amino acid sequence from any one of SEQ ID NOs: 176, or a sequence having at least 90% amino acid sequence identity to SEQ ID NO: 176; and (ii) said light chain comprises an amino acid sequence from any one of SEQ ID NOs: 186, or a sequence having at least 90% amino acid sequence identity to SEQ ID NO: 186.

In some embodiments, the TRBC antigen binding domain comprises an scFV having a heavy chain and a light chain, wherein the heavy chain comprising an amino acid sequence of SEQ ID NO: 176, and the light comprising an amino acid sequence of SEQ ID NO: 186.

In some embodiments, the TRBC antigen binding domain comprises an scFV having a heavy chain comprising an amino acid sequence of SEQ ID NO: 203, and a light comprising an amino acid sequence of SEQ ID NO: 204.

In some embodiments, the TRBC antigen binding domain comprises an scFV having a heavy chain comprising an amino acid sequence of SEQ ID NO: 205, and a light comprising an amino acid sequence of SEQ ID NO: 206.

In some embodiments, the TRBC antigen binding domain comprises an scFV having a heavy chain comprising an amino acid sequence of SEQ ID NO: 207, and the light comprising an amino acid sequence of SEQ ID NO: 208.

In some embodiments, the TRBC antigen binding domain comprises an scFV having a heavy chain comprising an amino acid sequence of SEQ ID NO: 209, and the light comprising an amino acid sequence of SEQ ID NO: 210.

In some embodiments, the TRBC antigen binding domain comprises an scFV having a heavy chain comprising an amino acid sequence of SEQ ID NO: 211, and the light comprising an amino acid sequence of SEQ ID NO: 212.

In some embodiments, the TRBC antigen binding domain wherein said TRBC antigen binding domain comprises: (a) a heavy chain comprising a (i) complementarity determinant region (CDR)1 (CDR1); (ii) a CDR2: (iii) a CDR3; (iv) a framework region 1 (FWR1), (v) a FWR2, (vi) FWR3, and (vii) a FDR4, and (b) a light chain comprising a (i) complementarity determinant region (CDR)1 (CDR1); (ii) a CDR2: (iii) a CDR3; (iv) a framework region 1 (FWR1), (v) a FWR2, (vi) FWR3, and (vii) a FDR4; having the corresponding amino acid sequences listed in Tables 1-3.

### Transmembrane Domain

In some embodiments the TM domain comprises a CD8a transmembrane domain. In some embodiments, the CD8a transmembrane domain is derived from a CD8a transmembrane domain having the amino acid sequence of the spacer transmembrane region is preferably selected from amino acids 118-210 of CD8a or from amino acids 128-210. For example, the amino acid sequence of the spacer transmembrane region is as shown in any one of the following amino acid sequence groups: amino acids 118-210 of CD8a, amino acids 119-210, amino acids 120-210, 121-210 amino acids, amino acids 122-210, amino acids 123-210, amino acids 124-210, amino acids 125-210, amino acids 126-210, amino acids 127-210, or 128-210 amino acids. In some embodiments the TM domain also comprises a CD8a hinge region.

In some embodiments, the TM domain comprises a CD28 TM domain. In some the TM comprises a CD68 binding domain. In some embodiments, the TM binding domain is a CD19 domain. In some embodiments the TM domain is a derived from a NKG2D binding receptor protein transmembrane domain.

In some embodiments, the recombinant CAR is generated for expression in a T cell to generate a CART cell, that has a binding specificity to TRBC1 or TRBC2, a transmembrane domain and an intracellular signaling domain, capable of intracellular signaling for T cell activation. In some embodiments, the transmembrane domain comprises a transmembrane domain of a protein chosen from the alpha, beta or zeta chain of T-cell receptor, CD28, CD3 epsilon, CD45, CD4, CD5, CD8, CD9, CD16, CD22, CD33, CD37, CD64, CD80, CD86, CD134, CD137 and CD154. In some embodiments, the transmembrane domain comprises a transmembrane domain of CD8.

CD8 hinge domain may comprises an amino acid sequence:

(SEQ ID NO: 216)
TTTPAPRPPTPAPTIASQPLSLRPEACRPAAGGAVHTRGLDFACD

CD8 transmembrane region may comprise an amino acid sequence:

(SEQ ID NO: 217)
IYTWAPLAGTCGVLLLSLVITLYC

### Intracellular Domain

In some embodiments the CAR for NK or T cells comprises an intracellular domain having a intracellular signaling region. The intracellular signaling domain signals activation of the NK or T cells. In some embodiments the CAR comprises one or more intracellular domains. In some embodiments the CAR comprises an intracellular signaling domain and an signaling protein recruitment domain. In some embodiments the intracellular signaling domain comprises a phosphorylating domain. In some embodiments the intracellular signaling domain comprises a phosphorylating domain. In some embodiments the intracellular signaling domain comprises a recruitment for an intracellular domain having a phosphorylating domain. In some embodiments the intracellular domain is derived from a DAP12 signaling domain. In some embodiments an intracellular signaling domain is derived from any one of CD2, CD48, CD58, signaling lymphocytic activation molecule (SLAM), (CD150), 2B4 (CD244), CD84, Ly-9 (CD229), NK-T-B-Ag (NTB-A), CD2-like receptor-activating cytotoxic cell (CRACC), and B lymphocyte activator macrophage expressed (BLAME) protein intracellular domains. In some embodiments, the intracellular domain is derived from the cytoplasmic domains of SLAM, 2B4, CD84, Ly-9, CRACC, or NTB-A, which may contain unique amino acid motifs, TxYxxV/I, called immunoreceptor tyrosine-based switch motifs (ITSMs). ITSMs are associate with a Src homology 2 (SH2) domain-containing protein, SLAM-associated protein (SAP; also called SH2D1A or DSHP), predominantly expressed in T and NK cells. SAP recruits and activates the Src-family kinase Fyn through a unique SH2-SH3 domains interaction. Fyn induces phosphorylation of the cytoplasmic domain of ITSM-containing receptors, allowing sequential recruitment and activation of downstream signaling adaptors and effectors such as SHIP-1, Shc, Dok1/2, and Ras-GAP. Moreover, SAP can function as a blocker, inhibiting the recruitment of protein tyrosine phosphatases like Src homology-2 phosphotyrosine phosphatase (SHP-2) to the cytoplasmic domain of ITSM-containing receptors. Any of the receptor signaling moieties can be incorporated as a functional intracellular domain of the CAR directed to be expressed in the NK cells, and may assist in activating NK cells upon engagement of the extracellular domain with the target antigen.

In some embodiments the CAR is designed for activation upon binding to a cancer cell, such as a T cell that expresses TRB protein. The CAR binds to a TRB protein with the TRB antigen binding extracellular domain and is activated to transduce the signal to the intracellular domain of the CAR, wherein the intracellular domain is activated and initiates signaling crosstalk within the cytoplasm and nucleus of the NK cell, thereby activating the NK cell, and augmenting its killing function. The NK cell cytotoxicity is thereafter directed to the cancer cell to which it is bound.

In some embodiments, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule. A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, FcR gamma, FcR beta, CD3 gamma, CD3 delta, CD3 epsilon, CD5, CD22, CD79a, CD79b, CD278 (also known as “ICOS”), FccRI, CD66d, DAP10 and DAP12.

In some embodiments, the cytoplasmic domain of zeta comprises residues 52 through 164 of GenBank Acc. No. BAG36664.1 or a variant thereof (for example, a molecule having mutations, for example, point mutations, fragments, insertions, or deletions). In some embodiments, the “zeta stimulatory domain” or a “CD3-zeta stimulatory domain” may comprise an amino acid sequence as:

(SEQ ID NO: 218)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR.

In some embodiments, the costimulatory intracellular signaling domain of a CAR for expressing in a T cell is a 4-1BB (CD137) intracellular domain having an amino acid sequence: KRGRKKLLYIFKQPFMRPVQTTQEEDGCSCRFPEEEEGGCEL (SEQ ID NO: 219). In some embodiments, the intracellular signaling domain of a CAR for expressing in a T cell is a CD28 intracellular domain having an amino acid sequence: RSKRSRLLHSDYMNMTPRRPGPTRKHY QPYAPPRDFAAYRS (SEQ ID NO: 220).

In one aspect, provide herein is a vector, e.g., an expression vector, comprising a nucleic acid molecule disclosed herein.

In some embodiments, the encoded chimeric antigen receptor (CAR) is expressed in frame and as a single polypeptide chain.

Provided herein are, inter alia, vectors comprising the nucleic acid molecule described herein. In some embodiments, the vector is a DNA vector, a RNA vector, a plasmid, a lentivirus vector, an adenoviral vector, or a retrovirus vector.

### NK Cells

In one aspect, provided herein is a therapeutically effective NK cell for adoptive immunotherapy. In some embodiments, the NK cell is effective for cancer immunotherapy. The NK cell for adoptive immunotherapy incorporates a recombinant nucleic acid molecule that encodes a chimeric antigen receptor (CAR); and the NK cell expresses said CAR. In some embodiments, the CAR comprises an anti-TRBC antigen binding domain.

In some embodiments, a chimeric antigen receptor described herein (e.g., a chimeric antigen receptor that comprises an anti-TRBC antigen binding domain) is expressed by an NK cell. In some embodiments, a population of NK cells are engineered ex vivo to express a CAR described herein to produce a population of NK-CAR cells.

Provided herein is a method wherein NK cells are modified ex vivo by introducing into the NK cell, a recombinant nucleic acid encoding a chimeric antigen protein, thereby obtaining a NK-CAR, whereupon the NK-CAR cell is used for adoptive transfer for cancer therapy.

### Population of NK Cells

In some embodiments, a population of NK cells that is engineered ex vivo to express a CAR described herein is obtained from a subject, wherein the subject is human. In some embodiments, the population of NK cells is obtained from a peripheral blood draw, or from apheresis or leukapheresis sample from a human subject. In some embodiments the population of NK cells are concentrated from more than one biological sample obtained from one human subject.

In some embodiments the biological sample is peripheral blood. In some embodiments the biological sample is a leukapheresis pack. In some embodiments, the NK cells are concentrated from 1, 2, 3, 4, 5, or more peripheral blood samples to obtain a therapeutically effective number of NK cells in the population of NK cells for preparing NK-CAR cells for an adoptive transfer. In some embodiments, the NK cells are concentrated from 1, 2, 3, 4, 5, or more leukapheresis packs in order to obtain a therapeutically effective number of NK cells in the population of NK cells for preparing NK-CAR cells for an adoptive transfer.

### NK-CAR Cells

NK-CAR cells are derived from the population of NK cells withdrawn from or derived from a biological sample, which are further modified by engineering ex vivo. In some embodiments the NK cells are modified or engineered by incorporating within the population of NK cells a recombinant nucleic acid comprising a nucleic acid sequence encoding a CAR. In some embodiments, the incorporating comprises contacting the population of NK cells a recombinant nucleic acid comprising a nucleic acid sequence encoding a CAR. In some embodiments, the incorporating comprises electroporating the population of NK cells a recombinant nucleic acid comprising a nucleic acid sequence encoding a CAR. In some embodiments, the incorporating comprises transfecting the population of NK cells a recombinant nucleic acid comprising a nucleic acid sequence encoding a CAR. The recombinant nucleic acid molecule comprising a nucleic acid sequence encoding a CAR may be comprised in a vector. A vector is a DNA vector. In addition to the nucleic acid sequence encoding a CAR the vector may comprise one or more additional sequences, such as a promoter, and enhancer, a 5′UTR, a 3′UTR, a poly A sequence, a localization sequence, a leader sequence or one or more further regulatory sequence. Exemplary promoter is a CMV promoter. An exemplary 3′ or 5′ UTR may be derived from a GCSF, GMCSF, beta actin, globulin or WPRE gene sequences, as is known to one of skill in the art. Alternatively, the recombinant nucleic acid comprising a nucleic acid sequence encoding a CAR may be inside a lentiviral, an adenoviral or a retroviral transduction system or cassette and is transduced into the population of NK cells. In alternative embodiments, the recombinant nucleic acid molecule is a RNA molecule. An RNA molecule may be incorporated into a population of NK cells by electroporation or by chemical transfection, such as in composition comprising the nucleic acid and one or more lipid components, as is known to one of skill in the art.

Upon contacting the population of NK cells with the recombinant nucleic acid comprising a nucleic acid sequence encoding a CAR via transfection or transduction or via any other known means of incorporation of a nucleic acid molecule into a cell, the recombinant nucleic acid may be incorporated in at least 50%, at least 60%, at least 70%, at least 80%, at least 90%, at least 92%, at least 95%, or at least 100% cells of the population of NK cells. Accordingly, at least 50%, at least 60%, at least 70%, at least 80%, at least 90%, at least 92%, at least 95%, or at least 100% cells of the population of NK cells may express CAR on their surface which can be determined in about 24 hours by a flow cytometric assay known to one of skill in the art. Accordingly, at least 50%, at least 60%, at least 70%, at least 80%, at least 90%, at least 92%, at least 95%, or at least 100% cells of the population of NK cells that express CAR on their surface are NK-CAR cells.

NK CAR cells express CAR. NK-CAR cells exhibit binding to a target cell, for example a cell expressing a target protein, for example a cell expressing a TRBC1 protein to which the CAR extracellular domain is designed to bind to, as described in the previous sections. An NK-CAR cells is an activated NK cell. An NK-CAR cell secretes TNF-alpha in the presence of a target cell. TNF-alpha secretion may be increased at least 2-fold, at least 3-fold, at least 4-fold, at least 5-fold, at least 10-fold, at least 50-fold at least 100-fold or at least 1000-fold compared to a non-activated NK cell of a population of NK cells that does not express CAR. An NK-CAR cell secretes IFN-gamma in the presence of a target cell. IFN-gamma secretion may be increased at least 2-fold, at least 3-fold, at least 4-fold, at least 5-fold, at least 10-fold, at least 50-fold at least 100-fold or at least 1000-fold compared to a non-activated NK cell of a population of NK cells that does not express CAR.

An NK-CAR cell may secrete one or more cytokines or chemokines from any one of, IL-1b, IL-6, IL-7, IL-10, IL-12p40, IFN-alpha, MIP-1alpha, MIP-1beta, RANTES and CXCL9 in presence of a target cell, that is higher than an NK cell not expressing a CAR. An NK-CAR cell exhibits higher cytotoxicity than an NK cell not expressing CAR towards a target cell, such as a target cancer cell expressing TRBC1.

An NK-CAR cell may be tested for any one or more of cell surface protein expression assay (such as by flow cytometry or western blot assay), cytokine release assay (such as by flow cytometry or ELISA), cytotoxicity assay by any known cytotoxicity assay formats known to one of skill in the art before preparing the NK-CAR for therapeutic purpose, for example, preparing into a pharmaceutical composition.

In some embodiments, the population of NK-CAR cells are administered to the same subject they were obtained from (e.g., autologous NK-CAR cells).

In some embodiments, the population of NK cells are administered to a different subject than they were obtained from (e.g., allogenic NK-CAR cells). In some cases, the allogeneic NK-CAR cells are further modified to reduce or obliterate immune response mediated destruction of the NK cells when administered into a subject.

### T Cells and CAR T Cells

In one aspect, provided herein is a therapeutically effective T cell for adoptive immunotherapy. In some embodiments, the T cell is effective for cancer immunotherapy. In some embodiments, the T cell for adoptive immunotherapy incorporates a recombinant nucleic acid molecule that encodes a chimeric antigen receptor (CAR); and the T cell expresses said CAR. In some embodiments, the CAR comprises an anti-TRBC antigen binding domain.

In some embodiments, a chimeric antigen receptor described herein (e.g., a chimeric antigen receptor that comprises an anti-TRBC antigen binding domain) is expressed by an T cell. In some embodiments, a population of T cells are engineered ex vivo to express a CAR described herein to produce a population of CAR T cells expressing a CAR having anti-TRBC antigen binding domain.

Provided herein is a method wherein T cells are modified ex vivo by introducing into the T cell, a recombinant nucleic acid encoding a chimeric antigen protein, thereby obtaining a CAR T cell, whereupon the CAR T cell is used for adoptive transfer for cancer therapy. In some embodiments, the CAR T cell is CD8+. In some embodiments, the CAR T cell is CD4+. In some embodiments, the CAR T cells are CD3+. In some embodiments, the CAR T cells are generated from a population of T cells that are derived from a biological sample, such as a PBMC sample or a leukapheresis sample, and are engineered ex vivo to express a CAR as described in the disclosure.

In some embodiments, a population of T cells that is engineered ex vivo to express a CAR described herein is obtained from a subject, wherein the subject is human. In some embodiments, the population of T cells is obtained from a peripheral blood draw, or from apheresis or leukapheresis sample from a human subject.

In some embodiments, T cells derived from a biological sample are contacted with a recombinant nucleic acid molecule that encodes a CAR that comprises an anti-TRBC antigen binding domain to obtain a population of T cells. The recombinant nucleic acid molecule that encodes a CAR is incorporated into a population of T cells via any known methods of nucleic acid incorporation. In some embodiments, the recombinant nucleic acid molecule that encodes a CAR is incorporated into a population of T cells by electroporation. In some embodiments, the recombinant nucleic acid molecule that encodes a CAR is incorporated into a population of T cells by nucleofection. In some embodiments, the recombinant nucleic acid molecule that encodes a CAR is incorporated into a population of T cells by viral transduction, such as a lentiviral or an adenoviral transduction. In some embodiments, the recombinant nucleic acid molecule that encodes a CAR is incorporated into a population of T cells by a plasmid vector. In some embodiments, the recombinant nucleic acid is DNA. In some embodiments, the recombinant nucleic acid is mRNA. In some embodiments, recombinant nucleic acid is encapsulated in a lipid or liposome vector.

In some embodiments, upon contacting the cells in the biological sample with the recombinant nucleic acid molecule, the recombinant nucleic acid may be incorporated in at least 50%, at least 60%, at least 70%, at least 80%, at least 90%, at least 92%, at least 95%, or at least 100% cells of the population of T cells. Accordingly, at least 50%, at least 60%, at least 70%, at least 80%, at least 90%, at least 92%, at least 95%, or at least 100% cells of the population of T cells may express CAR on their surface which can be determined in about 24 hours by a flow cytometric assay known to one of skill in the art. Accordingly, at least 50%, at least 60%, at least 70%, at least 80%, at least 90%, at least 92%, at least 95%, or at least 100% cells of the population of T cells that express CAR on their surface are CAR T cells.

### Methods of Making NK-CAR Cells

In some embodiments, the population of NK cells are electroporated, transfected or transduced with a vector or a nucleic acid molecule comprising a nucleic acid sequence encoding a CAR described herein. In some embodiment, a second, a third, a fourth or a fifth nucleic acid is also introduced into the NK cells to activate the NK cells, and/or to stabilize the NK cells, and/or to protect the NK cells from inhibitory signaling. In some embodiments, the NK cells are further transfected or transduced with one, two, three, four, five, six, seven, eight, nine, ten or more nucleic acids, each expressing a different protein or fragment thereof in the NK cell.

Provided herein are, inter alia, methods of making an NK cell, comprising transducing, transfecting or electroporating the NK cell with a vector described herein.

In some embodiments, the population of NK cells are subjected to a transfection with a recombinant nucleic acid molecule comprising a nucleic acid sequence encoding a CAR in vitro.

In some embodiments, the population of NK cells are subjected to a transduction with a recombinant nucleic acid molecule comprising a nucleic acid sequence encoding a CAR in vitro.

In some embodiments, transduction comprises transduction with a lentiviral vector, the vector comprising the nucleic acid molecule comprising a nucleic acid sequence encoding a CAR.

In some embodiments, the transduction comprises transduction with a lentiviral vector, the vector comprising the nucleic acid molecule comprising a nucleic acid sequence encoding a CAR.

In some embodiments, the transduction comprises transduction with an adenoviral vector, the vector comprising the nucleic acid molecule comprising a nucleic acid sequence encoding a CAR.

In some embodiments, the transduction comprises transduction with an adeno-associated viral (AAV) vector, the vector comprising the nucleic acid molecule comprising a nucleic acid sequence encoding a CAR.

In some embodiments, the population of NK cells are transfected with a vector comprising the recombinant nucleic acid comprising a nucleic acid sequence encoding a CAR. In some embodiments, the population of NK cells are electroporated with a recombinant nucleic acid comprising a nucleic acid sequence encoding a CAR.

The recombinant nucleic acid molecule comprising a nucleic acid sequence encoding a CAR may be comprised in a vector. A vector may be a DNA vector. In addition to the nucleic acid sequence encoding a CAR the vector may comprise one or more additional sequences, such as a promoter, and enhancer, a 5′UTR, a 3′UTR, a poly A sequence, a localization sequence, a leader sequence or one or more further regulatory sequence. Exemplary promoter is a CMV promoter. An exemplary 3′ or 5′ UTR may be derived from a GCSF, GMCSF, beta actin, globulin or WPRE gene sequences, as is known to one of skill in the art.

In some embodiments, the recombinant nucleic acid molecule comprising a nucleic acid sequence encoding a CAR may comprise naked polynucleic acid. In some embodiments, the recombinant nucleic acid molecule comprising a nucleic acid sequence encoding a CAR is DNA. the recombinant nucleic acid molecule comprising a nucleic acid sequence encoding a CAR is RNA. the recombinant nucleic acid molecule comprising a nucleic acid sequence encoding a CAR is messenger RNA (mRNA).

Upon successful incorporation of the recombinant nucleic acid molecule comprising a nucleic acid sequence encoding a CAR in a population of NK cells, at least 50%, at least 60%, at least 70%, at least 80%, at least 90%, at least 92%, at least 95%, or at least 100% cells of the population of NK cells. Accordingly, at least 50%, at least 60%, at least 70%, at least 80%, at least 90%, at least 92%, at least 95%, or at least 100% cells of the population of NK cells may express CAR on their surface which can be determined in about 24 hours by a flow cytometric assay known to one of skill in the art. Accordingly, at least 50%, at least 60%, at least 70%, at least 80%, at least 90%, at least 92%, at least 95%, or at least 100% cells of the population of NK cells that express CAR on their surface are NK-CAR cells. NK CAR cells may be further selected and or enriched for the expression of CARs, for enhanced cytotoxicity profile, for enhanced cytokine secretion profile or a combination thereof.

In one aspect, provide herein is an NK cell comprising a nucleic acid molecule disclosed herein or a vector disclosed herein.

In one aspect, provide herein is a pharmaceutical composition comprising a CAR molecule disclosed herein or an antibody molecule disclosed herein and a pharmaceutically acceptable carrier, excipient, or stabilizer.

In some embodiments, the NK cells are generated by ex vivo incorporation of vector or a nucleic acid molecule encoding the CAR described in any of the embodiments described herein, stabilizing the NK cells in vitro comprising culturing the NK cells comprising the nucleic acid encoding the CAR, in a medium comprising buffer, one or more growth factors, one or more cytokines, one or more chemokines one or more proteins, bioactive peptides or a combination thereof, for at least 1 hour, at least 3 hours, at least 6 hours, at least 10 hours, at least 12 hours, at least about 24 hours. In some embodiments, the CAR expressing NK cells are enriched from the population of NK cells that are exposed, electroporated or otherwise transfected, or transduced with a nucleic acid molecule encoding the CAR. In some embodiments, the NK cells expressing the CAR are enriched by sorting, for example in a flow cytometer. In some embodiments, the NK-CAR cells are enriched via positive selection sorting; in some embodiments, for example, alternatively the NK-CAR cells are selected by means of negative selection sorting, or a combination thereof. One of skill in the art is aware of positive and negative selection methods by FACS. In some embodiments, the NK cells expressing the CAR are enriched for live cells, expression of CAR, for identification of one or more cell surface markers, including but not limited to activation markers, cytokine or chemokine production or a combination thereof. In some embodiments, an activation markers for a NK-CAR cell comprises induction of TNF-alpha. In some embodiments, an activation markers for a NK-CAR cell comprises induction of IFN-gamma. In some embodiments, an activation marker for a NK-CAR cell may comprise one or more of TNF-alpha, IFN-gamma, IL-1b, IL-6, IL-7, IL-10, IL-12p40, IFN-alpha, MIP-1alpha, MIP-1beta, RANTES and CXCL9.

In some embodiments, the NK cell composition for infusing (NK-CAR cell composition) comprises at least 98%, NK cells that are live cells by a live dead cell assay.

The method of selecting NK-CAR cells for a pharmaceutical composition comprises selecting live NK cells exhibiting positive CAR expression, for positive activation marker expression, and/or for cytokine expression (such as at least one of TNF-alpha and IFN-gamma expression) and/or for cytotoxicity, determined by a standard cytotoxicity assay.

In some embodiments, the cells are not sorted. In some embodiments an aliquot of the sample NK-CAR cells are tested for any or all of the parameters described above.

In some embodiments, the NK cell composition for infusing (NK-CAR cell composition) comprises at least 70%, at least 80%, at least 90% or at least 95% cells that are cytotoxic to a cell expressing the TRBC on the cell surface in vitro.

Following incorporation of a nucleic acid molecule comprising a nucleic acid sequence encoding a CAR in a population of NK cells, and incubating the cells in a media for about 1 to about 24 hours in vitro, the cells may be stored in liquid nitrogen (−70° C.) for future use. Alternatively, in some embodiments, NK cells may be stored in liquid nitrogen (−70° C.) for future use without delay following incorporation of said nucleic acid sequence encoding a CAR. In yet another embodiment, NK cells may be sorted following incubation in a media in vitro for at least 1 hour to about 24 hours and enriched for NK-CAR cells, further NK-CAR cells that are live and or demonstrate activated cell markers. The sorted and isolated cells may be stored in liquid nitrogen (−70° C.) for future use or directly infused in a subject in need thereof.

### Methods of Making CAR T Cells

T cells may be isolated from peripheral blood or leukapheresis samples, then genetically engineered ex vivo to generate CAR T cells. In some embodiments, a population of cells (for example, T cells, for example, T cells isolated from a frozen or fresh leukapheresis product) with an agent that stimulates a CD3/TCR complex and/or an agent that stimulates a costimulatory molecule on the surface of the cells.

In some embodiments, the population of T cells are electroporated, transfected or transduced with a vector or a nucleic acid molecule comprising a nucleic acid sequence encoding a CAR described herein. In some embodiment, a second, a third, a fourth or a fifth nucleic acid is also introduced into the T cells to activate the T cells, and/or to stabilize the T cells, and/or to protect the T cells from inhibitory signaling. In some embodiments, the T cells are further transfected or transduced with one, two, three, four, five, six, seven, eight, nine, ten or more nucleic acids, each expressing a different protein or fragment thereof in the T cell.

Provided herein are, inter alia, methods of making an T cell, comprising transducing, transfecting or electroporating the T cell with a vector described herein.

In some embodiments, the population of T cells are subjected to a transfection with a recombinant nucleic acid molecule comprising a nucleic acid sequence encoding a CAR in vitro.

In some embodiments, the population of T cells are subjected to a transduction with a recombinant nucleic acid molecule comprising a nucleic acid sequence encoding a CAR in vitro.

In some embodiments, transduction comprises transduction with a lentiviral vector, the vector comprising the nucleic acid molecule comprising a nucleic acid sequence encoding a CAR.

In some embodiments, the transduction comprises transduction with a lentiviral vector, the vector comprising the nucleic acid molecule comprising a nucleic acid sequence encoding a CAR.

In some embodiments, the transduction comprises transduction with an adenoviral vector, the vector comprising the nucleic acid molecule comprising a nucleic acid sequence encoding a CAR.

In some embodiments, the transduction comprises transduction with an adeno-associated viral (AAV) vector, the vector comprising the nucleic acid molecule comprising a nucleic acid sequence encoding a CAR.

In some embodiments, the population of T cells are transfected with a vector comprising the recombinant nucleic acid comprising a nucleic acid sequence encoding a CAR to obtain a population of CAR T cells, wherein the CAR T cells express the CAR. In some embodiments, the population of T cells are electroporated with a recombinant nucleic acid comprising a nucleic acid sequence encoding a CAR.

The recombinant nucleic acid molecule comprising a nucleic acid sequence encoding a CAR may be comprised in a vector. A vector may be a DNA vector. In addition to the nucleic acid sequence encoding a CAR the vector may comprise one or more additional sequences, such as a promoter, and enhancer, a 5′UTR, a 3′UTR, a poly A sequence, a localization sequence, a leader sequence or one or more further regulatory sequence. Exemplary promoter is a CMV promoter. An exemplary 3′ or 5′ UTR may be derived from a GCSF, GMCSF, beta actin, globulin or WPRE gene sequences, as is known to one of skill in the art.

In some embodiments, the recombinant nucleic acid molecule comprising a nucleic acid sequence encoding a CAR may comprise naked polynucleic acid. In some embodiments, the recombinant nucleic acid molecule comprising a nucleic acid sequence encoding a CAR is DNA. the recombinant nucleic acid molecule comprising a nucleic acid sequence encoding a CAR is RNA. the recombinant nucleic acid molecule comprising a nucleic acid sequence encoding a CAR is messenger RNA (mRNA).

Upon successful incorporation of the recombinant nucleic acid molecule comprising a nucleic acid sequence encoding a CAR in a population of T cells, at least 50%, at least 60%, at least 70%, at least 80%, at least 90%, at least 92%, at least 95%, or at least 100% cells of the population of T cells expresses the CAR. Accordingly, at least 50%, at least 60%, at least 70%, at least 80%, at least 90%, at least 92%, at least 95%, or at least 100% cells of the population of T cells may express CAR on their surface which can be determined in about 24 hours by a flow cytometric assay known to one of skill in the art. Accordingly, at least 50%, at least 60%, at least 70%, at least 80%, at least 90%, at least 92%, at least 95%, or at least 100% cells of the population of T cells that express CAR on their surface are CAR T cells. CAR T cells may be further selected and or enriched for the expression of CARs, for enhanced cytotoxicity profile, for enhanced cytokine secretion profile or a combination thereof.

In one aspect, provide herein is a T cell comprising a nucleic acid molecule disclosed herein or a vector disclosed herein.

In one aspect, provide herein is a pharmaceutical composition comprising a CAR molecule disclosed herein or an antibody molecule disclosed herein and a pharmaceutically acceptable carrier, excipient, or stabilizer. In some embodiments, the T cells are generated by ex vivo incorporation of vector or a nucleic acid molecule encoding the CAR described in any of the embodiments described herein, stabilizing the T cells in vitro comprising culturing the T cells comprising the nucleic acid encoding the CAR, in a medium comprising buffer, one or more growth factors, one or more cytokines, one or more chemokines one or more proteins, bioactive peptides or a combination thereof, for at least 1 hour, at least 3 hours, at least 6 hours, at least 10 hours, at least 12 hours, at least about 24 hours. In some embodiments, the CAR T cells are enriched from the population of T cells that are exposed, electroporated or otherwise transfected, or transduced with a nucleic acid molecule encoding the CAR. In some embodiments, the CAR T are enriched by sorting, for example in a flow cytometer. In some embodiments, the CAR T cells are enriched via positive selection sorting; in some embodiments, for example, alternatively the CAR T cells are selected by means of negative selection sorting, or a combination thereof. One of skill in the art is aware of positive and negative selection methods by FACS. In some embodiments, an aliquot of the CAR T cells are tested for functional efficacy, such as ability to bind to target cells for example by tetramer assay or by exposing the CAR T cells to cells expressing the target antigen on the cell surface, followed by imaging or other functional assays, ability to express activation markers and cytokines/chemokine production upon target binding, cytotoxic capability towards the target cells and so on. In some embodiments, a CAR T cell composition for adoptive transfer comprises at least 70%, at least 80%, at least 90% or at least 95% cells that are cytotoxic to a cell expressing the TRBC on the cell surface in vitro. In some embodiments, the population of CAR T cells comprises naïve T cells, as determined by, for example for example, CD45RA+CD45RO−CCR7+ staining. In some embodiments, the population of CAR T cells comprises about 5-40% naïve T cells, as determined by, for example for example, CD45RA+CD45RO−CCR7+ staining. In some embodiments, the CAR T cell population comprises about 1-14% central memory T cells, as determined by CD95+ staining. In some embodiments, the CAR T cell population comprises stem like memory T cells, that may be CD45RA+CD95+IL-2 b receptor+CCR7+CD62L+.

The population of T cells may be expanded for about 1 day, 2, days, 3 days, 4 days or 5 days, and prepared directly into a pharmaceutical composition for administering to a subject or storing for future use.

Following incorporation of a nucleic acid molecule comprising a nucleic acid sequence encoding a CAR in a population of T cells, and incubating the cells in a media for about 1 to about 24 hours in vitro, the cells may be stored in liquid nitrogen (−70° C.) for future use. Alternatively, in some embodiments, T cells may be stored in liquid nitrogen (−70° C.) for future use without delay following incorporation of said nucleic acid sequence encoding a CAR. In yet another embodiment, T cells may be sorted following incubation in a media in vitro for at least 1 hour to about 24 hours and enriched for CAR T cells, further CAR T cells that are live and or demonstrate activated cell markers. The sorted and isolated cells may be stored in liquid nitrogen (−70° C.) for future use or directly infused in a subject in need thereof.

### Pharmaceutical Compositions

CAR—expressing cells such as NK-CAR or CAR T cells generated as described above are formulated into a pharmaceutical composition for use for adoptive cell therapy. In some embodiments, cells may be frozen after preparation for use in future. Such cells are frozen in the buffer or excipient and can be directly administered to a subject in need thereof upon thawing the cells. Alternatively, cells are thawed, washed in a buffer, viability of the cells assessed, viable cells may be isolated, formulated into a pharmaceutical composition for delivery into a subject.

An excipient suitable for administration in vivo may comprise a sterile buffer, or an isotonic solution, such as saline, phosphate buffered saline, HEPES buffer.

In some embodiments an adequate dose (number) of NK-CAR cells in an NK-CAR cell pharmaceutical composition comprises at least 0.5×10\hat{ }6, 10\hat{ }6, 5×10\hat{ }6, 10\hat{ }7, 10\hat{ }8, 10\hat{ }9, 10\hat{ }10, 10\hat{ }11 or about 10\hat{ }12 NK-CAR cells, and a liquid medium or excipient, which is present at optimal pH and isotonicity for in vivo administration. In some embodiments, an administered dose of the NK-CAR cell composition comprises at least 0.5×10\hat{ }6, 10\hat{ }6, 5×10\hat{ }6, 10\hat{ }7, 10\hat{ }8, 10\hat{ }9, 10\hat{ }10, 10\hat{ }11 or about 10\hat{ }12 NK-CAR cells in a volume range of 1 ml to 20 ml. In some embodiments the composition comprises about 10\hat{ }6 NK-CAR cells-about 10\hat{ }11 NK-CAR cells in a suitable excipient for maintaining live cells and for suitability towards in vivo administration.

In some embodiments an adequate dose of CAR T cells in an CAR T cell pharmaceutical composition comprises at least 0.5×10\hat{ }6, 10\hat{ }6, 5×10\hat{ }6, 10\hat{ }7, 10\hat{ }8, 10\hat{ }9, 10\hat{ }10, 10\hat{ }11 or about 10 CAR T cells, and a liquid medium or excipient, which is present at optimal pH and isotonicity for in vivo administration. In some embodiments, an administered dose of the CAR T cell composition comprises at least 0.5×10\hat{ }6, 10\hat{ }6, 5×10\hat{ }6, 10\hat{ }7, 10\hat{ }8, 10\hat{ }9, 10\hat{ }10, 10\hat{ }11 or about 10\hat{ }12 CAR T cells in a volume range of 1 ml to 20 ml. In some embodiments the composition comprises about 10\hat{ }6 CAR T cells-about 10\hat{ }11 CAR T cells in a suitable excipient in vivo administration.

In some embodiments, the composition is administered subcutaneously. In some embodiments, the composition is administered intravenously.

### Methods of Treating Cancer

In one aspect, provided herein are methods of treating a cancer. In some embodiments, the methods comprise administering to a subject in need thereof a cell, such as a lymphocyte cell, expressing a CAR molecule disclosed herein or an antibody molecule disclosed herein, wherein the CAR molecule or antibody molecule is administered in an amount effective to treat the cancer. In some embodiments, the CAR molecule is expressed on the surface of a population of NK cells and said population of NK cells are administered to said subject in need thereof. In some embodiments, the NK cells are autologous to the subject being administered said NK cells. In some embodiments, the NK cells are allogenic to the subject being administered said NK cells.

In alternative embodiments, the CAR molecule is expressed on the surface of a population of T cells and said population of T cells are administered to said subject in need thereof. In some embodiments, the T cells are autologous to the subject being administered said T cells. In some embodiments, the T cells are allogenic to the subject being administered said T cells.

In some embodiments, provided herein are methods of treating a cancer in a subject. In some embodiments, the cancer is a lymphoma, a leukemia or a lymphocyte malignancy.

Accordingly, provided herein is a method of treating a T cell malignancy in a subject in need thereof, said method comprising administering to a subject in need thereof an NK cell composition, wherein said NK cell composition comprises a plurality of NK cells that express an exogenous nucleic acid molecule encoding a chimeric receptor (CAR) protein that comprises an anti-TRBC antigen binding domain (NK-CAR cells), wherein said CAR specifically binds to a TRBC protein. Accordingly, provided herein is a therapeutic composition, comprising a population of NK-CAR cells expressing a CAR comprising an anti-TRBC antigen binding domain for treating a T cell malignancy.

In some embodiments, provided herein is a method of treating a T cell malignancy in a subject in need thereof, said method comprising administering to a subject in need thereof a T cell composition, wherein said T cell composition comprises a plurality of T cells that express an exogenous nucleic acid molecule encoding a chimeric receptor (CAR) protein that comprises an anti-TRBC antigen binding domain, wherein said CAR specifically binds to a TRBC protein.

Accordingly, provided herein is a therapeutic composition, comprising a population of CAR T cells expressing a CAR comprising an anti-TRBC antigen binding domain for treating a T cell malignancy.

In some embodiments, the methods further comprise identifying, evaluating, or selecting a subject in need of treatment, wherein identifying, evaluating, or selecting comprises determining (e.g., directly determining or indirectly determining, e.g., obtaining information regarding) whether a subject has cancer cells that express a T cell receptor comprising TRBC1 or TRBC2. In some embodiments, the method further comprises: responsive to a determination that a subject has cancer cells that express a T cell receptor comprising TRBC1: optionally, selecting the subject for treatment with a CAR molecule comprising an antigen binding domain that binds to a T cell receptor comprising TRBC1 (or a population of NK cells or a population of T cells expressing said CAR molecule), and administering a CAR molecule (or a population of NK cells or a population of T cells expressing a CAR molecule) disclosed herein comprising an antigen binding domain that binds to a T cell receptor comprising TRBC1. In some embodiments, the method further comprises: responsive to a determination that a subject has cancer cells that express a T cell receptor comprising TRBC2: optionally, selecting the subject for treatment with a CAR molecule comprising an antigen binding domain that binds to a T cell receptor comprising TRBC2 (or a population of NK cells or a population of T cells expressing said CAR molecule), and administering a CAR molecule (or a population of NK cells or a population of T cells expressing a CAR molecule) disclosed herein comprising an antigen binding domain that binds to a T cell receptor comprising TRBC2.

In some embodiments, the therapeutic composition comprising NK cells for infusing (NK-CAR cell composition) comprises at least 60%, about 65%, about 70%, about 80%, about 90%, about 91%, about 92%, about 93%, about 94%, about 95%, about 96%, about 97%, about 98% about 99% or 100% of the NK cells expressing said chimeric receptor protein having the TRBC antigen binding domain.

In some embodiments, the therapeutic composition comprising T cells for infusing (CAR T cell composition) comprises at least 60%, about 65%, about 70%, about 80%, about 90%, about 91%, about 92%, about 93%, about 94%, about 95%, about 96%, about 97%, about 98% about 99% or 100% of the NK cells expressing said chimeric receptor protein having the TRBC antigen binding domain.

In one aspect, provided herein are methods of treating a cancer, e.g., a lymphoma or leukemia, e.g., a T cell lymphoma or leukemia, comprising: responsive to a determination that a subject has cancer cells that express a T cell receptor comprising TRBC1, administering to the subject a CAR molecule (or a population of NK cells or a population of T cells expressing a CAR molecule) disclosed herein, wherein the first antigen binding domain of the CAR molecule binds to TRBC1, wherein the CAR molecule (or a population of NK cells or a population of T cells expressing a CAR molecule) is administered in an amount effective to treat the cancer. In one aspect, provide herein is a method of treating a cancer, e.g., a lymphoma or leukemia, e.g., a T cell lymphoma or leukemia, comprising: responsive to a determination that a subject has cancer cells that express a T cell receptor comprising TRBC2, administering to the subject a CAR molecule (or a population of NK cells or a population of T cells expressing a CAR molecule) disclosed herein, wherein the first antigen binding domain of the CAR molecule (or a population of NK cells or a population of T cells expressing a CAR molecule) binds to TRBC2, wherein the CAR molecule is administered in an amount effective to treat the cancer.

In one aspect, provide herein is a method of identifying a subject in need of treatment for cancer, e.g., a lymphoma or leukemia, e.g., a T cell lymphoma or leukemia, using a CAR molecule disclosed herein, comprising determining (e.g., directly determining or indirectly determining, e.g., obtaining information regarding) whether a subject has cancer cells that express a T cell receptor comprising TRBC1 or TRBC2, wherein: responsive to a determination that the subject has cancer cells that express a T cell receptor comprising TRBC1, identifying the subject as a candidate for treatment using a CAR molecule (or a population of NK cells or a population of T cells expressing a CAR molecule) comprising an antigen binding domain that binds to TRBC1, and optionally not as a candidate for treatment using a CAR molecule comprising an antigen binding domain that binds to TRBC2, corresponsive to a determination that the subject has cancer cells that express a T cell receptor comprising TRBC2, identifying the subject as a candidate for treatment using a CAR molecule comprising an antigen binding domain that binds to TRBC2, and optionally not as a candidate for treatment using a CAR molecule comprising an antigen binding domain that binds to TRBC1.

In some embodiments, the method further comprises: responsive to identifying the subject as a candidate for treatment using a CAR molecule (or a population of NK cells or a population of T cells expressing a CAR molecule) comprising an antigen binding domain that binds to TRBC1, treating the subject with (e.g., administering to the subject) a CAR molecule (or a population of NK cells expressing a CAR molecule) comprising an antigen binding domain that binds to TRBC1, or responsive to identifying the subject as a candidate for treatment using a CAR molecule comprising an antigen binding domain that binds to TRBC2, treating the subject with (e.g., administering to the subject) a CAR molecule (or a population of NK cells or a population of T cells expressing a CAR molecule) comprising an antigen binding domain that binds to TRBC2.

In some embodiments of the aforementioned methods, the cancer is leukemia or lymphoma. In some embodiments, the cancer is Acquired immune deficiency syndrome (AIDS)-associated lymphoma, Angioimmunoblastic T-cell lymphoma, Adult T-cell leukemia/lymphoma, Burkitt lymphoma, Central nervous system (CNS) lymphoma, Diffuse large B-cell lymphoma (DLBCL), Lymphoblastic lymphoma, Mantle cell lymphoma (MCL), Peripheral T-cell lymphoma (PTCL) (e.g., Hepatosplenic T-cell lymphoma (HSGDTCL), Subcutaneous paniculitis-like T-cell lymphoma, or Enteropathy-associated T-cell lymphoma), Transformed follicular and transformed mucosa-associated lymphoid tissue (MALT) lymphomas, Cutaneous T-cell lymphoma (mycosis fungoides and Sezary syndrome), Follicular lymphoma, Lymphoplasmacytic lymphoma/Waldenstrom macroglobulinemia, Marginal zone B-cell lymphoma, Gastric mucosa-associated lymphoid tissue (MALT) lymphoma, Chronic lymphocytic leukemia/small-cell lymphocytic lymphoma (CLL/SLL), Extranodal T-/NK-cell lymphoma (nasal type), and Anaplastic large-cell lymphoma (e.g., primary cutaneous anaplastic large-cell lymphoma or systemic anaplastic large-cell lymphoma). In some embodiments, the cancer is lymphoma is Peripheral T-cell lymphoma (PTCL).

We claim:

1.-91. (canceled)

92. A composition comprising a chimeric antigen receptor (CAR) polypeptide, wherein the CAR comprises:
(i) an anti-TRBC antigen binding domain;
(ii) a transmembrane domain; and
(iii) an intracellular domain;
wherein the anti-TRBC antigen binding domain binds a TRBC1 protein or a TRBC2 protein; and
wherein:
(A) when the anti-TRBC antigen binding domain binds the TRBC1 protein, the anti-TRBC antigen binding domain comprises:
(a) a heavy chain variable domain (VH) comprising:
(1) a heavy chain complementarity determining region 1 (HCDR1) of SEQ ID NO: 9, a heavy chain complementarity determining region 2 (HCDR2) of SEQ ID NO: 11, and a heavy chain complementarity determining region 3 (HCDR3) of SEQ ID NO: 13; or
(2) a HCDR1 of SEQ ID NO: 44 or SEQ ID NO: 86, a HCDR2 of SEQ ID NO: 46 or SEQ ID NO: 95, and a HCDR3 of SEQ ID NO: 48; and
(b) a light chain variable domain (VL) comprising a light chain complementarity determining region 1 (LCDR1) of SEQ ID NO: 114 or SEQ ID NO: 149, a light chain complementarity determining region 2 (LCDR2) of SEQ ID NO: 116 or SEQ ID NO: 158, and a light chain complementarity determining region 3 (LCDR3) of SEQ ID NO: 118 or SEQ ID NO: 167;
wherein:
(1) the VH further comprises a VH framework region 1 (VHFWR1) of SEQ ID NO: 8 or SEQ ID NO: 22, a VH framework region 2 (VHFWR2) of SEQ ID NO: 10 or SEQ ID NO: 31, a VH framework region 4 (VHFWR4) of SEQ ID NO: 14 or SEQ ID NO: 77, or any combination thereof;
(2) the VL further comprises a VL framework region 1 (VLFWR1) of SEQ ID NO: 113, SEQ ID NO: 120, SEQ ID NO: 127, SEQ ID NO: 134, or SEQ ID NO: 141, a VL framework region 2 (VLFWR2) of SEQ ID NO: 115, SEQ ID NO: 122, or SEQ ID NO: 129, a VL framework region 3 (VLFWR3) of SEQ ID NO: 117 or SEQ ID NO: 124, a VL framework region 4 (VLFWR4) of SEQ ID NO: 119, or any combination thereof; or
(3) any combination thereof; or
(B) when the anti-TRBC antigen binding domain binds the TRBC2 protein, the anti-TRBC antigen binding domain comprises:
(a) a VH comprising a HCDR1 having the sequence of GX₁X₂MH, wherein X₁ is Y or F, and X₂ is P, H, V, Y, K, or A, a HCDR2 of SEQ ID NO: 306, and a HCDR3 of SEQ ID NO: 307; and
(b) a VL comprising a LCDR1 of SEQ ID NO: 309 or SEQ ID NO: 316, a LCDR2 of SEQ ID NO: 109, and a LCDR3 of SEQ ID NO: 118.

93. The composition of claim 92, wherein the anti-TRBC antigen binding domain binds the TRBC2 protein, and the anti-TRBC antigen binding domain comprises a VH comprising a HCDR1, a HCDR2, and a HCDR3 of
(i) SEQ ID NO: 222, SEQ ID NO: 11, and SEQ ID NO: 226, respectively,
(ii) SEQ ID NO: 37, SEQ ID NO: 11, and SEQ ID NO: 233, respectively,
(iii) SEQ ID NO: 37, SEQ ID NO: 11, and SEQ ID NO: 240, respectively,
(iv) SEQ ID NO: 243, SEQ ID NO: 11, and SEQ ID NO: 247, respectively,
(v) SEQ ID NO: 250, SEQ ID NO: 11, and SEQ ID NO: 247, respectively,
(vi) SEQ ID NO: 257, SEQ ID NO: 11, and SEQ ID NO: 247, respectively,
(vii) SEQ ID NO: 271, SEQ ID NO: 11, and SEQ ID NO: 247, respectively,
(viii) SEQ ID NO: 285, SEQ ID NO: 11, and SEQ ID NO: 247, respectively, or
(ix) SEQ ID NO: 292, SEQ ID NO: 11, and SEQ ID NO: 247, respectively.

94. The composition of claim 92, wherein the anti-TRBC antigen binding domain binds the TRBC2 protein, and the anti-TRBC antigen binding domain comprises:
(i) a VH further comprising a VHFWR2 of SEQ ID NO: 10, a VHFWR3 of SEQ ID NO: 12, a VHFWR4 of SEQ ID NO: 14, or any combination thereof;
(ii) a VL further comprising a VLFWR1 of SEQ ID NO: 113, a VLFWR2 of SEQ ID NO: 115, a VLFWR3 of SEQ ID NO: 117, a VLFWR4 of SEQ ID NO: 119, or any combination thereof; or
(iii) any combination thereof.

95. The composition of claim 92, wherein the anti-TRBC antigen binding domain binds the TRBC1 protein, and the anti-TRBC antigen binding domain comprises:
(i) a VH comprising a VHFWR1, a VHCDR1, a VHFWR2, a VHCDR2, a VHFWR3 a, VHCDR3, and a VHFWR4 of:
(1) SEQ ID NO: 8, SEQ ID NO: 9, SEQ ID NO: 10, SEQ ID NO: 11, SEQ ID NO: 12, SEQ ID NO: 13, and SEQ ID NO: 14, respectively;
(2) SEQ ID NO: 8, SEQ ID NO: 9, SEQ ID NO: 10, SEQ ID NO: 11, SEQ ID NO: 19, SEQ ID NO: 13, and SEQ ID NO: 14, respectively;
(3) SEQ ID NO: 22, SEQ ID NO: 9, SEQ ID NO: 10, SEQ ID NO: 11, SEQ ID NO: 26, SEQ ID NO: 13, and SEQ ID NO: 14, respectively;
(4) SEQ ID NO: 8, SEQ ID NO: 9, SEQ ID NO: 31, SEQ ID NO: 11, SEQ ID NO: 33, SEQ ID NO: 13, and SEQ ID NO: 14, respectively;
(5) SEQ ID NO: 8, SEQ ID NO: 44, SEQ ID NO: 10, SEQ ID NO: 11, SEQ ID NO: 12, SEQ ID NO: 13, and SEQ ID NO: 14, respectively;
(6) SEQ ID NO: 8, SEQ ID NO: 44, SEQ ID NO: 10, SEQ ID NO: 11, SEQ ID NO: 19, SEQ ID NO: 13, and SEQ ID NO: 14, respectively;
(7) SEQ ID NO: 22, SEQ ID NO: 44, SEQ ID NO: 10, SEQ ID NO: 11, SEQ ID NO: 26, SEQ ID NO: 13, and SEQ ID NO: 14, respectively;
(8) SEQ ID NO: 8, SEQ ID NO: 44, SEQ ID NO: 31, SEQ ID NO: 11, SEQ ID NO: 33, SEQ ID NO: 13, and SEQ ID NO: 14, respectively;
(9) SEQ ID NO: 22, SEQ ID NO: 44, SEQ ID NO: 10, SEQ ID NO: 11, SEQ ID NO: 26, SEQ ID NO: 13, and SEQ ID NO: 77, respectively;
(10) SEQ ID NO: 8, SEQ ID NO: 44, SEQ ID NO: 10, SEQ ID NO: 11, SEQ ID NO: 82, SEQ ID NO: 13, and SEQ ID NO: 77, respectively;
(11) SEQ ID NO: 22, SEQ ID NO: 86, SEQ ID NO: 10, SEQ ID NO: 11, SEQ ID NO: 26, SEQ ID NO: 13, and SEQ ID NO: 14, respectively;
(12) SEQ ID NO: 22, SEQ ID NO: 44, SEQ ID NO: 10, SEQ ID NO: 95, SEQ ID NO: 26, SEQ ID NO: 13, and SEQ ID NO: 14, respectively; or
(13) SEQ ID NO: 22, SEQ ID NO: 86, SEQ ID NO: 10, SEQ ID NO: 95, SEQ ID NO: 26, SEQ ID NO: 13, and SEQ ID NO: 14, respectively;
(ii) a VL comprising a VLFWR1, a VLCDR1, a VLFWR2, a VLCDR2, a VLFWR3 a, VLCDR3, and a VLFWR4 of:
(1) SEQ ID NO: 113, SEQ ID NO: 114, SEQ ID NO: 115, SEQ ID NO: 116, SEQ ID NO: 117, SEQ ID NO: 118, and SEQ ID NO: 119, respectively;
(2) SEQ ID NO: 120, SEQ ID NO: 114, SEQ ID NO: 122, SEQ ID NO: 116, SEQ ID NO: 124, SEQ ID NO: 118, and SEQ ID NO: 119, respectively;
(3) SEQ ID NO: 127, SEQ ID NO: 114, SEQ ID NO: 129, SEQ ID NO: 116, SEQ ID NO: 117, SEQ ID NO: 118, and SEQ ID NO: 119, respectively;
(4) SEQ ID NO: 134, SEQ ID NO: 114, SEQ ID NO: 115, SEQ ID NO: 116, SEQ ID NO: 117, SEQ ID NO: 118, and SEQ ID NO: 119, respectively;
(5) SEQ ID NO: 141, SEQ ID NO: 114, SEQ ID NO: 115, SEQ ID NO: 116, SEQ ID NO: 117, SEQ ID NO: 118, and SEQ ID NO: 119, respectively;
(6) SEQ ID NO: 127, SEQ ID NO: 149, SEQ ID NO: 129, SEQ ID NO: 116, SEQ ID NO: 117, SEQ ID NO: 118, and SEQ ID NO: 119, respectively;
(7) SEQ ID NO: 127, SEQ ID NO: 114, SEQ ID NO: 129, SEQ ID NO: 158, SEQ ID NO: 117, SEQ ID NO: 118, and SEQ ID NO: 119, respectively;
(8) SEQ ID NO: 127, SEQ ID NO: 114, SEQ ID NO: 129, SEQ ID NO: 116, SEQ ID NO: 117, SEQ ID NO: 167, and SEQ ID NO: 119, respectively; or
(9) SEQ ID NO: 127, SEQ ID NO: 149, SEQ ID NO: 129, SEQ ID NO: 158, SEQ ID NO: 117, SEQ ID NO: 167, and SEQ ID NO: 119, respectively; or
(iii) any combination thereof.

96. The composition of claim 92, wherein the anti-TRBC antigen binding domain binds the TRBC2 protein, and the anti-TRBC antigen binding domain comprises:
(i) a VH comprising a VHFWR1, a VHCDR1, a VHFWR2, a VHCDR2, a VHFWR3 a, VHCDR3, and a VHFWR4 of:
(1) SEQ ID NO: 221, SEQ ID NO: 222, SEQ ID NO: 10, SEQ ID NO: 11, SEQ ID NO: 12, SEQ ID NO: 226, and SEQ ID NO: 14, respectively;
(2) SEQ ID NO: 228, SEQ ID NO: 37, SEQ ID NO: 10, SEQ ID NO: 11, SEQ ID NO: 12, SEQ ID NO: 233, and SEQ ID NO: 14, respectively;
(3) SEQ ID NO: 235, SEQ ID NO: 37, SEQ ID NO: 10, SEQ ID NO: 11, SEQ ID NO: 12, SEQ ID NO: 240, and SEQ ID NO: 14, respectively;
(4) SEQ ID NO: 242, SEQ ID NO: 243, SEQ ID NO: 10, SEQ ID NO: 11, SEQ ID NO: 12, SEQ ID NO: 247, and SEQ ID NO: 14, respectively;
(5) SEQ ID NO: 249, SEQ ID NO: 250, SEQ ID NO: 10, SEQ ID NO: 11, SEQ ID NO: 12, SEQ ID NO: 247, and SEQ ID NO: 14, respectively;
(6) SEQ ID NO: 256, SEQ ID NO: 257, SEQ ID NO: 10, SEQ ID NO: 11, SEQ ID NO: 12, SEQ ID NO: 247, and SEQ ID NO: 14, respectively;
(7) SEQ ID NO: 263, SEQ ID NO: 250, SEQ ID NO: 10, SEQ ID NO: 11, SEQ ID NO: 12, SEQ ID NO: 247, and SEQ ID NO: 14, respectively;
(8) SEQ ID NO: 270, SEQ ID NO: 271, SEQ ID NO: 10, SEQ ID NO: 11, SEQ ID NO: 12, SEQ ID NO: 247, and SEQ ID NO: 14, respectively;
(9) SEQ ID NO: 277, SEQ ID NO: 243, SEQ ID NO: 10, SEQ ID NO: 11, SEQ ID NO: 12, SEQ ID NO: 247, and SEQ ID NO: 14, respectively;
(10) SEQ ID NO: 284, SEQ ID NO: 285, SEQ ID NO: 10, SEQ ID NO: 11, SEQ ID NO: 12, SEQ ID NO: 247, and SEQ ID NO: 14, respectively;
(11) SEQ ID NO: 291, SEQ ID NO: 292, SEQ ID NO: 10, SEQ ID NO: 11, SEQ ID NO: 12, SEQ ID NO: 247, and SEQ ID NO: 14, respectively; or
(12) SEQ ID NO: 298, SEQ ID NO: 243, SEQ ID NO: 10, SEQ ID NO: 11, SEQ ID NO: 12, SEQ ID NO: 247, and SEQ ID NO: 14, respectively;
(ii) a VL comprising a VLFWR1, a VLCDR1, a VLFWR2, a VLCDR2, a VLFWR3 a, VLCDR3, and a VLFWR4 of:
(1) SEQ ID NO: 113, SEQ ID NO: 309, SEQ ID NO: 115, SEQ ID NO: 109, SEQ ID NO: 117, SEQ ID NO: 118, and SEQ ID NO: 119, respectively; or
(2) SEQ ID NO: 113, SEQ ID NO: 316, SEQ ID NO: 115, SEQ ID NO: 109, SEQ ID NO: 117, SEQ ID NO: 118, and SEQ ID NO: 119, respectively; or
(iii) any combination thereof.

97. The composition of claim 92, wherein the anti-TRBC antigen binding domain binds the TRBC1 protein, and the anti-TRBC antigen binding domain comprises:
(i) a heavy chain comprising an amino acid sequence having at least 95% sequence identity to any one sequence selected from the group consisting of SEQ ID NOs: 176-185, 196, 197, 199, 200, 201, 203, 205, 207, 209, and 211;
(ii) a light chain comprising an amino acid sequence having at least 95% sequence identity to any one sequence selected from the group consisting of SEQ ID NOs: 186-195, 198, 202, 204, 206, 208, 210, and 212; or
(iii) any combination thereof.

98. The composition of claim 92, wherein the anti-TRBC antigen binding domain binds the TRBC1 protein, and the anti-TRBC antigen binding domain comprises:
(i) a heavy chain comprising an amino acid sequence having at least 95% sequence identity to SEQ ID NO: 176, and a light chain comprising an amino acid sequence having at least 95% sequence identity to SEQ ID NO: 186;
(ii) a heavy chain comprising an amino acid sequence having at least 95% sequence identity to any one sequence selected from the group consisting of SEQ ID NOs: 177, 178, 179, 180, 181, 182, 183, 184, and 185, and a light chain comprising an amino acid sequence having at least 95% sequence identity to any one sequence selected from the group consisting of SEQ ID NOs: 187, 188, 189, 190, 191, 192, 193, 194, and 195;
(iii) a heavy chain comprising an amino acid sequence having at least 95% sequence identity to any one sequence selected from the group consisting of SEQ ID NOs: 196, 197, and 199, and a light chain comprising an amino acid sequence having at least 95% sequence identity to SEQ ID NO: 198;
(iv) a heavy chain comprising an amino acid sequence having at least 95% sequence identity to any one sequence selected from the group consisting of SEQ ID NOs: 200 and 201, and a light chain comprising an amino acid sequence having at least 95% sequence identity to SEQ ID NO: 202;
(v) a heavy chain comprising an amino acid sequence having at least 95% sequence identity to SEQ ID NO: 203, and a light chain comprising an amino acid sequence having at least 95% sequence identity to SEQ ID NO: 204;
(vi) a heavy chain comprising an amino acid sequence having at least 95% sequence identity to SEQ ID NO: 205, and a light chain comprising an amino acid sequence having at least 95% sequence identity to SEQ ID NO: 206;
(vii) a heavy chain comprising an amino acid sequence having at least 95% sequence identity to SEQ ID NO: 207, and a light chain comprising an amino acid sequence having at least 95% sequence identity to SEQ ID NO: 208;
(viii) a heavy chain comprising an amino acid sequence having at least 95% sequence identity to SEQ ID NO: 209, and a light chain comprising an amino acid sequence having at least 95% sequence identity to SEQ ID NO: 210; or
(ix) a heavy chain comprising an amino acid sequence having at least 95% sequence identity to SEQ ID NO: 211, and a light chain comprising an amino acid sequence having at least 95% sequence identity to SEQ ID NO: 212.

99. The composition of claim 92, wherein the anti-TRBC antigen binding domain binds the TRBC1 protein, and the anti-TRBC antigen binding domain comprises:
(i) a heavy chain comprising an amino acid sequence selected from the group consisting of SEQ ID NOs: 177-185, 196, 197, 199, 200, 201, 203, 205, 207, 209, and 211;
(ii) a light chain comprising an amino acid sequence selected from the group consisting of SEQ ID NOs: 187-195, 198, 202, 204, 206, 208, 210, and 212; or
(iii) any combination thereof.

100. The composition of claim 92, wherein the anti-TRBC antigen binding domain binds the TRBC1 protein, and the anti-TRBC antigen binding domain comprises:
(i) a heavy chain comprising an amino acid sequence selected from the group consisting of SEQ ID NOs: 177, 178, 179, 180, 181, 182, 183, 184, and 185, and a light chain comprising an amino acid sequence selected from the group consisting of SEQ ID NOs: 187, 188, 189, 190, 191, 192, 193, 194, and 195;
(ii) a heavy chain comprising an amino acid sequence selected from the group consisting of SEQ ID NOs: 196, 197, and 199, and a light chain comprising the amino acid sequence of SEQ ID NO: 198;
(iii) a heavy chain comprising the amino acid sequence of SEQ ID NO: 200 or 201, and a light chain comprising the amino acid sequence of SEQ ID NO: 202;
(iv) a heavy chain comprising the amino acid sequence of SEQ ID NO: 203, and a light chain comprising the amino acid sequence of SEQ ID NO: 204;
(v) a heavy chain comprising the amino acid sequence of SEQ ID NO: 205, and a light chain comprising the amino acid sequence of SEQ ID NO: 206;
(vi) a heavy chain comprising the amino acid sequence of SEQ ID NO: 207, and a light chain comprising the amino acid sequence of SEQ ID NO: 208;
(vii) a heavy chain comprising the amino acid sequence of SEQ ID NO: 209, and a light chain comprising the amino acid sequence of SEQ ID NO: 210; or
(viii) a heavy chain comprising the amino acid sequence of SEQ ID NO: 211, and a light chain comprising the amino acid sequence of SEQ ID NO: 212.

101. The composition of claim 92, wherein the anti-TRBC antigen binding domain binds the TRBC2 protein, and the anti-TRBC antigen binding domain comprises:
(i) a heavy chain comprising an amino acid sequence having at least 95% sequence identity to any one sequence selected from the group consisting of SEQ ID NOs: 325-336;
(ii) a light chain comprising an amino acid sequence having at least 95% sequence identity to SEQ ID NO: 337 or SEQ ID NO: 338; or
(iii) any combination thereof.

102. The composition of claim 92, wherein the anti-TRBC antigen binding domain binds the TRBC2 protein, and the anti-TRBC antigen binding domain comprises:
(i) a heavy chain comprising an amino acid sequence selected from the group consisting of SEQ ID NOs: 325-336;
(ii) a light chain comprising the amino acid sequence of SEQ ID NO: 337 or SEQ ID NO: 338; or
(iii) any combination thereof.

103. The composition of claim 92, wherein the anti-TRBC antigen binding domain binds the TRBC2 protein, and the anti-TRBC antigen binding domain comprises an amino acid sequence having at least 95% sequence identity to any one sequence selected from the group consisting of SEQ ID NOs: 339-343.

104. The composition of claim 92, wherein the anti-TRBC antigen binding domain binds the TRBC2 protein, and the anti-TRBC antigen binding domain comprises an amino acid sequence selected from the group consisting of SEQ ID NOs: 339-343.

105. The composition of claim 92, wherein:
(i) the transmembrane domain comprises a CD8a transmembrane domain or a CD28 transmembrane domain;
(ii) the intracellular domain comprises an intracellular signaling domain selected from the group consisting of a CD3ζ intracellular signaling domain, a CD137 intracellular signaling domain, a CD28 intracellular signaling domain, and a CD8 intracellular signaling domain; or
(iii) a combination thereof.

106. The composition of claim 92, wherein the transmembrane domain further comprises a CD8a hinge region.

107. The composition of claim 92, wherein the composition comprises a population of cells that express the CAR polypeptide.

108. The composition of claim 107, wherein the population of cells are a population of T cells or a population of NK cells.

109. A pharmaceutical composition comprising the composition of claim 107, and a pharmaceutically acceptable excipient.

110. A method of treating a T cell malignancy in a subject in need thereof comprising:
administering to the subject a therapeutically effective amount of the pharmaceutical composition of claim 109, wherein the population of cells kills a cancer cell expressing the TRBC protein when administered to the subject, thereby treating the T cell malignancy in the subject.

111. The method of claim 110, wherein the population of cells express TNF-alpha, interferon gamma, IL-1b, IL-6, IL-7, IL-10, IL-12p40, IFN-alpha, MIP-1alpha, MIP-1beta, RANTES, MIG (CXCL9), CD56, ICAM-1, CD27, CD48, CD107a, or any combination thereof upon contacting a cell expressing the TRBC protein on the cell surface in vitro or in vivo.

112. The method of claim 110, wherein the population of cells are allogenic or autologous to the subject.

113. The method of claim 110, wherein the T cell malignancy is selected from the group consisting of a T cell lymphoma, a non-Hodgkin's lymphoma, an angioimmunoblastic T cell lymphoma, an anaplastic large cell lymphoma, and an acute lymphoblastic leukemia.
